(19) United States
(12) Patent Application Publication
(10) Pub. No.: US 2021/0154190 A1
(43) Pub. Date: May 27, 2021

(54) SHP2 INHIBITOR COMPOSITIONS AND METHODS FOR TREATING CANCER

(71) Applicant: Revolution Medicines, Inc., Redwood City, CA (US)

(72) Inventors: David E. WILDES, Redwood City, CA (US); Carlos STAHLHUT-ESPINOSA, Redwood City, CA (US); Robert J. NICHOLS, Redwood City, CA (US)

(21) Appl. No.: 17/064,317

(22) Filed: Oct. 6, 2020

Related U.S. Application Data
(63) Continuation of application No. PCT/US2019/026543, filed on Apr. 9, 2019.
(60) Provisional application No. 62/655,648, filed on Apr. 10, 2018.

(51) Int. Cl. A61K 31/497; A61K 31/551; A61K 31/4965; A61K 31/55; A61K 31/517; A61P 35/00

(52) U.S. Cl. CPC A61K 31/497; A61K 31/551; A61P 35/00; A61K 31/55; A61K 31/517; A61K 31/4965

(57) ABSTRACT

The present disclosure provides methods of treating diseases or disorders related to mutations in the SHP2 gene using allosteric inhibitors of SHP2 and methods and diagnostic tests for identifying subjects susceptible or resistant to allosteric inhibitors of SHP2. In particular, the present disclosure provides allosteric inhibitor-sensitive mutations and allosteric inhibitor-resistant mutations of SHP2 for diagnostic and therapeutic use.

### CROSS REFERENCE TO RELATED APPLICATIONS

This application is a continuation of International Patent Application No. PCT/US2019/026543, filed Apr. 9, 2019, which claims the benefit of U.S. Provisional Application No. 62/655,648, filed Apr. 10, 2018, the contents of each of which is incorporated herein by reference in their entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is REME_010_01WO_ST25.txt. The text file is 5.75 KB, was created on Mar. 27, 2019, and is being submitted electronically via EFS-Web.

### FIELD OF THE INVENTION

The present disclosure relates to methods for the treatment of diseases or disorders (e.g., cancer or an inherited developmental disorder) with inhibitors of the protein tyrosine phosphatase SHP2. Specifically, this invention is concerned with methods of treating diseases or disorders (such as cancer or inherited developmental disorder) in subjects that are identified as candidates for treatment with an allosteric SHP2 inhibitor.

### BACKGROUND OF THE INVENTION

SHP2 is a non-receptor protein tyrosine phosphatase encoded by the PTPN11 gene that contributes to multiple cellular functions including proliferation, differentiation, cell cycle maintenance and migration. SHP2 is involved in signaling through the RAS-mitogen-activated protein kinase (MAPK), the JAK-STAT and/or the phosphoinositol 3-kinase-AKT pathways.

SHP2 has two N-terminal Src homology 2 domains (N—SH2 and C—SH2), a catalytic domain (PTP), and a C-terminal tail. The two SH2 domains control the subcellular localization and functional regulation of SHP2. The molecule exists in an inactive, self-inhibited conformation stabilized by a binding network involving residues from both the N—SH2 and PTP domains. Stimulation by, for example, cytokines or growth factors acting through RTKs leads to exposure of the catalytic site resulting in enzymatic activation of SHP2.

Mutations in the PTPN11 gene and subsequently in SHP2 have been identified in several human developmental diseases, such as Noonan Syndrome and LEOPARD Syndrome, as well as human cancers, such as juvenile myelomonocytic leukemias, neuroblastoma, melanoma, acute myeloid leukemia and cancers of the breast, lung and colon. Some of these mutations destabilize the autoinhibited conformation of SHP2 and promote autoactivation or enhanced growth factor-driven activation of SHP2.

SHP2, therefore, represents a highly attractive target for the development of novel therapies for the treatment of various diseases including cancer. Either the knockdown of SHP2 expression using RNAi technology or inhibition of SHP2 by an allosteric small molecule inhibitor interferes with signaling from various RTKs involved in driving cancer cell growth. (Chen, Ying-Nan P. 148 Nature Vol 535 7 Jul. 2016 at pg. 151).

It has been disclosed previously, however, that allosteric SHP2 inhibitors show reduced potency against clinically-relevant SHP2 mutants when the mutant SHP2 is in an activated state. Thus, there exists an unmet need for methods for treating a disease or disorder associated with cells containing a mutant SHP2, and for methods for identifying a subject as susceptible or resistant to a SHP2 inhibitor, as well as diagnostic tests for the same.

### SUMMARY OF THE INVENTION

The present disclosure relates to methods of treating diseases or disorders (such as cancer or inherited developmental disorder) in certain subsets of subjects that are determined to be candidates for treatment with an allosteric SHP2 inhibitor.

In one aspect, the disclosure provides a method of treating a subject having a disease or disorder associated with cells containing a mutant SHP2, comprising administering to the subject an allosteric SHP2 inhibitor, wherein the mutant SHP2 comprises an allosteric inhibitor-sensitive mutation. In embodiments of the method, the allosteric inhibitor-sensitive mutation is F285S, L262R, S189A, D61G, E69K, T73I, or Q506P. In embodiments of the method, the cells are negative for an allosteric inhibitor-resistant mutation of SHP2. In embodiments of the method, the allosteric inhibitor-resistant mutation is E76K, P491S, or S502P.

In one aspect, the disclosure provides a method of identifying a subject with SHP2 mutations susceptible to a SHP2 inhibitor, comprising genotyping a biological sample from the subject for SHP2 mutations, wherein the subject is identified as susceptible to the SHP2 inhibitor if the SHP2 mutations comprise an allosteric inhibitor-sensitive mutation. In embodiments of the method, the allosteric inhibitor-sensitive mutation is F285S, L262R, S189A, D61G, E69K, T73I, or Q506P.

In one aspect, the disclosure provides a method of identifying a subject as resistant to an allosteric SHP2 inhibitor, comprising genotyping a biological sample from the subject for SHP2 mutations, wherein the subject is identified as resistant to the SHP2 inhibitor if the SHP2 mutations comprise an allosteric inhibitor-resistant mutation. In embodiments of the method, the allosteric inhibitor-resistant mutation is E76K, P491S, or S502P.

In one aspect, the disclosure provides a diagnostic test for allosteric SHP2 inhibitor sensitivity, comprising a nucleic acid probe specific for an allosteric inhibitor-sensitive mutation of SHP2. In embodiments of the diagnostic method, the allosteric inhibitor-sensitive mutation is F285S, L262R, S189A, D61G, E69K, T73I, or Q506P.

### BRIEF DESCRIPTION OF THE DRAWINGS

FIG. 1 shows a simple equilibrium model for activation/inhibition by peptide binding, mutation, and inhibitor binding.

FIG. 2 shows the potency of each compound to inhibit non-activated mutant SHP2 plotted versus the potency to inhibit wild-type SHP2.

FIG. 3 shows the potency of each compound to inhibit peptide-activated mutant SHP2 plotted versus the potency to inhibit peptide-activated wild-type SHP2.

FIG. 4 shows negligible shift in potency for inhibition of wild-type SHP2 between non-activated and peptide-activated biochemical experiments.

FIG. 5 shows addition of activating peptide (NsCs, 0.5 μM) had negligible effect on inhibitor potency for WT SHP2 and varying effects on mutants S189A (FIG. 5A), F285C (FIG. 5B), D61G (FIG. 5C), and E76K (FIG. 5D).

FIG. 6 shows the generation of isogenic cell lines for SHP2 mutants and their use in cellular assays for SHP2 inhibition.

FIG. 7 shows EGF-induced pERK activity for various mutant SHP2s at various concentrations of Compound B

FIG. 8 shows that biochemical data from activated SHP2 is a better predictor of cellular sensitivity than biochemical data from unactivated SHP2. FIG. 8A depicts biochemical pIC₅₀ plotted against cellular pIC₅₀ for activated SHP2. FIG. 8B depicts biochemical pIC₅₀ plotted against cellular pIC₅₀ for unactivated SHP2.

### DETAILED DESCRIPTION OF THE INVENTION

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### General Methods

The practice of the present invention will employ, unless otherwise indicated, conventional techniques of cell culturing, molecular biology (including recombinant techniques), microbiology, cell biology, biochemistry and immunology, which are within the skill of the art. Such techniques are explained fully in the literature, such as, Molecular Cloning: A Laboratory Manual, third edition (Sambrook et al., 2001) Cold Spring Harbor Press; Oligonucleotide Synthesis (P. Herdewijn, ed., 2004); Animal Cell Culture (R. I. Freshney), ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Manual of Clinical Laboratory Immunology (B. Detrick, N. R. Rose, and J. D. Folds eds., 2006); Immunochemical Protocols (J. Pound, ed., 2003); Lab Manual in Biochemistry: Immunology and Biotechnology (A. Nigam and A. Ayyagari, eds. 2007); Immunology Methods Manual: The Comprehensive Sourcebook of Techniques (Ivan Lefkovits, ed., 1996); Using Antibodies: A Laboratory Manual (E. Harlow and D. Lane, eds., 1988); and others.

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used in this disclosure to refer to one or more than one (i.e., to at least one) of the grammatical object of the article. By way of example, “an element” means one element or more than one element.

The term “and/of” is used in this disclosure to mean either “and” or “or” unless indicated otherwise.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “e.g.” is used herein to mean “for example,” and will be understood to imply the inclusion of a stated step or element or group of steps or elements but not the exclusion of any other step or element or group of steps or elements.

By “optional” or “optionally,” it is meant that the subsequently described event or circumstance may or may not occur, and that the description includes instances where the event or circumstance occurs and instances in which it does not. For example, “optionally substituted aryl” encompasses both “aryl” and “substituted aryl” as defined herein. It will be understood by those ordinarily skilled in the art, with respect to any group containing one or more substituents, that such groups are not intended to introduce any substitution or substitution patterns that are sterically impractical, synthetically non-feasible, and/or inherently unstable.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The terms “Compound A”, “Cmp A”, “Compound 1” and “Cmp 1” are used interchangeably herein to refer to RMC-0693943 (abbreviated herein as “RMC-3943”), which has the following structure:

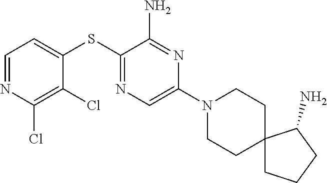 [SMILES: Nc1nc(N2CCC3(CCC[C@H]3N)CC2)cnc1Sc1ccnc(Cl)c1Cl]

The terms “Compound B”, “Cmp B”, “Compound 21” and “Cmp 21” are used interchangeably herein to refer to RMC-0694550 (abbreviated herein as “RMC-4550”), which has the following structure:

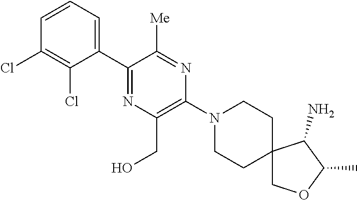 [SMILES: Cc1nc(N2CCC3(CC2)CO[C@@H](C)[C@H]3N)c(CO)nc1-c1cccc(Cl)c1Cl]

The term “Compound C” and “Cmp C” are used interchangeably herein to refer to an allosteric SHP2 inhibitor compound of similar structure to Compounds A and B. Compound C is disclosed in PCT/US2017/041577 (WO 2018/013597), incorporated herein by reference in its entirety.

The term SHP099 refers to a SHP2 inhibitor having the following structure:

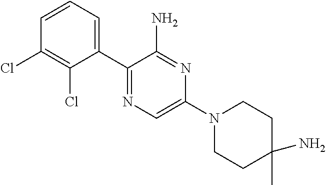 [SMILES: CC1(N)CCN(c2cnc(-c3cccc(Cl)c3Cl)c(N)n2)CC1]

The term “disorder” is used in this disclosure to mean, and is used interchangeably with, the terms disease, condition, or illness, unless otherwise indicated.

An “effective amount” when used in connection with a compound is an amount effective for treating or preventing a disease or disorder in a subject as described herein.

The term “inhibitor” means a compound that prevents a biomolecule, (e.g., a protein, nucleic acid) from completing or initiating a reaction. An inhibitor can inhibit a reaction by competitive, uncompetitive, or non-competitive means. Exemplary inhibitors include, but are not limited to, nucleic acids, DNA, RNA, shRNA, siRNA, proteins, protein mimetics, peptides, peptidomimetics, antibodies, small molecules, chemicals, analogs that mimic the binding site of an enzyme, receptor, or other protein, e.g., that is involved in signal transduction, therapeutic agents, pharmaceutical compositions, drugs, and combinations of these. In some embodiments, the inhibitor can be nucleic acid molecules including, but not limited to, siRNA that reduce the amount of functional protein in a cell. Accordingly, compounds said to be “capable of inhibiting” a particular protein, e.g., SHP2, comprise any such inhibitor.

The term “allosteric inhibitor” means a small-molecule compound capable of inhibiting SHP2 through binding to SHP2 at a site other than the active site of the enzyme. Exemplary allosteric SHP2 inhibitors disclosed herein include, without limitation: (i) Compound A; (ii) Compound B; (iii) Compound C; (iv) SHP099; (v) an allosteric SHP2 inhibitor compound of any one of Formula I, of Formula II, of Formula III, of Formula I-V1, of Formula I-V2, of Formula I-W, of Formula I-X, of Formula I-Y, of Formula I-Z, of Formula IV, of Formula V, of Formula VI, of Formula IV-X, of Formula IV-Y, of Formula IV-Z, of Formula VII, of Formula VIII, of Formula IX, and of Formula X; (vi) TNO155; (vii) a SHP2 inhibitor disclosed in international PCT application PCT/US2017/041577 (WO2018013597), incorporated herein by reference in its entirety; (viii) a compound from Table A1, disclosed herein; (ix) a compound from Table A2, disclosed herein; and (x) a combination thereof.

The term “modulating” includes “increasing,” “enhancing” or “stimulating,” as well as “decreasing” or “reducing,” typically in a statistically significant or a physiologically significant amount as compared to a control. An “increased,” “stimulated” or “enhanced” amount is typically a “statistically significant” amount, and may include an increase that is 1.1, 1.2, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the amount produced by no composition (e.g., in the absence of an agent or compound) or a control composition, sample or test subject. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease in the amount produced by no composition (the absence of an agent or compound) or a control composition, including all integers in between.

The term “mutation” as used herein indicates any modification of a nucleic acid and/or polypeptide which results in an altered nucleic acid or polypeptide. The term “mutation” may include, for example, point mutations, deletions or insertions of single or multiple residues in a polynucleotide, which includes alterations arising within a protein-encoding region of a gene as well as alterations in regions outside of a protein-encoding sequence, such as, but not limited to, regulatory or promoter sequences, as well as amplifications and/or chromosomal breaks or translocations.

The term “allosteric inhibitor-sensitive mutation,” when used in reference to a SHP2 mutation, means a mutation in SHP2 that results in a SHP2 polypeptide that may be modulated by a SHP2 allosteric inhibitor (e.g., any one of the SHP2 allosteric inhibitors disclosed herein). As will be clear to one of skill in the art, such modulation of a SHP2 polypeptide comprising an allosteric inhibitor-sensitive mutation will in some embodiments result in a decrease in the activity of the SHP2 polypeptide. Such activity may be measured using any suitable activity assay known in the art or disclosed herein (see, e.g., the SHP2 allosteric inhibition assay described herein in Example 1). In some embodiments, the allosteric inhibitor-sensitive mutation is a SHP2 mutation selected from any one of F285S, L262R, S189A, D61G, E69K, T73I, and Q506P. In some embodiments, the allosteric inhibitor-sensitive mutation may be a combination of two or more SHP2 mutations selected from F285S, L262R, S189A, D61G, E69K, T73I, and Q506P.

The term “allosteric inhibitor-resistant mutation” when used in reference to a SHP2 mutation, means a mutation in SHP2 that renders a SHP2 polypeptide refractory or resistant to inhibition with a SHP2 allosteric inhibitor. Thus, in some embodiments, an allosteric inhibitor-resistant mutation in a SHP2 polypeptide decreases the inhibitory effect that a SHP2 allosteric inhibitor has on the SHP2 polypeptide as compared to the effect the inhibitor has on a similar SHP2 polypeptide differing only in the absence of the allosteric inhibitor-resistant mutation. Such activity may be measured using any suitable activity assay known in the art or disclosed herein (see, e.g., the SHP2 allosteric inhibition assay described herein in Example 1). In some embodiments, an allosteric inhibitor-resistant mutation in a SHP2 polypeptide abolishes all detectable inhibitory effects that a SHP2 allosteric inhibitor has on the activity of the SHP2 polypeptide, wherein the inhibitor has detectable inhibitory efficacy on a similar SHP2 polypeptide differing only in the absence of the allosteric inhibitor-resistant mutation. Such allosteric inhibitor-resistant mutations include, without limitation, mutations that destabilize the autoinhibited conformation of SHP2. In some embodiments, the allosteric inhibitor-resistant mutation is a SHP2 mutation selected from any one of E76K, P491S, and S502P. In some embodiments, the allosteric inhibitor-resistant mutation is a combination of two or more SHP2 mutations selected from E76K, P491S, and S502P.

A “patient” or “subject” is a mammal, e.g., a human, mouse, rat, guinea pig, dog, cat, horse, cow, pig, or non-human primate, such as a monkey, chimpanzee, baboon or rhesus.

The term “prevent” or “preventing” with regard to a subject refers to keeping a disease or disorder from afflicting the subject. Preventing includes prophylactic treatment. For instance, preventing can include administering to the subject a compound disclosed herein before a subject is afflicted with a disease and the administration will keep the subject from being afflicted with the disease.

The term “providing to a/the subject” a therapeutic agent, e.g., a SHP2 inhibitor, includes administering such an agent.

The terms “RAS pathway” and “RAS/MAPK pathway” are used interchangeably herein to refer to a signal transduction cascade downstream of various cell surface growth factor receptors in which activation of RAS (and its various isoforms and alleotypes) is a central event that drives a variety of cellular effector events that determine the proliferation, activation, differentiation, mobilization, and other functional properties of the cell. SHP2 conveys positive signals from growth factor receptors to the RAS activation/deactivation cycle, which is modulated by guanine nucleotide exchange factors (GEFs, such as SOS1) that load GTP onto RAS to produce functionally active GTP-bound RAS as well as GTP-accelerating proteins (GAPs, such as NF1) that facilitate termination of the signals by conversion of GTP to GDP. GTP-bound RAS produced by this cycle conveys essential positive signals to a series of serine/threonine kinases including RAF and MAP kinases, from which emanate additional signals to various cellular effector functions.

The terms “RAS pathway mutation” and “RAS/MAPK pathway activating mutation” are used interchangeably herein to refer to a mutation in a gene encoding a protein directly involved in the signaling processes of the RAS/MAPK signaling pathway and/or regulating (either positively or negatively) this signaling pathway that renders the pathway active, wherein such mutation may increase, change or decrease the activity level of said protein. Such proteins include but are not limited to Ras, Raf, NF1, SOS, and specific isoforms or alleotypes thereof.

The term “RTK-driven tumor” refers to a tumor comprising a cell with one or more oncogenic mutation of an RTK, or a protein that is part of the RTK signaling complex, that causes high levels RTK signaling. Some such cells may be considered “addicted” to the RTK, and inhibition of RTK signaling leads to simultaneous suppression of downstream pathways, often resulting in cell growth, arrest, and death. RTK-driven tumors include, but are not limited to, non-small cell lung cancers (NSCLCs) with mutations in EGFR or ALK.

The term “SHP2” means “Src Homology 2 domain-containing protein tyrosine phosphatase 2” and is also known as SH-PTP2, SH-PTP3, Syp, PTP1D, PTP2C, SAP-2 or PTPN11. Numbering of SHP2 mutations in the present disclosure is according to Uniprot Isoform 2 (accession number Q06124-2) (SEQ ID NO: 1):

```
              10 20 30 40
MTSRRWFHPN ITGVEAENLL LTRGVDGSFL ARPSKSNPGD
              50 60 70 80
FTLSVRRNGA VTHIKIQNTG DYYDLYGGEK FATLAELVQY
                90 100 110 120
YMEHHGQLKE KNGDVIELKY PLNCADPTSE RWFHGHLSGK
              130 140 150 160
EAEKLLTEKG KHGSFLVRES QSHPGDFVLS VRTGDDKGES
              170 180 190 200
NDGKSKVTHV MIRCQELKYD VGGGERFDSL TDLVEHYKKN
              210 220 230 240
PMVETLGTVL QLKQPLNTTR INAAEIESRV RELSKLAETT
              250 260 270 280
DKVKQGFWEE FETLQQQECK LLYSRKEGQR QENKNKNRYK
              290 300 310 320
NILPFDHTRV VLHDGDPNEP VSDYINANII MPEFETKCNN
              330 340 350 360
SKPKKSYIAT QGCLQNTVND FWRMVFQENS RVIVMTTKEV
              370 380 390 400
ERGKSKCVKY WPDEYALKEY GVMRVRNVKE SAAHDYTLRE
              410 420 430 440
LKLSKVGQGN TERTVWQYHF RTWPDHGVPS DPGGVLDFLE
              450 460 470 480
EVHHKQESIM DAGPVVVHCS AGIGRTGTFI VIDILIDIIR
              490 500 510 520
EKGVDCDIDV PKTIQMVRSQ RSGMVQTEAQ YRFIYMAVQH
              530 540 550 560
YIETLQRRIE EEQKSKRKGH EYTNIKYSLA DQTSGDQSPL
              570 580 590
PPCTPTPPCA EMREDSARVY ENVGLMQQQK SFR
```

The convention “AAwt ###AAmut” is used to indicate a mutation that results in the wild-type amino acid AAwt at position ### in the polypeptide being replaced with mutant AAmut.

A “therapeutic agent” is any substance, e.g., a compound or composition, capable of treating a disease or disorder. In some embodiments, therapeutic agents that are useful in connection with the present disclosure include without limitation SHP2 inhibitors, ALK inhibitors, MEK inhibitors, RTK inhibitors (TKIs), and cancer chemotherapeutics. Many such inhibitors are known in the art and are disclosed herein.

The terms “therapeutically effective amount”, “therapeutic dose”, “prophylactically effective amount”, or “diagnostically effective amount” is the amount of the drug, e.g., a SHP2 inhibitor, needed to elicit the desired biological response following administration.

The term “treatment” or “treating” with regard to a subject, refers to improving at least one symptom, pathology or marker of the subject's disease or disorder, either directly or by enhancing the effect of another treatment. Treating includes curing, improving, or at least partially ameliorating the disorder, and may include even minimal changes or improvements in one or more measurable markers of the disease or condition being treated. “Treatment” or “treating” does not necessarily indicate complete eradication or cure of the disease or condition, or associated symptoms thereof. The subject receiving this treatment is any subject in need thereof. Exemplary markers of clinical improvement will be apparent to persons skilled in the art.

### Overview

The present disclosure relates to, inter alia, compositions, methods, and kits for treating or preventing a disease or disorder (e.g., cancer) with a SHP2 inhibitor alone or in combination with another suitable therapeutic agent.

SHP2 is an important signaling effector molecule for a variety of receptor tyrosine kinases (RTKs), including the receptors of platelet-derived growth factor (PDGFR), fibroblast growth factor (FGFR), and epidermal growth factor (EGFR). SHP2 is also an important signaling molecule that regulates the activation of the mitogen activated protein (MAP) kinase pathway which can lead to cell transformation, a prerequisite for the development of cancer. For example, SHP2 is involved in signaling through the Ras-mitogen-activated protein kinase, the JAK-STAT and/or the phosphoinositol 3-kinase-AKT pathways. SHP2 mediates activation of Erk1 and Erk2 (Erk1/2, Erk) MAP kinases by receptor tyrosine kinases such as ErbB1, ErbB2 and c-Met by modulating RAS activation.

SHP2 has two N-terminal Src homology 2 domains (N—SH2 and C—SH2), a catalytic domain (PTP), and a C-terminal tail. The two SH2 domains control the subcellular localization and functional regulation of SHP2. The molecule exists in an inactive conformation, inhibiting its own activity via a binding network involving residues from both the N—SH2 and PTP domains. In response to growth factor stimulation, SHP2 associates with the RTK signaling apparatus, and this induces a conformational change that results in SHP2 activation.

Activating mutations of SHP2 have been associated with developmental pathologies such as Noonan syndrome and LEOPARD Syndrome and may also be found in multiple cancer types, including most RTK-driven tumors, leukemia, lung and breast cancer, gastric carcinoma, anaplastic large-cell lymphoma, glioblastoma and neuroblastoma. (Grossmann, K. S., Rosario, M., Birchmeier, C. & Birchmeier, W. The tyrosine phosphatase Shp2 in development and cancer. Adv. Cancer Res. 106, 53-89 (2010). Chan, R. J. & Feng, G. S. PTPN11 is the first identified proto-oncogene that encodes a tyrosine phosphatase. Blood 109, 862-867 (2007). Matozaki, T., Murata, Y., Saito, Y., Okazawa, H. & Ohnishi, H. Protein tyrosine phosphatase SHP-2: a proto-oncogene product that promotes Ras activation. Cancer Sci. 100, 1786-1793 (2009). Mohi, M. G. & Neel, B. G. The role of Shp2 (PTPN11) in cancer. Curr. Opin. Genet. Dev. 17, 23-30 (2007).) Östman, A., Hellberg, C. & Böhmer, F. D. Protein-tyrosine phosphatases and cancer. Nat. Rev. Cancer 6, 307-320 (2006).

In addition, SHP2 plays a role in transducing signals originating from immune checkpoint molecules, including but not limited to programmed cell death protein 1 (PD-1) and cytotoxic T-lymphocyte-associated protein 4 (CTLA-4). In this context, inhibition of SHP2 function may promote activation of immune cells expressing checkpoint molecules, including anti-cancer immune responses.

It has been disclosed previously that either the knockdown of SHP2 expression using RNAi technology or inhibition of SHP2 by an allosteric small molecule inhibitor interferes with signaling from various RTKs involved in driving cancer cell growth. (Chen, Ying-Nan P. 148 Nature Vol 535 7 Jul. 2016 at pg. 151).

In some embodiments, the present disclosure provides a method for patient stratification based upon the presence or absence of a SHP2 mutation or based upon the particular subtype of such a mutation. As used herein, “patient stratification” means classifying one or more patient as having a disease or disorder (e.g., cancer) that is either likely or unlikely to be treatable with an allosteric SHP2 inhibitor. Patient stratification may comprise classifying a patient as having a tumor that is sensitive to treatment with an allosteric SHP2 inhibitor. The patient stratification may be based on the presence or absence of a tumor comprising one or more cell containing a SHP2 mutation that renders the mutated SHP2 protein sensitive or resistant to allosteric inhibitors of SHP2.

Any disease or condition associated with a SHP2 mutation may be identified, assessed, and/or treated according to the present disclosure. In particular embodiments, the SHP2 mutation leaves the mutated protein sensitive to allosteric inhibitors of SHP2. Several such diseases or conditions comprising SHP2 mutations are known in the art. For example, in certain embodiments, the present disclosure provides methods for treating a disease or condition selected from, but not limited to, Noonan Syndrome (e.g., Noonan syndrome caused by a mechanism other than a SHP2 mutation), LEOPARD Syndrome (e.g., LEOPARD Syndrome caused by a mechanism other than a SHP2 mutation); tumors of hemopoietic and lymphoid system including myeloproliferative syndromes, myelodysplastic syndromes, and leukemia, e.g., acute myeloid leukemia, and juvenile myelomonocytic leukemias; esophageal cancer; breast cancer; lung cancer; colon cancer; gastric cancer, neuroblastoma, bladder cancer, prostate cancer; glioblastoma; urothelial carcinoma, uterine carcinoma, adenoid and ovarian sereous cystadenocarcinoma, paraganglioma, phaeochromocytoma, pancreatic cancer, adrenocortical carcinoma, stomach adenocarcinoma, sarcoma, rhabdomyosarcoma, lymphoma, head and neck cancer, skin cancer, peritoneum cancer, intestinal cancer (small and large intestine), thyroid cancer, endometrial cancer, cancer of the biliary tract, soft tissue cancer, ovarian cancer, central nervous system cancer (e.g., primary CNS lymphoma), stomach cancer, pituitary cancer, genital tract cancer, urinary tract cancer, salivary gland cancer, cervical cancer, liver cancer, eye cancer, cancer of the adrenal gland, cancer of autonomic ganglia, cancer of the upper aerodigestive tract, bone cancer, testicular cancer, pleura cancer, kidney cancer, penis cancer, parathyroid cancer, cancer of the meninges, vulvar cancer and melanoma comprising a method disclosed herein, such as, e.g., a monotherapy or combination therapy disclosed herein.

In various embodiments, the methods for treating such diseases or disorders involve administering to a subject an effective amount of a SHP2 inhibitor or a composition (e.g., a pharmaceutical composition) comprising a SHP2 inhibitor. Any compound or substance capable of inhibiting SHP2 may be utilized in application with the present disclosure to inhibit SHP2. Non-limiting examples of such SHP2 inhibitors are known in the art and are disclosed herein. For example, the compositions and methods described herein may utilize one or more SHP2 inhibitor selected from, but not limited to, any SHP2 inhibitor disclosed in Chen, Ying-Nan P et al., 148 Nature Vol 535 7 Jul. 2016, incorporated herein by reference in its entirety, including SHP099, disclosed therein. The compositions and methods described herein may utilize one or more SHP2 inhibitor selected from, but not limited to any SHP2 inhibitor disclosed in PCT application PCT/US2017/041577 (WO2018013597), which is incorporated herein by reference in its entirety. The compositions and methods described herein may utilize one or more SHP2 inhibitor selected from, but not limited to any SHP2 inhibitor disclosed in PCT applications PCT/IB2015/050343 (WO2015107493); PCT/IB2015/050344 (WO2015107494); PCT/IB2015/050345 (WO201507495); PCT/IB2016/053548 (WO2016/203404); PCT/IB2016/053549 (WO2016203405); PCT/IB2016/053550 (WO2016203406); PCT/US2010/045817 (WO2011022440); PCT/US2017/021784 (WO2017156397); and PCT/US2016/060787 (WO2017079723); and PCT/CN2017/087471 (WO 2017211303), each of which is incorporated herein by reference in its entirety. The compositions and methods described herein may utilize one or more SHP2 inhibitor selected from, but not limited to any SHP2 inhibitor disclosed in Chen L, et al., Mol Pharmacol. 2006 August; 70(2):562-70, incorporated herein by reference in its entirety, including NSC-87877 disclosed therein. The compositions and methods described herein may utilize TNO155, described under ClinicalTrials.gov Identifier: NCT03114319, available at world wide web address: clinicaltrials.gov/ct2/show/NCT03114319, incorporated herein by reference in its entirety. The compositions and methods described herein may utilize one or more SHP2 inhibitor selected from, but not limited to RMC-3943, disclosed herein; RMC-4550, disclosed herein; a SHP2 inhibitor compound of Formula I, Formula II, Formula III, Formula I-VI, Formula I-V2, Formula I-W, Formula I-X, Formula I-Y, Formula I-Z, Formula IV, Formula V, Formula VI, Formula IV-X, Formula IV-Y, Formula IV-Z, Formula VII, Formula VIII, Formula IX, and Formula X, disclosed herein; a compound from Table A1, disclosed herein; and a compound from Table A2, disclosed herein.

One aspect of the disclosure relates to compounds of Formula I:

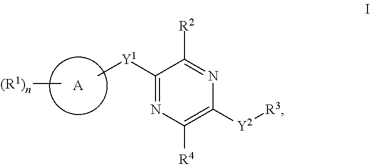

and pharmaceutically acceptable salts, prodrugs, solvates, hydrates, tautomers, or isomers thereof, wherein:

A is a 5- to 12-membered monocyclic or polycyclic cycloalkyl, heterocycloalkyl, aryl, or heteroaryl;

Y¹ is —S— or a direct bond;

Y² is —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a}), —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y², as drawn, is bound to the pyrazine ring and the bond on the right side of the Y² moiety is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence, —H, -D, —OH, —C₃-C₈cycloalkyl, or —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently, at each occurrence, —H, -D, —C₁-C₆alkyl, —C₃-C₈cycloalkyl, —C₂-C₆alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R³ is independently —C₁-C₆alkyl or a 3- to 12-membered monocyclic or polycyclic heterocycle, wherein each alkyl or heterocycle is optionally substituted with one or more —C₁-C₆alkyl, —OH, or —NH₂; or

R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆alkyl, —OH, or —NH₂;

R⁴ is independently —H, -D, or —C₁-C₆alkyl, wherein each alkyl is optionally substituted with one or more —OH, —NH₂, halogen, or oxo; or

R^{a} and R⁴, together with the atom or atoms to which they are attached, can combine to form a monocyclic or polycyclic C₃-C₁₂cycloalkyl or a monocyclic or polycyclic 3- to 12-membered heterocycle, wherein the cycloalkyl or heterocycle is optionally substituted with oxo;

R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, or —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently, at each occurrence, 1, 2, 3, 4, 5 or 6; and

n is independently, at each occurrence, 0, 1, 2, 3, 4, 5, 6, 7, 8, 9, or 10.

Another aspect of the disclosure relates to compounds of Formula II:

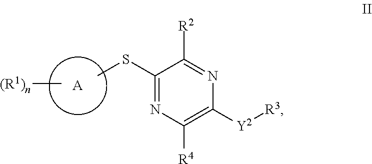

and pharmaceutically acceptable salts, prodrugs, solvates, hydrates, tautomers, or isomers thereof, wherein:

A is a 5- to 12-membered monocyclic or polycyclic cycloalkyl, heterocycloalkyl, aryl, or heteroaryl;

Y² is —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a}), —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y², as drawn, is bound to the pyrazine ring and the bond on the right side of the Y² moiety is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence, —H, -D, —OH, —C₃-C₈cycloalkyl, or —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently, at each occurrence, —H, -D, —C₁-C₆alkyl, —C₃-C₈cycloalkyl, —C₂-C₆alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R³ is independently —C₁-C₆alkyl or a 3- to 12-membered monocyclic or polycyclic heterocycle, wherein each alkyl or heterocycle is optionally substituted with one or more —C₁-C₆alkyl, —OH, or —NH₂; or

R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆alkyl, —OH, or —NH₂;

R⁴ is independently —H, -D, or —C₁-C₆alkyl, wherein each alkyl is optionally substituted with one or more —OH, —NH₂, halogen, or oxo; or

R^{a} and R⁴, together with the atom or atoms to which they are attached, can combine to form a monocyclic or polycyclic C₃-C₁₂cycloalkyl or a monocyclic or polycyclic 3- to 12-membered heterocycle, wherein the cycloalkyl or heterocycle is optionally substituted with oxo;

R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, or —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently, at each occurrence, 1, 2, 3, 4, 5 or 6; and

n is independently, at each occurrence, 0, 1, 2, 3, 4, 5, 6, 7, 8, 9, or 10.

Another aspect of the disclosure relates to compounds of Formula III:

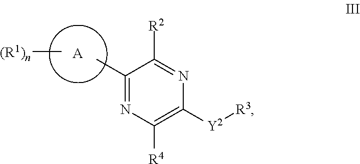

and pharmaceutically acceptable salts, prodrugs, solvates, hydrates, tautomers, or isomers thereof, wherein:

A is a 5- to 12-membered monocyclic or polycyclic cycloalkyl, heterocycloalkyl, aryl, or heteroaryl;

Y² is —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y², as drawn, is bound to the pyrazine ring and the bond on the right side of the Y² moiety is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence, —H, -D, —OH, —C₃-C₈cycloalkyl, or —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently, at each occurrence, —H, -D, —C₁-C₆alkyl, —C₃-C₈cycloalkyl, —C₂-C₆alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R³ is independently —C₁-C₆alkyl or a 3- to 12-membered monocyclic or polycyclic heterocycle, wherein each alkyl or heterocycle is optionally substituted with one or more —C₁-C₆alkyl, —OH, or —NH₂; or

R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆alkyl, —OH, or —NH₂;

R⁴ is independently —H, -D, or —C₁-C₆alkyl, wherein each alkyl is optionally substituted with one or more —OH, —NH₂, halogen, or oxo; or

R^{a} and R⁴, together with the atom or atoms to which they are attached, can combine to form a monocyclic or polycyclic C₃-C₁₂cycloalkyl or a monocyclic or polycyclic 3- to 12-membered heterocycle, wherein the cycloalkyl or heterocycle is optionally substituted with oxo;

R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, or —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently, at each occurrence, 1, 2, 3, 4, 5 or 6; and

n is independently, at each occurrence, 0, 1, 2, 3, 4, 5, 6, 7, 8, 9, or 10.

One aspect of the disclosure related to compounds of Formula I-VI:

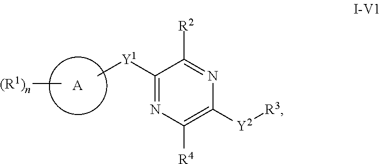

and pharmaceutically acceptable salts, prodrugs, solvates, hydrates, tautomers, or isomers thereof, wherein:

A is cycloalkyl, heterocycloalkyl, aryl, or heteroaryl, wherein cycloalkyl, heterocycloalkyl, aryl, and heteroaryl are 5- to 12-membered monocyclic or 5- to 12-membered polycyclic;

Y¹ is —S—, a direct bond, —NH—, —S(O)₂—, —S(O)₂—NH—, —C(═CH₂)—, —CH—, or —S(O)—;

Y² is —NR^{a}—, wherein the bond on the left side of Y², as drawn, is bound to the pyrazine ring and the bond on the right side of the Y² moiety, as drawn, is bound to R³;

R^{a} and R⁴, together with the atom or atoms to which they are attached, are combined to form a monocyclic or polycyclic C₃-C₁₂cycloalkyl or a monocyclic or polycyclic 3- to 12-membered heterocycle, wherein the cycloalkyl or heterocycle is optionally substituted with oxo; wherein the heterocycle optionally comprises —S(O)₂— in the heterocycle;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, —OR⁶, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, —CO₂R⁵, —C(O)NR⁵R⁶, —NR⁵C(O)R⁶, monocyclic or polycyclic heterocyclyl, spiroheterocyclyl, heteroaryl, or oxo, wherein each alkyl, alkenyl, cycloalkenyl, alkynyl, cycloalkyl, heterocyclyl, spiroheterocyclyl, or heteroaryl is optionally substituted with one or more —OH, halogen, —NO₂, oxo, ═O, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —NH₂, —OR^{b}, —CN, —C₁-C₆alkyl, —C₂-C₆alkenyl, —C₄-C₈cycloalkenyl, —C₂-C₆alkynyl, halogen, —C(O)OR^{b}, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{b} is independently, at each occurrence, —H, -D, —OH, —C₁-C₆alkyl, —C₃-C₈cycloalkyl, —C₂-C₆alkenyl, —(CH₂)ₙ-aryl, heterocyclyl containing 1-5 heteroatoms selected from the group consisting of N, S, P, and O, or heteroaryl containing 1-5 heteroatoms selected from the group consisting of N, S, P, and O; wherein each alkyl, cycloalkyl, alkenyl, heterocycle, heteroaryl, or —(CH₂)n-aryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)NR⁵R⁶, —NR⁵C(O)R⁶, heterocycle, aryl, heteroaryl, —(CH₂)ₙOH, —C₁-C₆alkyl, —CF₃, —CHF₂, or —CH₂F;

R³ is independently —H, —C₁-C₆alkyl, a 3- to 12-membered monocyclic or polycyclic heterocycle, a 5- to 12-membered spiroheterocycle, —C₃-C₈cycloalkyl, or —(CH₂)ₙ—R^{b}, wherein each alkyl, spiroheterocycle, heterocycle, or cycloalkyl is optionally substituted with one or more —C₁-C₆alkyl, —OH, —NH₂, —OR^{b}, —NHR^{b}, —(CH₂)ₙOH, heterocyclyl, or spiroheterocyclyl;

R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, —CF₃, or —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OR, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN; and

n is independently, at each occurrence, 0, 1, 2, 3, 4, 5, 6, 7, 8, 9, or 10.

One aspect of the disclosure related to compounds of Formula I-V2:

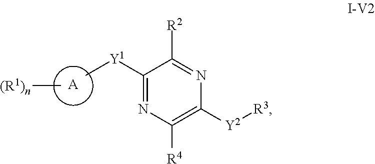

and pharmaceutically acceptable salts, prodrugs, solvates, hydrates, tautomers, and isomers thereof, wherein:

A is cycloalkyl, heterocycloalkyl, aryl, or heteroaryl, wherein cycloalkyl, heterocycloalkyl, aryl, and heteroaryl are 5- to 12-membered monocyclic or 5- to 12-membered polycyclic;

Y¹ is —S—, a direct bond, —NH—, —S(O)₂—, —S(O)₂—NH—, —C(═CH₂)—, —CH—, or —S(O)—;

Y² is —NR^{a}—, wherein the bond on the left side of Y², as drawn, is bound to the pyrazine ring and the bond on the right side of the Y² moiety, as drawn, is bound to R³;

R³ is combined with R^{a} to form a 3- to 12-membered polycyclic heterocycle or a 5- to 12-membered spiroheterocycle, wherein each heterocycle or spiroheterocycle is optionally substituted with one or more —C₁-C₆alkyl, halogen, —OH, —OR^{b}, —NH₂, —NHR^{b}, heteroaryl, heterocyclyl, —(CH₂)ₙNH₂, —(CH₂)ₙOH, —COOR^{b}, —CONHR^{b}, —CONH(CH₂)ₙCOOR^{b}, —NHCOOR^{b}, —CF₃, —CHF₂, —CH₂F, or ═O;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, —OR⁶, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, —CO₂R⁵, —C(O)NR⁵R⁶, —NR⁵C(O)R⁶, monocyclic or polycyclic heterocyclyl, spiroheterocyclyl, heteroaryl, or oxo, wherein each alkyl, alkenyl, cycloalkenyl, alkynyl, cycloalkyl, heterocyclyl, spiroheterocyclyl, or heteroaryl is optionally substituted with one or more —OH, halogen, —NO₂, oxo, ═O, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —NH₂, —OR^{b}, —CN, —C₁-C₆alkyl, —C₂-C₆alkenyl, —C₄-C₈cycloalkenyl, —C₂-C₆alkynyl, halogen, —C(O)OR^{b}, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{b} is independently, at each occurrence, —H, -D, —OH, —C₁-C₆alkyl, —C₃-C₈cycloalkyl, —C₂-C₆alkenyl, —(CH₂)n-aryl, heterocyclyl containing 1-5 heteroatoms selected from the group consisting of N, S, P, and O, or heteroaryl containing 1-5 heteroatoms selected from the group consisting of N, S, P, and O; wherein each alkyl, cycloalkyl, alkenyl, heterocycle, heteroaryl, or —(CH₂)n-aryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)NR⁵R⁶, —NR⁵C(O)R⁶, heterocycle, aryl, heteroaryl, —(CH₂)ₙOH, —C₁-C₆alkyl, —CF₃, —CHF₂, or —CH₂F;

R⁴ is independently —H, -D, —C₁-C₆alkyl, —C₁-C₆haloalkyl, —C₁-C₆hydroxyalkyl, —CF₂OH, —CHFOH, —NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂₀H, —C(O)OR⁵, —NH(CH₂)ₙOH, —C(O)NH(CH₂)ₙOH, —C(O)NH(CH₂)ₙR^{b}, —C(O)R^{b}, —NH₂, —OH, —CN, —C(O)NR⁵R⁶, —S(O)₂NR⁵R⁶, C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O, wherein each alkyl, cycloalkyl, or heterocyclyl is optionally substituted with one or more —OH, —NH₂, —OR^{b}, halogen, or oxo; wherein each aryl or heteroaryl is optionally substituted with one or more —OH, —NH₂, or halogen;

R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, —CF₃, or —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OR, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN; and

n is independently, at each occurrence, 0, 1, 2, 3, 4, 5, 6, 7, 8, 9, or 10.

One aspect of the disclosure relates to compounds of Formula I-W:

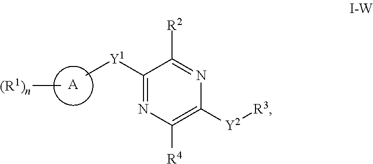

and pharmaceutically acceptable salts, prodrugs, solvates, hydrates, tautomers, and isomers thereof, wherein:

A is cycloalkyl, heterocycloalkyl, aryl, or heteroaryl, wherein cycloalkyl, heterocycloalkyl, aryl, and heteroaryl are 5- to 12-membered monocyclic or 5- to 12-membered polycyclic;

Y¹ is —S—, a direct bond, —NH—, —S(O)₂—, —S(O)₂—NH—, —C(═CH₂)—, —CH—, or —S(O)—;

Y² is —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a}), —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y², as drawn, is bound to the pyrazine ring and the bond on the right side of the Y² moiety, as drawn, is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, —OR⁶, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, —CO₂R⁵, —C(O)NR⁵R⁶, —NR⁵C(O)R⁶, monocyclic or polycyclic heterocyclyl, spiroheterocyclyl, heteroaryl, or oxo, wherein each alkyl, alkenyl, cycloalkenyl, alkynyl, cycloalkyl, heterocyclyl, spiroheterocyclyl, or heteroaryl is optionally substituted with one or more —OH, halogen, —NO₂, oxo, ═O, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —CN, —C₁-C₆alkyl, —C₂-C₆alkenyl, —C₄-C₈cycloalkenyl, —C₂-C₆alkynyl, halogen, —C(O)OR^{b}, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence, —H, -D, —OH, —C₃-C₈cycloalkyl, —C₁-C₆alkyl, 3- to 12-membered heterocyclyl, or —(CH₂)ₙ-aryl, wherein each alkyl or cycloalkyl is optionally substituted with one or more —NH₂, or wherein 2 R^{a}, together with the carbon atom to which they are both attached, can combine to form a 3- to 8-membered cycloalkyl;

R^{b} is independently, at each occurrence, —H, -D, —OH, —C₁-C₆alkyl, —C₃-C₈cycloalkyl, —C₂-C₆alkenyl, —(CH₂)n-aryl, heterocyclyl containing 1-5 heteroatoms selected from the group consisting of N, S, P, and O, or heteroaryl containing 1-5 heteroatoms selected from the group consisting of N, S, P, and O; wherein each alkyl, cycloalkyl, alkenyl, heterocycle, heteroaryl, or —(CH₂)n-aryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)NR⁵R⁶, —NR⁵C(O)R⁶, heterocycle, aryl, heteroaryl, —(CH₂)ₙOH, —C₁-C₆alkyl, —CF₃, —CHF₂, or —CH₂F;

R³ is independently —H, —C₁-C₆alkyl, a 3- to 12-membered monocyclic or polycyclic heterocycle, a 5- to 12-membered spiroheterocycle, C₃-C₈cycloalkyl, or —(CH₂)ₙ—R^{b}, wherein each alkyl, spiroheterocycle, heterocycle, or cycloalkyl is optionally substituted with one or more —C₁-C₆alkyl, —OH, —NH₂, —OR^{b}, —NHR^{b}, —(CH₂)ₙOH, heterocyclyl, or spiroheterocyclyl; or

R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆alkyl, halogen, —OH, —OR^{b}, —NH₂, —NHR^{b}, heteroaryl, heterocyclyl, —(CH₂)ₙNH₂, —(CH₂)ₙOH, —COOR^{b}, —CONHR^{b}, —CONH(CH₂)ₙCOOR^{b}, —NHCOOR^{b}, —CF₃, —CHF₂, —CH₂F, or ═O;

R⁴ is independently —H, -D, —C₁-C₆alkyl, —C₁-C₆haloalkyl, —C₁-C₆hydroxyalkyl —CF₂OH, —CHFOH —NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂₀H, —C(O)OR⁵, —NH(CH₂)ₙOH, —C(O)NH(CH₂)ₙOH, —C(O)NH(CH₂)ₙR^{b}, —C(O)R^{b}, —NH₂, —OH, —CN, —C(O)NR⁵R⁶, —S(O)₂NR⁵R⁶, C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O, wherein each alkyl, cycloalkyl, or heterocyclyl is optionally substituted with one or more —OH, —NH₂, —OR^{b}, halogen, or oxo; wherein each aryl or heteroaryl is optionally substituted with one or more —OH, —NH₂, or halogen; or

R^{a} and R⁴, together with the atom or atoms to which they are attached, can combine to form a monocyclic or polycyclic C₃-C₁₂cycloalkyl or a monocyclic or polycyclic 3- to 12-membered heterocycle, wherein the cycloalkyl or heterocycle is optionally substituted with oxo; wherein the heterocycle optionally comprises —S(O)₂— in the heterocycle;

R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, —CF₃, or —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OR, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently, at each occurrence, 1, 2, 3, 4, 5 or 6; and

n is independently, at each occurrence, 0, 1, 2, 3, 4, 5, 6, 7, 8, 9, or 10.

One aspect of the disclosure relates to compounds of Formula I-X:

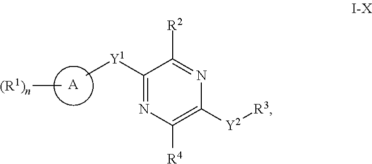

and pharmaceutically acceptable salts, prodrugs, solvates, hydrates, tautomers, or isomers thereof, wherein:

A is a 5- to 12-membered monocyclic or polycyclic cycloalkyl, heterocycloalkyl, aryl, or heteroaryl;

Y¹ is —S— or a direct bond;

Y² is —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a}), —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y², as drawn, is bound to the pyrazine ring and the bond on the right side of the Y² moiety, as drawn, is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence, —H, -D, —OH, —C₃-C₈cycloalkyl, or —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently, at each occurrence, —H, -D, —C₁-C₆alkyl, —C₃-C₈cycloalkyl, —C₂-C₆alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R³ is independently —H, —C₁-C₆alkyl, or a 3- to 12-membered monocyclic or polycyclic heterocycle, wherein each alkyl or heterocycle is optionally substituted with one or more —C₁-C₆alkyl, —OH, or —NH₂; or

R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆alkyl, —OH, or —NH₂;

R⁴ is independently —H, -D, —C₁-C₆alkyl, —NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂₀H, —C(O)OR⁵, —C(O)NR⁵R⁶, —S(O)₂NR⁵R⁶, C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O, wherein each alkyl, cycloalkyl, or heterocyclyl is optionally substituted with one or more —OH, —NH₂, halogen, or oxo; wherein each aryl or heteroaryl is optionally substituted with one or more —OH, —NH₂, or halogen; or

R^{a} and R⁴, together with the atom or atoms to which they are attached, can combine to form a monocyclic or polycyclic C₃-C₁₂cycloalkyl or a monocyclic or polycyclic 3- to 12-membered heterocycle, wherein the cycloalkyl or heterocycle is optionally substituted with oxo; wherein the heterocycle optionally comprises —S(O)₂— in the heterocycle;

R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, or —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently, at each occurrence, 1, 2, 3, 4, 5 or 6; and

n is independently, at each occurrence, 0, 1, 2, 3, 4, 5, 6, 7, 8, 9, or 10.

One aspect of the disclosure relates to compounds of Formula I-Y:

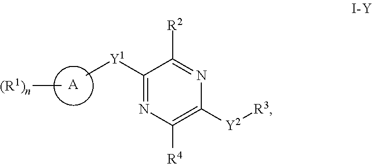

and pharmaceutically acceptable salts, prodrugs, solvates, hydrates, tautomers, or isomers thereof, wherein:

A is a 5- to 12-membered monocyclic or polycyclic cycloalkyl, heterocycloalkyl, aryl, or heteroaryl;

Y¹ is —S— or a direct bond;

Y² is —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a}), —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, or —OC(O)O—; wherein the bond on the left side of Y², as drawn, is bound to the pyrazine ring and the bond on the right side of the Y² moiety, as drawn, is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence, —H, -D, —OH, —C₃-C₈cycloalkyl, or —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently, at each occurrence, —H, -D, —C₁-C₆alkyl, —C₃-C₈cycloalkyl, —C₂-C₆alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆alkyl, —CF₃, —CHF₂, or —CH₂F;

R³ is independently —H, —C₁-C₆alkyl, a 3- to 12-membered monocyclic or polycyclic heterocycle, C₃-C₈cycloalkyl, or —(CH₂)ₙ—R, wherein each alkyl, heterocycle, or cycloalkyl is optionally substituted with one or more —C₁-C₆alkyl, —OH, —NH₂, —OR^{b}, —NHR^{b}, —(CH₂)ₙOH, heterocyclyl, or spiroheterocyclyl; or

R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆alkyl, —OH, —NH₂, heteroaryl, heterocyclyl, —(CH₂)ₙNH₂, —COOR^{b}, —CONHR^{b}, —CONH(CH₂)ₙCOOR^{b}, —NHCOOR^{b}, —CF₃, —CHF₂, or —CH₂F;

R⁴ is independently —H, -D, —C₁-C₆alkyl, —NH—NHR⁵, —NH—OR^{b}, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂₀H, —C(O)OR⁵, —NH(CH₂)ₙOH, —C(O)NH(CH₂)ₙOH, —C(O)NH(CH₂)ₙR^{b}, —C(O)R^{b}, —NH₂, —OH, —CN, —C(O)NR⁵R⁶, —S(O)₂NR⁵R⁶, C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O, wherein each alkyl, cycloalkyl, or heterocyclyl is optionally substituted with one or more —OH, —NH₂, halogen, or oxo; wherein each aryl or heteroaryl is optionally substituted with one or more —OH, —NH₂, or halogen; or

R^{a} and R⁴, together with the atom or atoms to which they are attached, can combine to form a monocyclic or polycyclic C₃-C₁₂cycloalkyl or a monocyclic or polycyclic 3- to 12-membered heterocycle, wherein the cycloalkyl or heterocycle is optionally substituted with oxo; wherein the heterocycle optionally comprises —S(O)₂— in the heterocycle;

R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, or —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently, at each occurrence, 1, 2, 3, 4, 5 or 6; and

n is independently, at each occurrence, 0, 1, 2, 3, 4, 5, 6, 7, 8, 9, or 10.

One aspect of the disclosure relates to compounds of Formula I-Z:

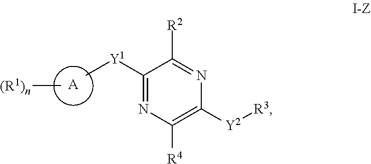

and pharmaceutically acceptable salts, prodrugs, solvates, hydrates, tautomers, or isomers thereof, wherein:

A is a 5- to 12-membered monocyclic or polycyclic cycloalkyl, heterocycloalkyl, aryl, or heteroaryl;

Y¹ is —S—, a direct bond, —NH—, —S(O)₂—, —S(O)₂—NH—, —C(═CH₂)—, —CH—, or —S(O)—;

Y² is —NR^{a}—, —(CR^{a}₂)ₘ—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —OC(O)N(R^{a})—, —N(R^{a})C(O)O—, —C(O)N(R^{a})O—, —N(R^{a})C(S)—, or —C(S)N(R^{a})—; wherein the bond on the left side of Y², as drawn, is bound to the pyrazine ring and the bond on the right side of the Y² moiety, as drawn, is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —NH₂, —CN, —C₁-C₆alkyl, —C₂-C₆alkenyl, —C₄-C₈cycloalkenyl, —C₂-C₆alkynyl, halogen, —C(O)OR^{b}, —C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence —OH, —C₃-C₈cycloalkyl, or —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently, at each occurrence, —H, -D, —C₁-C₆alkyl, —C₃-C₈cycloalkyl, —C₂-C₆alkenyl, or heterocyclyl containing 1-5 heteroatoms selected from the group consisting of N, S, P, and O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆alkyl, —CF₃, —CHF₂, or —CH₂F;

R³ is independently —H, —C₁-C₆alkyl, a 3- to 12-membered monocyclic or polycyclic heterocycle, C₃-C₈cycloalkyl, or —(CH₂)ₙ—R, wherein each alkyl, heterocycle, or cycloalkyl is optionally substituted with one or more —C₁-C₆alkyl, —OH, —NH₂, —OR^{b}, —NHR^{b}, —(CH₂)ₙOH, heterocyclyl, or spiroheterocyclyl; or

R³ can combine with R^{a} to form a 3- to 12-membered monocyclic or polycyclic heterocycle or a 5- to 12-membered spiroheterocycle, wherein each heterocycle or spiroheterocycle is optionally substituted with one or more —C₁-C₆alkyl, —OH, —NH₂, heteroaryl, heterocyclyl, —(CH₂)ₙNH₂, —COOR^{b}, —CONHR^{b}, —CONH(CH₂)ₙCOOR^{b}, —NHCOOR^{b}, —CF₃, —CHF₂, or —CH₂F;

R⁴ is independently —C₁-C₆alkyl, —NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂₀H, —C(O)OR⁵, —NH(CH₂)ₙOH, —C(O)NH(CH₂)ₙOH, —C(O)NH(CH₂)ₙR^{b}, —C(O)R^{b}, —NH₂, —OH, —C(O)NR⁵R⁶, —S(O)₂NR⁵R⁶, C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O, wherein each alkyl, cycloalkyl, or heterocyclyl is optionally substituted with one or more —OH, —NH₂, halogen, or oxo; wherein each aryl or heteroaryl is optionally substituted with one or more —OH, —NH₂, or halogen;

R^{a} and R⁴, together with the atom or atoms to which they are attached, are combined to form a monocyclic or polycyclic C₃-C₁₂cycloalkyl or a monocyclic or polycyclic 3- to 12-membered heterocycle, wherein the cycloalkyl or heterocycle is optionally substituted with oxo; wherein the heterocycle optionally comprises —S(O)₂— in the heterocycle;

R⁵ and R⁶ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, or —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, or a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently, at each occurrence, 1, 2, 3, 4, 5 or 6; and

n is independently, at each occurrence, 0, 1, 2, 3, 4, 5, 6, 7, 8, 9, or 10.

One aspect of the invention relates to compounds of Formula IV:

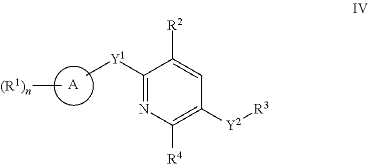

and pharmaceutically acceptable salts, prodrugs, solvates, hydrates, tautomers, or isomers thereof, wherein:

A is selected from the group consisting of 5- to 12-membered monocyclic or polycyclic cycloalkyl, heterocycloalkyl, aryl, or heteroaryl;

Y¹ is —S— or a direct bond;

Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the pyridine ring and the bond on the right side of the Y² moiety is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —R⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently —H, -D, —C₁-C₆alkyl, —C₁-C₆cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; R³ is independently, at each occurrence, selected from the group consisting of —C₁-C₆alkyl, or a 3- to 12-membered monocyclic or polycyclic heterocycle, wherein each alkyl or heterocycle is optionally substituted with one or more —C₁-C₆alkyl, —OH, or —NH₂; or

R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, or —NH₂;

R⁴ is independently, at each occurrence, —H, -D, or —C₁-C₆alkyl, wherein each alkyl is optionally substituted with one or more —OH, —NH₂, halogen, or oxo; or

R^{a} and R⁴, together with the atom or atoms to which they are attached, can combine to form a monocyclic or polycyclic C₃-C₁₂cycloalkyl, or a monocyclic or polycyclic 3- to 12-membered heterocycle, wherein the cycloalkyl or heterocycle is optionally substituted with oxo;

R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently 1, 2, 3, 4, 5 or 6; and

n is independently 0, 1, 2, 3, 4, 5, 6, 7, 8, 9, or 10.

Another aspect of the invention relates to compounds of Formula V:

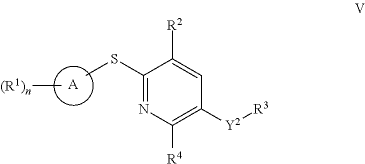

and pharmaceutically acceptable salts, prodrugs, solvates, hydrates, tautomers, or isomers thereof, wherein:

A is selected from the group consisting of 5- to 12-membered monocyclic or polycyclic cycloalkyl, heterocycloalkyl, aryl, or heteroaryl;

Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the pyridine ring and the bond on the right side of the Y² moiety is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —R⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently —H, -D, —C₁-C₆alkyl, —C₁-C₆cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R³ is independently, at each occurrence, selected from the group consisting of —C₁-C₆alkyl, or a 3- to 12-membered monocyclic or polycyclic heterocycle, wherein each alkyl or heterocycle is optionally substituted with one or more —C₁-C₆alkyl, —OH, or —NH₂; or

R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, or —NH₂;

R⁴ is independently, at each occurrence, —H, -D, or —C₁-C₆alkyl, wherein each alkyl is optionally substituted with one or more —OH, —NH₂, halogen, or oxo; or

R^{a} and R⁴, together with the atom or atoms to which they are attached, can combine to form a monocyclic or polycyclic C₃-C₁₂cycloalkyl, or a monocyclic or polycyclic 3- to 12-membered heterocycle, wherein the cycloalkyl or heterocycle is optionally substituted with oxo;

R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently 1, 2, 3, 4, 5 or 6; and

n is independently 0, 1, 2, 3, 4, 5, 6, 7, 8, 9, or 10.

Another aspect of the invention relates to compounds of Formula VI:

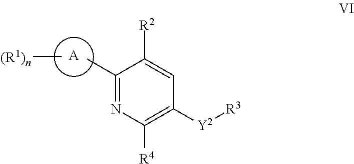

and pharmaceutically acceptable salts, prodrugs, solvates, hydrates, tautomers, or isomers thereof, wherein:

A is selected from the group consisting of 5- to 12-membered monocyclic or polycyclic cycloalkyl, heterocycloalkyl, aryl, or heteroaryl;

Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the pyridine ring and the bond on the right side of the Y² moiety is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —R⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently —H, -D, —C₁-C₆alkyl, —C₁-C₆cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R³ is independently, at each occurrence, selected from the group consisting of —C₁-C₆alkyl, or a 3- to 12-membered monocyclic or polycyclic heterocycle, wherein each alkyl or heterocycle is optionally substituted with one or more —C₁-C₆alkyl, —OH, or —NH₂; or

R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, or —NH₂;

R⁴ is independently, at each occurrence, —H, -D, or —C₁-C₆alkyl, wherein each alkyl is optionally substituted with one or more —OH, —NH₂, halogen, or oxo; or

R^{a} and R⁴, together with the atom or atoms to which they are attached, can combine to form a monocyclic or polycyclic C₃-C₁₂cycloalkyl, or a monocyclic or polycyclic 3- to 12-membered heterocycle, wherein the cycloalkyl or heterocycle is optionally substituted with oxo;

R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently 1, 2, 3, 4, 5 or 6; and

n is independently 0, 1, 2, 3, 4, 5, 6, 7, 8, 9, or 10.

One aspect of the invention relates to compounds of Formula IV-Y:

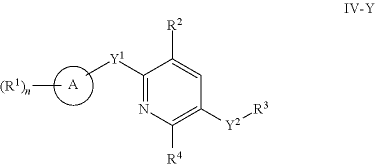

or a pharmaceutically acceptable salt, prodrug, solvate, hydrate, tautomer, or isomer thereof, wherein:

A is selected from the group consisting of 5- to 12-membered monocyclic or polycyclic cycloalkyl, heterocycloalkyl, aryl, or heteroaryl;

Y¹ is —S— or a direct bond;

Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the pyridine ring and the bond on the right side of the Y² moiety, as drawn, is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —CN, —C₁-C₆alkyl, —C₂-C₆alkenyl, —C₄-C₈cycloalkenyl, —C₂-C₆alkynyl, —C₃-C₈cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently —H, -D, —C₁-C₆alkyl, —C₁-C₆cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —R⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆alkyl, CF₃, CHF₂, or CH₂F;

R³ is independently, at each occurrence, selected from the group consisting of —H, —C₁-C₆alkyl, a 3- to 12-membered monocyclic or polycyclic heterocycle, C₃-C₈cycloalkyl, or —(CH₂)ₙ—R, wherein each alkyl, heterocycle, or cycloalkyl is optionally substituted with one or more —C₁-C₆alkyl, —OH, —NH₂, —OR^{a}, —NHR^{a}, —(CH₂)ₙOH, heterocyclyl, or spiroheterocyclyl; or

R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, —NH₂, heteroaryl, heterocyclyl, —(CH₂)ₙNH₂, —COOR^{a}, —CONHR^{b}, —CONH(CH₂)ₙCOOR^{a}, —NHCOOR^{a}, —CF₃, CHF₂, or CH₂F;

R⁴ is independently, at each occurrence, —H, -D, —C₁-C₆alkyl, —NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂₀H, —C(O)OR⁵, —NH(CH₂)ₙOH, —C(O)NH(CH₂)ₙOH, —C(O)NH(CH₂)ₙR^{b}, —C(O)R^{b}, NH₂, —OH, —CN, —C(O)NR⁵R⁶, —S(O)₂NR⁵R⁶, C₃-C₈cycloalkyl, aryl, heterocyclyl containing 1-5 heteroatoms selected from the group consisting of N, S, P, or O, heteroaryl containing 1-5 heteroatoms selected from the group consisting of N, S, P, or O, wherein each alkyl, cycloalkyl, or heterocyclyl is optionally substituted with one or more —OH, —NH₂, halogen, or oxo; wherein each aryl or heteroaryl is optionally substituted with one or more —OH, —NH₂, or halogen; or

R^{a} and R⁴, together with the atom or atoms to which they are attached, can combine to form a monocyclic or polycyclic C₃-C₁₂cycloalkyl, or a monocyclic or polycyclic 3- to 12-membered heterocycle, wherein the cycloalkyl or heterocycle is optionally substituted with oxo; wherein the heterocycle optionally comprises —S(O)₂— in the heterocycle;

R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently 1, 2, 3, 4, 5 or 6; and

n is independently 0, 1, 2, 3, 4, 5, 6, 7, 8, 9, or 10.

One aspect of the invention relates to compounds of Formula IV-Z:

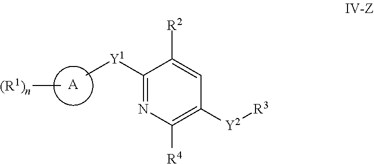

or a pharmaceutically acceptable salt, prodrug, solvate, hydrate, tautomer, or isomer thereof, wherein:

A is selected from the group consisting of 5- to 12-membered monocyclic or polycyclic cycloalkyl, heterocycloalkyl, aryl, or heteroaryl;

Y¹ is —S—, a direct bond, —NH—, —S(O)₂—, —S(O)₂—NH—, —C(═CH₂)—, —CH—, or —S(O)—;

Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the pyridine ring and the bond on the right side of the Y² moiety, as drawn, is bound to R³;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

R² is independently —OR^{b}, —CN, —C₁-C₆alkyl, —C₂-C₆alkenyl, —C₄-C₈cycloalkenyl, —C₂-C₆alkynyl, —NH₂, halogen, —C(O)OR^{a}, —C₃-C₈cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently —H, -D, —C₁-C₆alkyl, —C₁-C₆cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —R⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆alkyl, CF₃, CHF₂, or CH₂F;

R³ is independently, at each occurrence, selected from the group consisting of —H, —C₁-C₆alkyl, a 3- to 12-membered monocyclic or polycyclic heterocycle, C₃-C₈cycloalkyl, or —(CH₂)ₙ—R, wherein each alkyl, heterocycle, or cycloalkyl is optionally substituted with one or more —C₁-C₆alkyl, —OH, —NH₂, —OR^{a}, —NHR^{a}, —(CH₂)ₙOH, heterocyclyl, or spiroheterocyclyl; or

R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, —NH₂, heteroaryl, heterocyclyl, —(CH₂)ₙNH₂, —COOR^{a}, —CONHR^{b}, —CONH(CH₂)ₙCOOR^{a}, —NHCOOR^{a}, —CF₃, CHF₂, or CH₂F;

R⁴ is independently, at each occurrence, —H, -D, —C₁-C₆alkyl, —NH—NHR⁵, —NH—OR⁵, —O—NR⁵R⁶, —NHR⁵, —OR⁵, —NHC(O)R⁵, —NHC(O)NHR⁵, —NHS(O)₂R⁵, —NHS(O)₂NHR⁵, —S(O)₂₀H, —C(O)OR⁵, —NH(CH₂)ₙOH, —C(O)NH(CH₂)ₙOH, —C(O)NH(CH₂)ₙR^{b}, —C(O)R^{b}, NH₂, —OH, —CN, —C(O)NR⁵R⁶, —S(O)₂NR⁵R⁶, C₃-C₈cycloalkyl, aryl, heterocyclyl containing 1-5 heteroatoms selected from the group consisting of N, S, P, or O, heteroaryl containing 1-5 heteroatoms selected from the group consisting of N, S, P, or O, wherein each alkyl, cycloalkyl, or heterocyclyl is optionally substituted with one or more —OH, —NH₂, halogen, or oxo; wherein each aryl or heteroaryl is optionally substituted with one or more —OH, —NH₂, or halogen; or

R^{a} and R⁴, together with the atom or atoms to which they are attached, can combine to form a monocyclic or polycyclic C₃-C₁₂cycloalkyl, or a monocyclic or polycyclic 3- to 12-membered heterocycle, wherein the cycloalkyl or heterocycle is optionally substituted with oxo; wherein the heterocycle optionally comprises —S(O)₂— in the heterocycle; R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently 1, 2, 3, 4, 5 or 6; and

n is independently 0, 1, 2, 3, 4, 5, 6, 7, 8, 9, or 10.

One aspect of the invention relates to compounds of Formula VII:

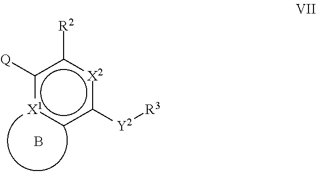

and pharmaceutically acceptable salts, prodrugs, solvates, hydrates, tautomers, or isomers thereof, wherein:

Q is H or

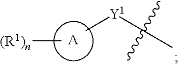

A is selected from the group consisting of 5- to 12-membered monocyclic or polycyclic cycloalkyl, heterocycloalkyl, aryl, or heteroaryl;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

Y¹ is —S—, a direct bond, —NH—, —S(O)₂—, —S(O)₂—NH—, —C(═CH₂)—, —CH—, or —S(O)—;

X¹ is N or C;

X² is N or CH;

B, including the atoms at the points of attachment, is a monocyclic or polycyclic 5- to 12-membered heterocycle or a monocyclic or polycyclic 5- to 12-membered heteroaryl;

R² is independently H, —OR^{b}, —NR⁵R⁶, —CN, —C₁-C₆alkyl, —C₂-C₆alkenyl, —C₄-C₈cycloalkenyl, —C₂-C₆alkynyl, —NH₂, halogen, —C(O)OR^{a}, —C₃-C₈cycloalkyl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the ring and the bond on the right side of the Y² moiety, as drawn, is bound to R³;

R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently —H, -D, —C₁-C₆alkyl, —C₁-C₆cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —R⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆alkyl, CF₃, CHF₂, or CH₂F;

R³ is independently, at each occurrence, selected from the group consisting of —H, —C₁-C₆alkyl, a 3- to 12-membered monocyclic or polycyclic heterocycle, C₃-C₈cycloalkyl, or —(CH₂)ₙ—R, wherein each alkyl, heterocycle, or cycloalkyl is optionally substituted with one or more —C₁-C₆alkyl, —OH, —NH₂, —OR^{a}, —NHR^{a}, —(CH₂)ₙOH, heterocyclyl, or spiroheterocyclyl; or

R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, —NH₂, heteroaryl, heterocyclyl, —(CH₂)ₙNH₂, —COOR^{a}, —CONHR^{b}, —CONH(CH₂)ₙCOOR^{a}, —NHCOOR^{a}, —CF₃, CHF₂, or CH₂F;

R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently 1, 2, 3, 4, 5 or 6; and

n is independently 0, 1, 2, 3, 4, 5, 6, 7, 8, 9, or 10.

Another aspect of the invention relates to compounds of Formula VIII:

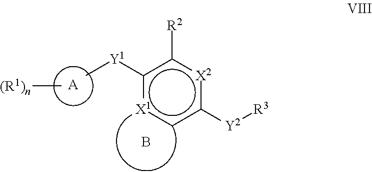

and pharmaceutically acceptable salts, prodrugs, solvates, hydrates, tautomers, or isomers thereof, wherein:

A is selected from the group consisting of 5- to 12-membered monocyclic or polycyclic cycloalkyl, heterocycloalkyl, aryl, or heteroaryl;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

Y¹ is —S—, a direct bond, —NH—, —S(O)₂—, —S(O)₂—NH—, —C(═CH₂)—, —CH—, or —S(O)—;

X¹ is N or C;

X² is N or CH;

B, including the atoms at the points of attachment, is a monocyclic or polycyclic 5- to 12-membered heterocycle or a monocyclic or polycyclic 5- to 12-membered heteroaryl;

R² is independently H, —OR^{b}, —NR⁵R⁶, —CN, —C₁-C₆alkyl, —C₂-C₆alkenyl, —C₄-C₈cycloalkenyl, —C₂-C₆alkynyl, —NH₂, halogen, —C(O)OR^{a}, —C₃-C₈cycloalkyl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the ring and the bond on the right side of the Y² moiety, as drawn, is bound to R³;

R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently —H, -D, —C₁-C₆alkyl, —C₁-C₆cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆alkyl, CF₃, CHF₂, or CH₂F;

R³ is independently, at each occurrence, selected from the group consisting of —H, —C₁-C₆alkyl, a 3- to 12-membered monocyclic or polycyclic heterocycle, C₃-C₈cycloalkyl, or —(CH₂)ₙ—R, wherein each alkyl, heterocycle, or cycloalkyl is optionally substituted with one or more —C₁-C₆alkyl, —OH, —NH₂, —OR^{a}, —NHR^{a}, —(CH₂)ₙOH, heterocyclyl, or spiroheterocyclyl; or

R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, —NH₂, heteroaryl, heterocyclyl, —(CH₂)ₙNH₂, —COOR^{a}, —CONHR^{b}, —CONH(CH₂)ₙCOOR^{a}, —NHCOOR^{a}, —CF₃, CHF₂, or CH₂F;

R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently 1, 2, 3, 4, 5 or 6; and

n is independently 0, 1, 2, 3, 4, 5, 6, 7, 8, 9, or 10.

Another aspect of the invention relates to compounds of Formula IX:

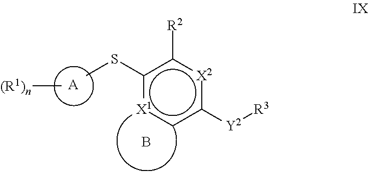

and pharmaceutically acceptable salts, prodrugs, solvates, hydrates, tautomers, or isomers thereof, wherein:

A is selected from the group consisting of 5- to 12-membered monocyclic or polycyclic cycloalkyl, heterocycloalkyl, aryl, or heteroaryl;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

X¹ is N or C;

X² is N or CH;

B, including the atoms at the points of attachment, is a monocyclic or polycyclic 5- to 12-membered heterocycle or a monocyclic or polycyclic 5- to 12-membered heteroaryl;

R² is independently H, —OR^{b}, —NR⁵R⁶, —CN, —C₁-C₆alkyl, —C₂-C₆alkenyl, —C₄-C₈cycloalkenyl, —C₂-C₆alkynyl, —NH₂, halogen, —C(O)OR^{a}, —C₃-C₈cycloalkyl, aryl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, aryl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the ring and the bond on the right side of the Y² moiety, as drawn, is bound to R³;

R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently —H, -D, —C₁-C₆alkyl, —C₁-C₆cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —R⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆alkyl, CF₃, CHF₂, or CH₂F;

R³ is independently, at each occurrence, selected from the group consisting of —H, —C₁-C₆alkyl, a 3- to 12-membered monocyclic or polycyclic heterocycle, C₃-C₈cycloalkyl, or —(CH₂)ₙ—R, wherein each alkyl, heterocycle, or cycloalkyl is optionally substituted with one or more —C₁-C₆alkyl, —OH, —NH₂, —OR^{a}, —NHR^{a}, —(CH₂)ₙOH, heterocyclyl, or spiroheterocyclyl; or

R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, —NH₂, heteroaryl, heterocyclyl, —(CH₂)ₙNH₂, —COOR^{a}, —CONHR^{b}, —CONH(CH₂)ₙCOOR^{a}, —NHCOOR^{a}, —CF₃, CHF₂, or CH₂F;

R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently 1, 2, 3, 4, 5 or 6; and

n is independently 0, 1, 2, 3, 4, 5, 6, 7, 8, 9, or 10.

Another aspect of the invention relates to compounds of Formula X:

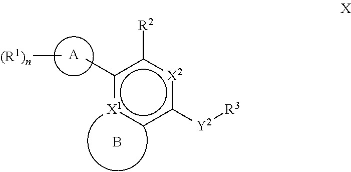

and pharmaceutically acceptable salts, prodrugs, solvates, hydrates, tautomers, or isomers thereof, wherein:

A is selected from the group consisting of 5- to 12-membered monocyclic or polycyclic cycloalkyl, heterocycloalkyl, aryl, or heteroaryl;

R¹ is independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, —OH, halogen, —NO₂, —CN, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, —C(O)R⁵, or —CO₂R⁵, wherein each alkyl, alkenyl, cycloalkenyl, alkynyl, or cycloalkyl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl;

X¹ is N or C;

X² is N or CH;

B, including the atoms at the points of attachment, is a monocyclic or polycyclic 5- to 12-membered heterocycle or a monocyclic or polycyclic 5- to 12-membered heteroaryl;

R² is independently H, —OR^{b}, —NR⁵R⁶, —CN, —C₁-C₆alkyl, —C₂-C₆alkenyl, —C₄-C₈cycloalkenyl, —C₂-C₆alkynyl, —NH₂, halogen, —C(O)OR^{a}, —C₃-C₈cycloalkyl, heterocyclyl containing 1-5 heteroatoms selected from the group consisting of N, S, P, or O, or heteroaryl containing 1-5 heteroatoms selected from the group consisting of N, S, P, or O; wherein each alkyl, alkenyl, cycloalkenyl, alkynyl, cycloalkyl, heterocyclyl, or heteroaryl is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, or heteroaryl; and wherein the heterocyclyl or heteroaryl is not attached via a nitrogen atom;

Y² is selected from the group consisting of: —NR^{a}—, —(CR^{a}₂)ₘ—, —C(O)—, —C(R^{a})₂NH—, —(CR^{a}₂)ₘO—, —C(O)N(R^{a})—, —N(R^{a})C(O)—, —S(O)₂N(R^{a})—, —N(R^{a})S(O)₂—, —N(R^{a})C(O)N(R^{a})—, —N(R^{a})C(S)N(R^{a})—, —C(O)O—, —OC(O)—, —OC(O)N(R^{a})—, —N(R^{a})C(O)O—, —C(O)N(R^{a})O—, —N(R^{a})C(S)—, —C(S)N(R^{a})—, and —OC(O)O—; wherein the bond on the left side of Y², as drawn, is bound to the ring and the bond on the right side of the Y² moiety, as drawn, is bound to R³;

R^{a} is independently, at each occurrence, selected from the group consisting of —H, -D, —OH, —C₃-C₈cycloalkyl, and —C₁-C₆alkyl, wherein each alkyl or cycloalkyl is optionally substituted with one or more —NH₂, wherein 2 R^{a}, together with the carbon atom to which they are both attached, can combine to form a 3- to 8-membered cycloalkyl;

R^{b} is independently —H, -D, —C₁-C₆alkyl, —C₁-C₆cycloalkyl, —C₂-C₆alkenyl, or heterocyclyl containing 1-5 heteroatoms selected from the group consisting of N, S, P, or O; wherein each alkyl, cycloalkyl, alkenyl, or heterocycle is optionally substituted with one or more —OH, halogen, —NO₂, oxo, —CN, —R⁵, —OR⁵, —NR⁵R⁶, —SR⁵, —S(O)₂NR⁵R⁶, —S(O)₂R⁵, —NR⁵S(O)₂NR⁵R⁶, —NR⁵S(O)₂R⁶, —S(O)NR⁵R⁶, —S(O)R⁵, —NR⁵S(O)NR⁵R⁶, —NR⁵S(O)R⁶, heterocycle, aryl, heteroaryl, —(CH₂)ₙOH, —C₁-C₆alkyl, CF₃, CHF₂, or CH₂F;

R³ is independently, at each occurrence, selected from the group consisting of —H, —C₁-C₆alkyl, a 3- to 12-membered monocyclic or polycyclic heterocycle, C₃-C₈cycloalkyl, or —(CH₂)ₙ—R^{b}, wherein each alkyl, heterocycle, or cycloalkyl is optionally substituted with one or more —C₁-C₆alkyl, —OH, —NH₂, —OR^{a}, —NHR^{a}, —(CH₂)ₙOH, heterocyclyl, or spiroheterocyclyl; or

R³ can combine with R^{a} to form a 3- to 12-membered monocyclic or polycyclic heterocycle, or a 5- to 12-membered spiroheterocycle, wherein each heterocycle or spiroheterocycle is optionally substituted with —C₁-C₆alkyl, —OH, —NH₂, heteroaryl, heterocyclyl, —(CH₂)ₙNH₂, —COOR^{a}, —CONHR^{b}, —CONH(CH₂)ₙCOOR^{a}, —NHCOOR^{a}, —CF₃, CHF₂, or CH₂F;

R⁵ and R⁶ are each independently, at each occurrence, selected from the group consisting of —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, —OR⁷, —SR⁷, halogen, —NR⁷R⁸, —NO₂, and —CN;

R⁷ and R⁸ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more —OH, —SH, —NH₂, —NO₂, or —CN;

m is independently 1, 2, 3, 4, 5 or 6; and

n is independently 0, 1, 2, 3, 4, 5, 6, 7, 8, 9, or 10.

Another aspect of the present disclosure relates to compounds, and pharmaceutically acceptable salts, prodrugs, solvates, hydrates, tautomers, or isomers thereof, in Table A1.

TABLE A1

| Cmpd | |
|---|---|
| # | Structure |
| A-1 | 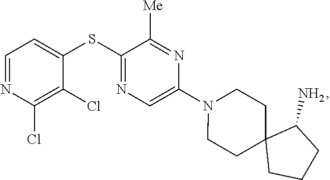 [SMILES: Cc1nc(N2CCC3(CCC[C@H]3N)CC2)cnc1Sc1ccnc(Cl)c1Cl] |
| A-2 | 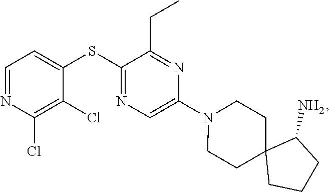 [SMILES: CCc1nc(N2CCC3(CCC[C@H]3N)CC2)cnc1Sc1ccnc(Cl)c1Cl] |
| A-3 | 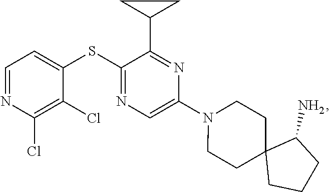 [SMILES: N[C@@H]1CCCC12CCN(c1cnc(Sc3ccnc(Cl)c3Cl)c(C3CC3)n1)CC2] |
| A-4 | 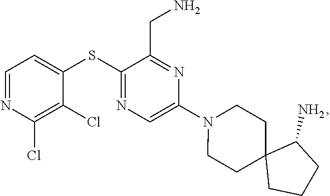 [SMILES: NCc1nc(N2CCC3(CCC[C@H]3N)CC2)cnc1Sc1ccnc(Cl)c1Cl] |
| A-5 | 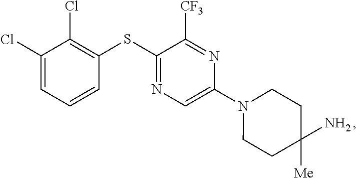 [SMILES: CC1(N)CCN(c2cnc(Sc3cccc(Cl)c3Cl)c(C(F)(F)F)n2)CC1] |
| A-6 | 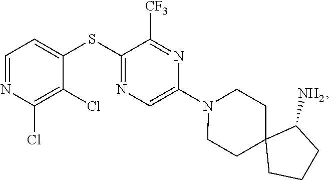 [SMILES: N[C@@H]1CCCC12CCN(c1cnc(Sc3ccnc(Cl)c3Cl)c(C(F)(F)F)n1)CC2] |
| A-7 | 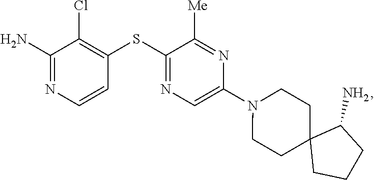 [SMILES: Cc1nc(N2CCC3(CCC[C@H]3N)CC2)cnc1Sc1ccnc(N)c1Cl] |
| A-8 | 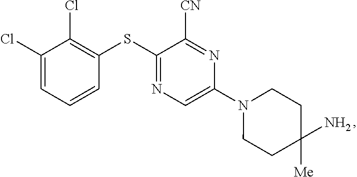 [SMILES: CC1(N)CCN(c2cnc(Sc3cccc(Cl)c3Cl)c(C#N)n2)CC1] |
| A-9 | 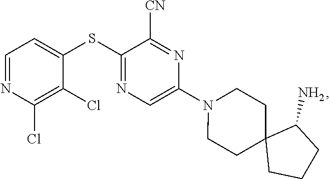 [SMILES: N#Cc1nc(N2CCC3(CCC[C@H]3N)CC2)cnc1Sc1ccnc(Cl)c1Cl] |
| A-10 | 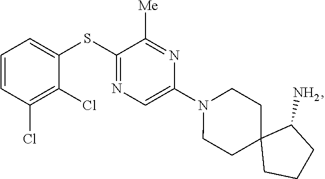 [SMILES: Cc1nc(N2CCC3(CCC[C@H]3N)CC2)cnc1Sc1cccc(Cl)c1Cl] |
| A-11 | 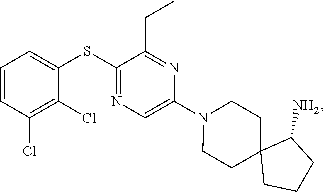 [SMILES: CCc1nc(N2CCC3(CCC[C@H]3N)CC2)cnc1Sc1cccc(Cl)c1Cl] |
| A-12 | 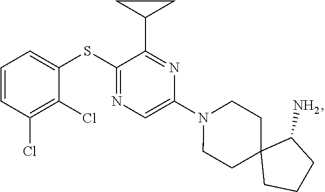 [SMILES: N[C@@H]1CCCC12CCN(c1cnc(Sc3cccc(Cl)c3Cl)c(C3CC3)n1)CC2] |
| A-13 | 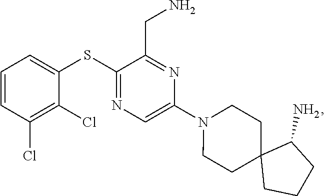 [SMILES: NCc1nc(N2CCC3(CCC[C@H]3N)CC2)cnc1Sc1cccc(Cl)c1Cl] |
| A-14 | 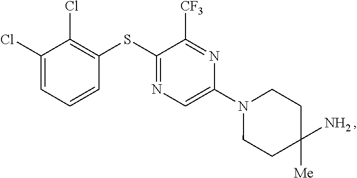 [SMILES: CC1(N)CCN(c2cnc(Sc3cccc(Cl)c3Cl)c(C(F)(F)F)n2)CC1] |
| A-15 | 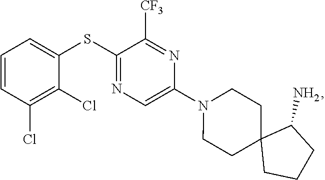 [SMILES: N[C@@H]1CCCC12CCN(c1cnc(Sc3cccc(Cl)c3Cl)c(C(F)(F)F)n1)CC2] |
| A-16 | 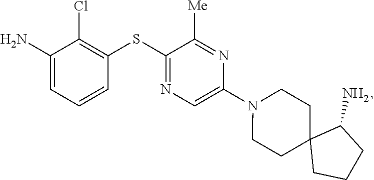 [SMILES: Cc1nc(N2CCC3(CCC[C@H]3N)CC2)cnc1Sc1cccc(N)c1Cl] |
| A-17 | 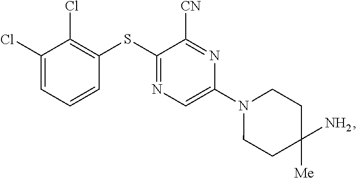 [SMILES: CC1(N)CCN(c2cnc(Sc3cccc(Cl)c3Cl)c(C#N)n2)CC1] |
| A-18 | 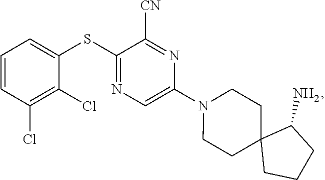 [SMILES: N#Cc1nc(N2CCC3(CCC[C@H]3N)CC2)cnc1Sc1cccc(Cl)c1Cl] |
| A-19 | 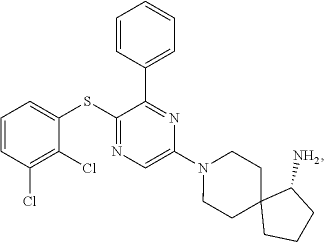 [SMILES: N[C@@H]1CCCC12CCN(c1cnc(Sc3cccc(Cl)c3Cl)c(-c3ccccc3)n1)CC2] |
| A-20 | 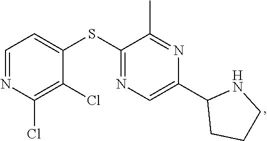 [SMILES: Cc1nc(C2CCCN2)cnc1Sc1ccnc(Cl)c1Cl] |
| A-21 | 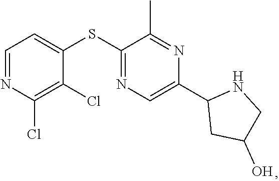 [SMILES: Cc1nc(C2CC(O)CN2)cnc1Sc1ccnc(Cl)c1Cl] |
| A-22 | 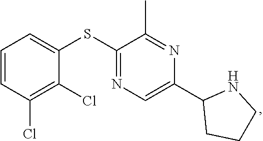 [SMILES: Cc1nc(C2CCCN2)cnc1Sc1cccc(Cl)c1Cl] |
| A-23 | 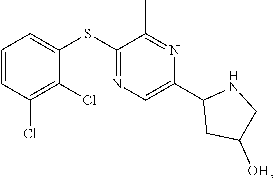 [SMILES: Cc1nc(C2CC(O)CN2)cnc1Sc1cccc(Cl)c1Cl] |
| A-24 | 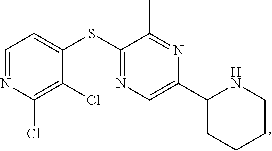 [SMILES: Cc1nc(C2CCCCN2)cnc1Sc1ccnc(Cl)c1Cl] |
| A-25 | 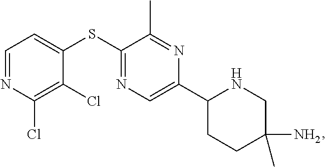 [SMILES: Cc1nc(C2CCC(C)(N)CN2)cnc1Sc1ccnc(Cl)c1Cl] |
| A-26 | 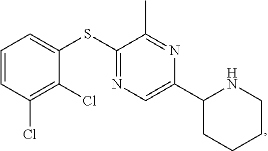 [SMILES: Cc1nc(C2CCCCN2)cnc1Sc1cccc(Cl)c1Cl] |
| A-27 | 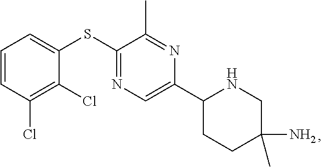 [SMILES: Cc1nc(C2CCC(C)(N)CN2)cnc1Sc1cccc(Cl)c1Cl] |
| A-28 | 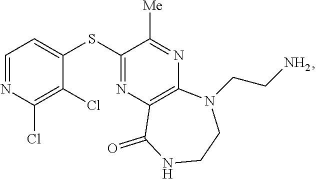 [SMILES: Cc1nc2c(nc1Sc1ccnc(Cl)c1Cl)C(=O)NCCN2CCN] |
| A-29 | 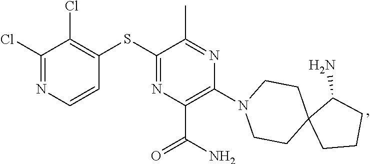 [SMILES: Cc1nc(N2CCC3(CCC[C@H]3N)CC2)c(C(N)=O)nc1Sc1ccnc(Cl)c1Cl] |
| A-30 | 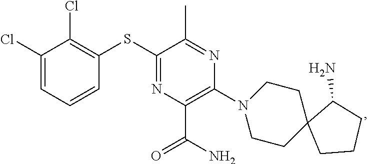 [SMILES: Cc1nc(N2CCC3(CCC[C@H]3N)CC2)c(C(N)=O)nc1Sc1cccc(Cl)c1Cl] |
| A-31 | 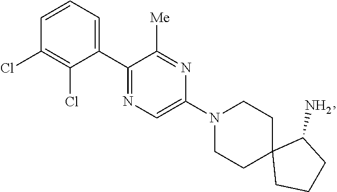 [SMILES: Cc1nc(N2CCC3(CCC[C@H]3N)CC2)cnc1-c1cccc(Cl)c1Cl] |
| A-32 | 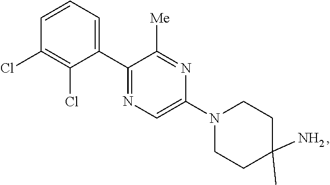 [SMILES: Cc1nc(N2CCC(C)(N)CC2)cnc1-c1cccc(Cl)c1Cl] |
| A-33 | 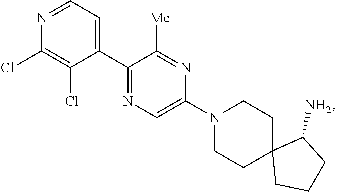 [SMILES: Cc1nc(N2CCC3(CCC[C@H]3N)CC2)cnc1-c1ccnc(Cl)c1Cl] |
| A-34 | 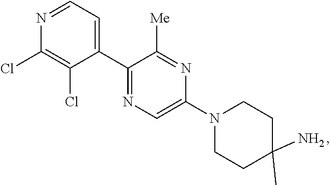 [SMILES: Cc1nc(N2CCC(C)(N)CC2)cnc1-c1ccnc(Cl)c1Cl] |
| A-35 | 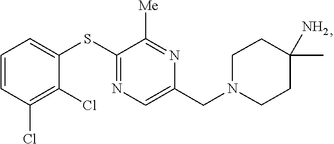 [SMILES: Cc1nc(CN2CCC(C)(N)CC2)cnc1Sc1cccc(Cl)c1Cl] |
| A-36 | 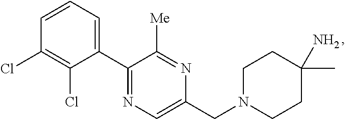 [SMILES: Cc1nc(CN2CCC(C)(N)CC2)cnc1-c1cccc(Cl)c1Cl] |
| A-37 | 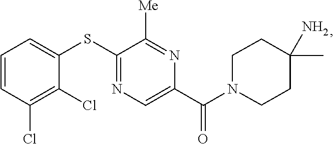 [SMILES: Cc1nc(C(=O)N2CCC(C)(N)CC2)cnc1Sc1cccc(Cl)c1Cl] |
| A-38 | 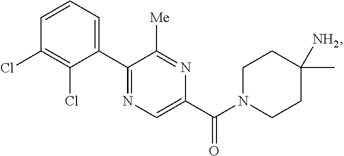 [SMILES: Cc1nc(C(=O)N2CCC(C)(N)CC2)cnc1-c1cccc(Cl)c1Cl] |
| A-39 | 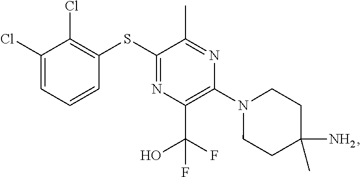 [SMILES: Cc1nc(N2CCC(C)(N)CC2)c(C(O)(F)F)nc1Sc1cccc(Cl)c1Cl] |
| A-40 | 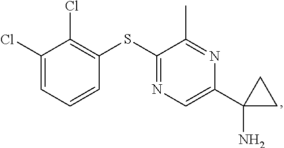 [SMILES: Cc1nc(C2(N)CC2)cnc1Sc1cccc(Cl)c1Cl] |
| A-41 | 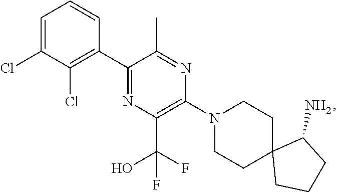 [SMILES: Cc1nc(N2CCC3(CCC[C@H]3N)CC2)c(C(O)(F)F)nc1-c1cccc(Cl)c1Cl] |
| B-1 | 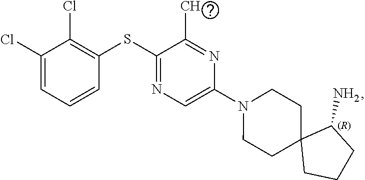 |
| B-2 | 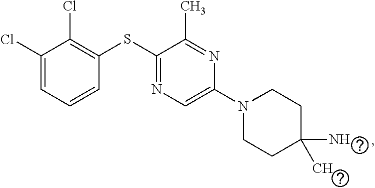 |
| B-3 | 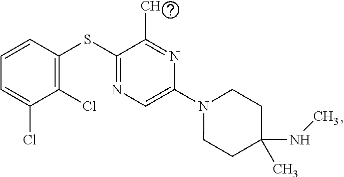 [SMILES: CNC1(C)CCN(c2cnc(Sc3cccc(Cl)c3Cl)c(C)n2)CC1] |
| B-4 | 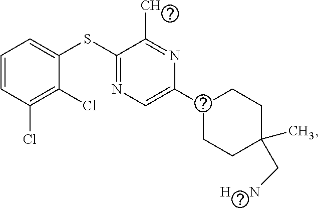 |
| B-5 | 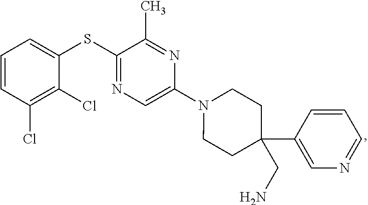 [SMILES: Cc1nc(N2CCC(CN)(c3cccnc3)CC2)cnc1Sc1cccc(Cl)c1Cl] |
| B-6 | 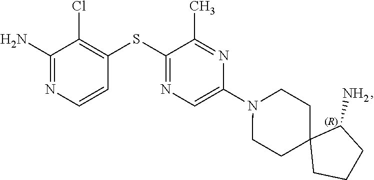 |
| B-7 | 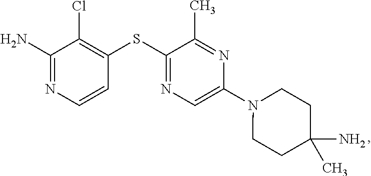 [SMILES: Cc1nc(N2CCC(C)(N)CC2)cnc1Sc1ccnc(N)c1Cl] |
| B-8 | 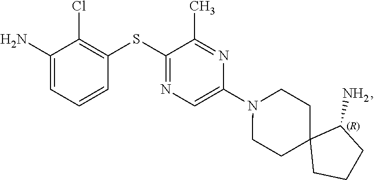 |
| B-9 | 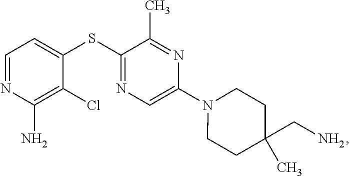 [SMILES: Cc1nc(N2CCC(C)(CN)CC2)cnc1Sc1ccnc(N)c1Cl] |
| B-10 | 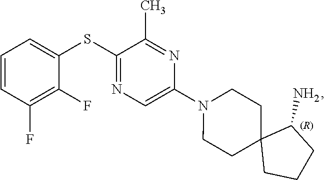 |
| B-11 | 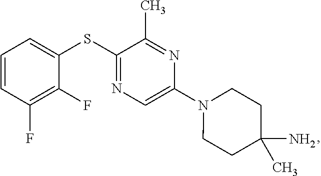 [SMILES: Cc1nc(N2CCC(C)(N)CC2)cnc1Sc1cccc(F)c1F] |
| B-12 | 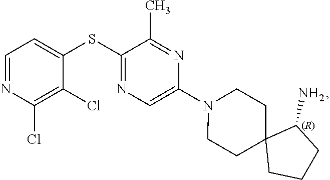 |
| B-13 | 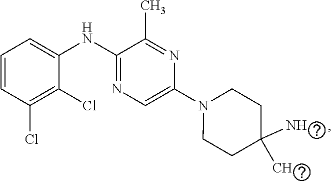 [SMILES: Cc1nc(N2CCC(C)(N)CC2)cnc1Nc1cccc(Cl)c1Cl] |
| B-14 | 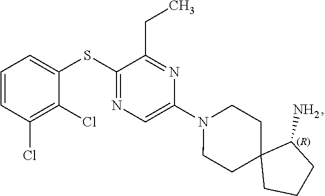 |
| B-15 | 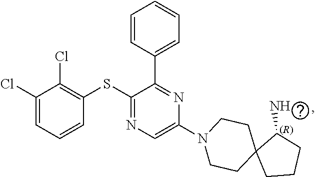 |
| B-16 | 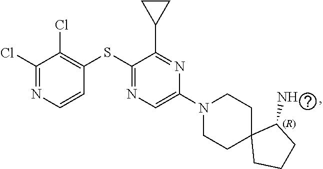 |
| B-17 | 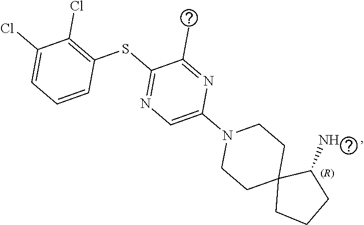 |
| B-18 | 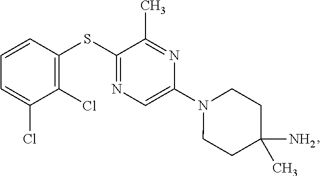 [SMILES: Cc1nc(N2CCC(C)(N)CC2)cnc1Sc1cccc(Cl)c1Cl] |
| B-19 | 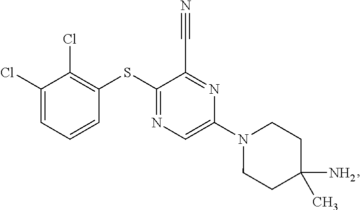 [SMILES: CC1(N)CCN(c2cnc(Sc3cccc(Cl)c3Cl)c(C#N)n2)CC1] |
| B-20 | 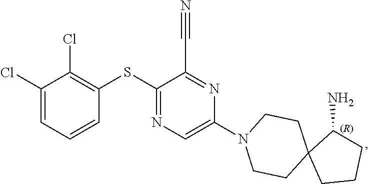 |
| B-21 | 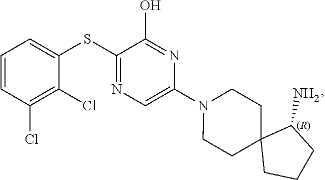 |
| B-22 | 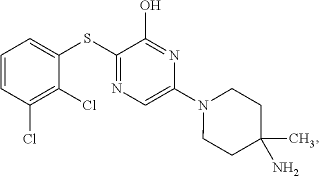 [SMILES: CC1(N)CCN(c2cnc(Sc3cccc(Cl)c3Cl)c(O)n2)CC1] |
| B-23 | 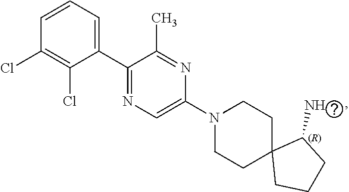 |
| B-24 | 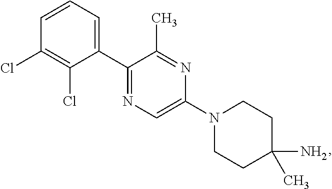 [SMILES: Cc1nc(N2CCC(C)(N)CC2)cnc1-c1cccc(Cl)c1Cl] |
| B-25 | 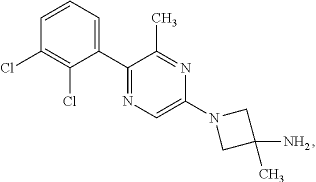 [SMILES: Cc1nc(N2CC(C)(N)C2)cnc1-c1cccc(Cl)c1Cl] |
| B-26 | 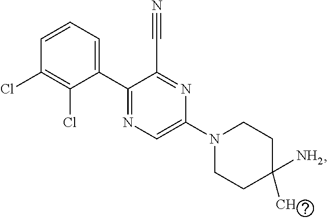 [SMILES: CC1(N)CCN(c2cnc(-c3cccc(Cl)c3Cl)c(C#N)n2)CC1] |
| B-27 | 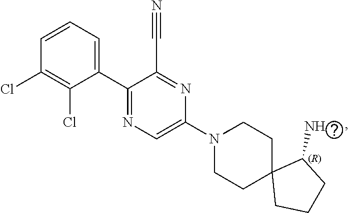 |
| B-28 | 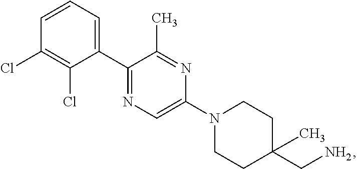 [SMILES: Cc1nc(N2CCC(C)(CN)CC2)cnc1-c1cccc(Cl)c1Cl] |
| B-29 | 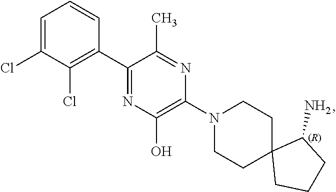 |
| B-30 | 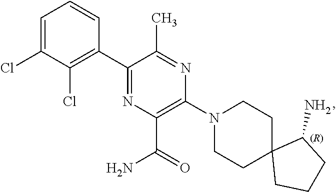 |
| B-31 | 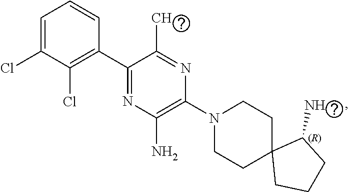 |
| B-32 | 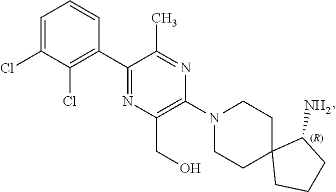 |
| B-33 | 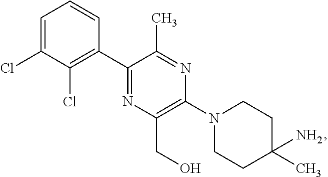 [SMILES: Cc1nc(N2CCC(C)(N)CC2)c(CO)nc1-c1cccc(Cl)c1Cl] |
| B-34 | 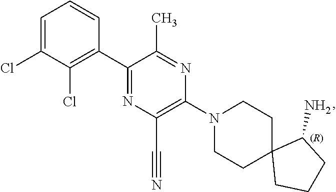 |
| B-35 | 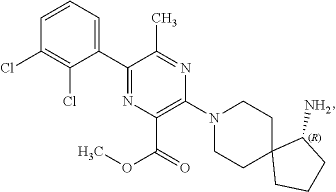 |
| B-36 | 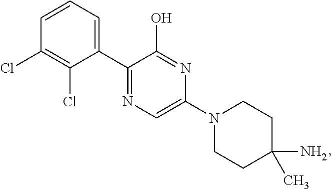 [SMILES: CC1(N)CCN(c2cnc(-c3cccc(Cl)c3Cl)c(O)n2)CC1] |
| B-37 | 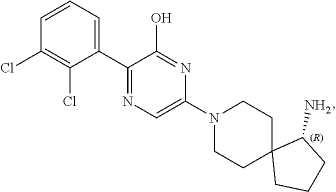 |
| B-38 | 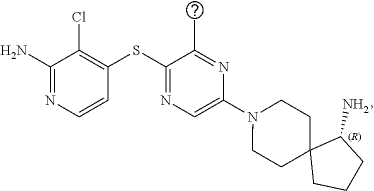 |
| B-39 | 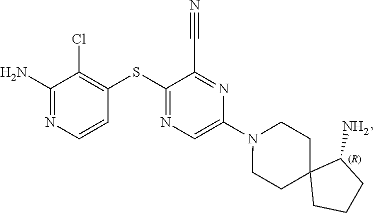 |
| B-40 | 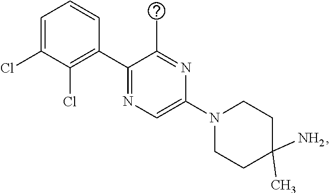 [SMILES: Cc1nc(N2CCC(C)(N)CC2)cnc1-c1cccc(Cl)c1Cl] |
| B-41 | 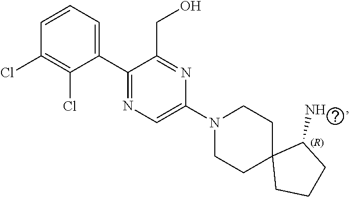 |
| B-42 | 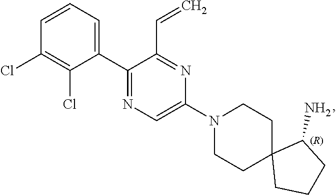 |
| B-43 | 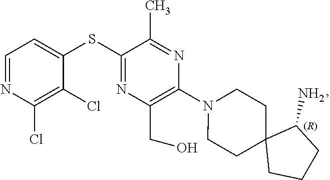 |
| B-44 | 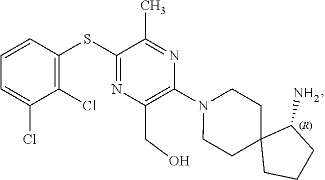 |
| B-45 | 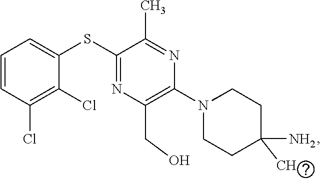 |
| B-46 | 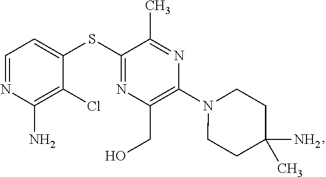 [SMILES: Cc1nc(N2CCC(C)(N)CC2)c(CO)nc1Sc1ccnc(N)c1Cl] |
| B-47 | 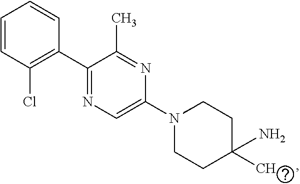 |
| B-48 | 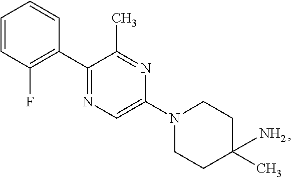 [SMILES: Cc1nc(N2CCC(C)(N)CC2)cnc1-c1ccccc1F] |
| B-49 | 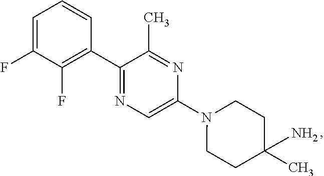 [SMILES: Cc1nc(N2CCC(C)(N)CC2)cnc1-c1cccc(F)c1F] |
| B-50 | 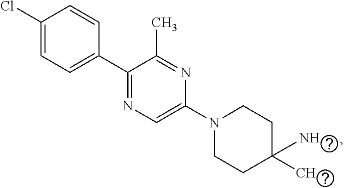 [SMILES: Cc1nc(N2CCC(C)(N)CC2)cnc1-c1ccc(Cl)cc1] |
| B-51 | 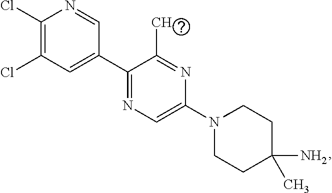 |
| B-52 | 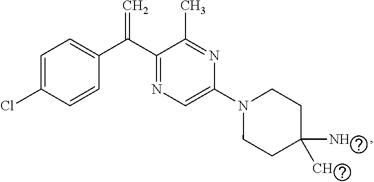 [SMILES: C=C(c1ccc(Cl)cc1)c1ncc(N2CCC(C)(N)CC2)nc1C] |
| B-53 | 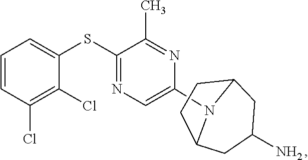 [SMILES: Cc1nc(N2C3CCC2CC(N)C3)cnc1Sc1cccc(Cl)c1Cl] |
| B-54 | 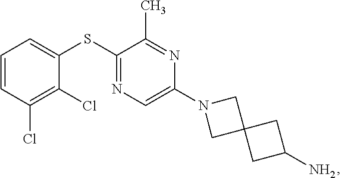 [SMILES: Cc1nc(N2CC3(CC(N)C3)C2)cnc1Sc1cccc(Cl)c1Cl] |
| B-55 | 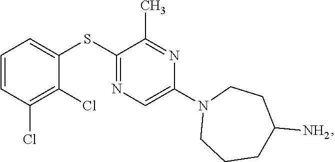 [SMILES: Cc1nc(N2CCCC(N)CC2)cnc1Sc1cccc(Cl)c1Cl] |
| B-56 | 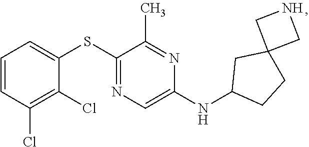 [SMILES: Cc1nc(NC2CCC3(CNC3)C2)cnc1Sc1cccc(Cl)c1Cl] |
| B-57 | 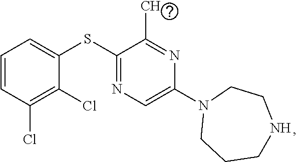 [SMILES: Cc1nc(N2CCCNCC2)cnc1Sc1cccc(Cl)c1Cl] |
| B-58 | 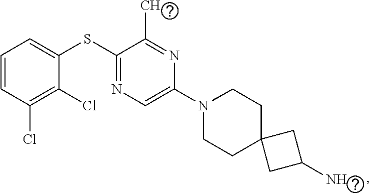 |
| B-59 | 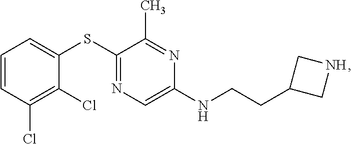 [SMILES: Cc1nc(NCCC2CNC2)cnc1Sc1cccc(Cl)c1Cl] |
| B-60 | 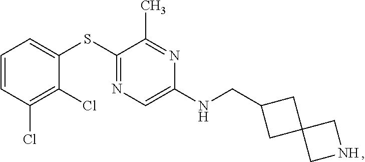 [SMILES: Cc1nc(NCC2CC3(CNC3)C2)cnc1Sc1cccc(Cl)c1Cl] |
| B-61 | 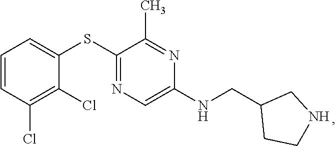 [SMILES: Cc1nc(NCC2CCNC2)cnc1Sc1cccc(Cl)c1Cl] |
| B-62 | 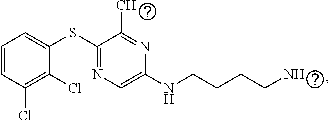 [SMILES: Cc1nc(NCCCCN)cnc1Sc1cccc(Cl)c1Cl] |
| B-63 | 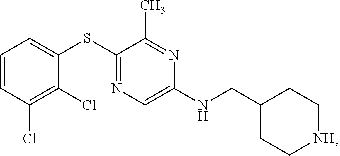 [SMILES: Cc1nc(NCC2CCNCC2)cnc1Sc1cccc(Cl)c1Cl] |
| B-64 | 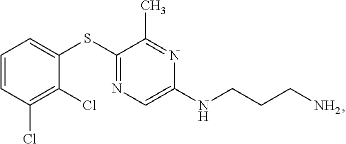 [SMILES: Cc1nc(NCCCN)cnc1Sc1cccc(Cl)c1Cl] |
| B-65 | 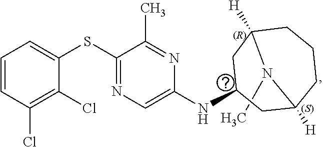 |
| B-66 | 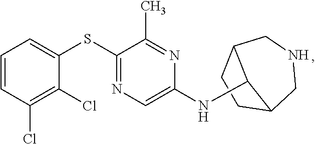 [SMILES: Cc1nc(NC2C3CCC2CNC3)cnc1Sc1cccc(Cl)c1Cl] |
| B-67 | 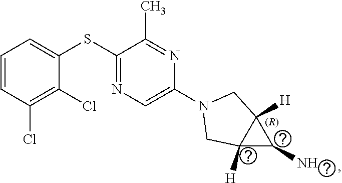 |
| B-68 | 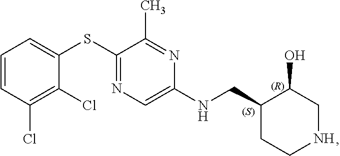 |
| B-69 | 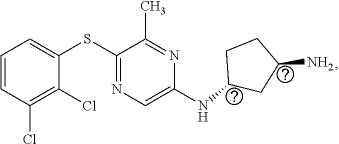 [SMILES: Cc1nc(N[C@@H]2CC[C@@H](N)C2)cnc1Sc1cccc(Cl)c1Cl] |
| B-70 | 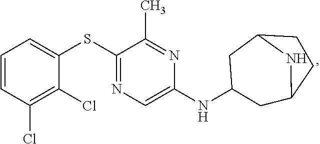 [SMILES: Cc1nc(NC2CC3CCC(C2)N3)cnc1Sc1cccc(Cl)c1Cl] |
| B-71 | 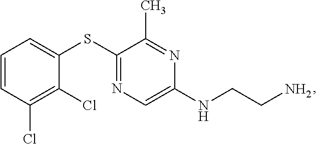 [SMILES: Cc1nc(NCCN)cnc1Sc1cccc(Cl)c1Cl] |
| B-72 | 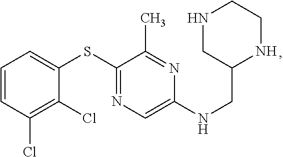 [SMILES: Cc1nc(NCC2CNCCN2)cnc1Sc1cccc(Cl)c1Cl] |
| B-73 | 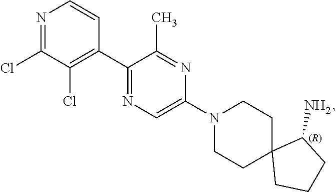 |
| B-74 | 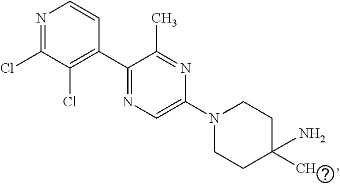 |
| B-75 | 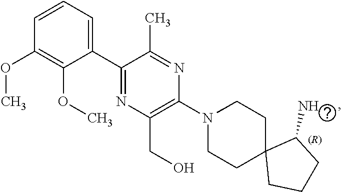 |
| B-76 | 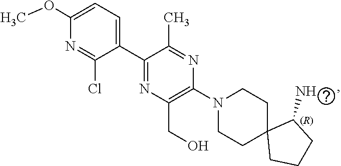 |
| B-77 | 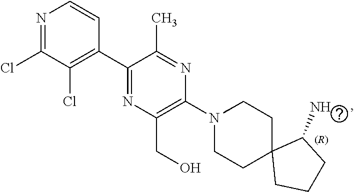 |
| B-78 | 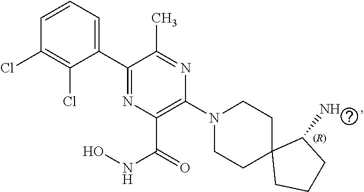 |
| B-79 | 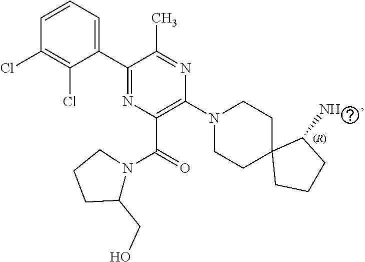 |
| B-80 | 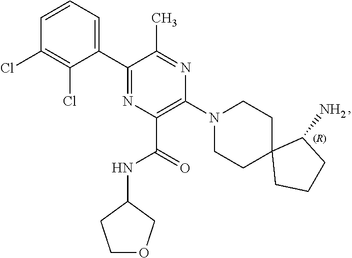 |
| B-81 | 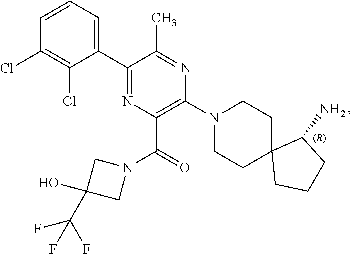 |
| B-82 | 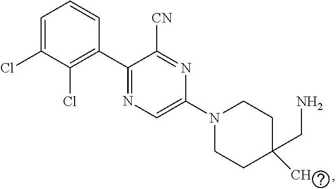 [SMILES: CC1(CN)CCN(c2cnc(-c3cccc(Cl)c3Cl)c(C#N)n2)CC1] |
| B-83 | 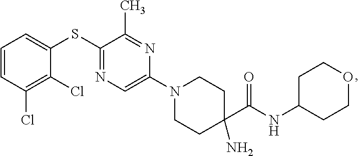 [SMILES: Cc1nc(N2CCC(N)(C(=O)NC3CCOCC3)CC2)cnc1Sc1cccc(Cl)c1Cl] |
| B-84 | 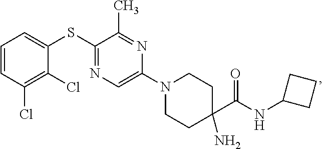 [SMILES: Cc1nc(N2CCC(N)(C(=O)NC3CCC3)CC2)cnc1Sc1cccc(Cl)c1Cl] |
| B-85 | 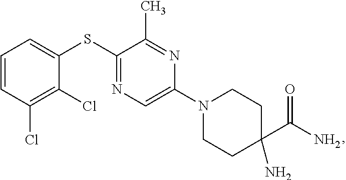 [SMILES: Cc1nc(N2CCC(N)(C(N)=O)CC2)cnc1Sc1cccc(Cl)c1Cl] |
| B-86 | 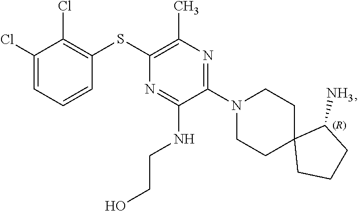 |
| B-87 | 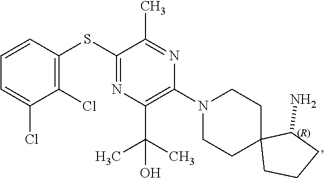 |
| B-88 | 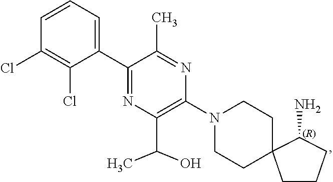 |
| B-89 | 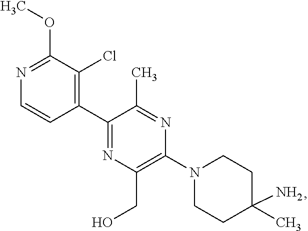 [SMILES: COc1nccc(-c2nc(CO)c(N3CCC(C)(N)CC3)nc2C)c1Cl] |
| B-90 | 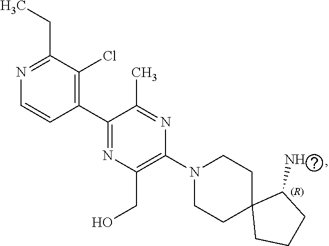 |
| B-91 | 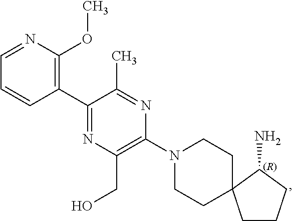 |
| B-92 | 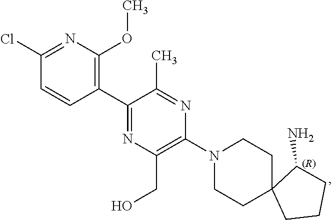 |
| B-93 | 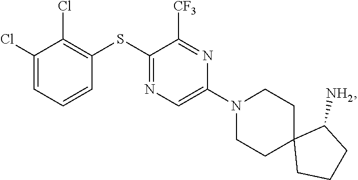 [SMILES: N[C@@H]1CCCC12CCN(c1cnc(Sc3cccc(Cl)c3Cl)c(C(F)(F)F)n1)CC2] |
| B-94 | 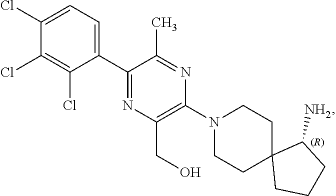 |
| B-95 | 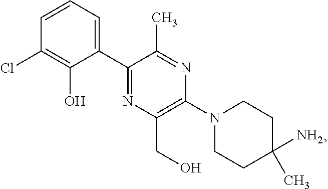 [SMILES: Cc1nc(N2CCC(C)(N)CC2)c(CO)nc1-c1cccc(Cl)c1O] |
| B-96 | 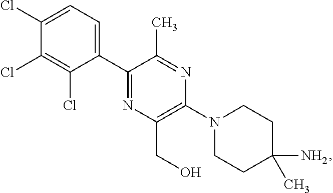 [SMILES: Cc1nc(N2CCC(C)(N)CC2)c(CO)nc1-c1ccc(Cl)c(Cl)c1Cl] |
| B-97 | 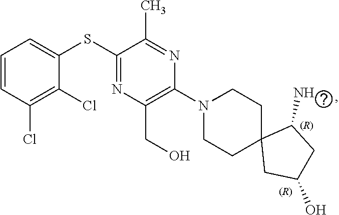 |
| B-98 | 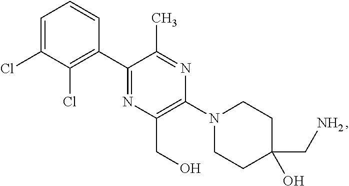 [SMILES: Cc1nc(N2CCC(O)(CN)CC2)c(CO)nc1-c1cccc(Cl)c1Cl] |
| B-99 | 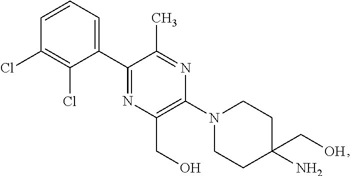 [SMILES: Cc1nc(N2CCC(N)(CO)CC2)c(CO)nc1-c1cccc(Cl)c1Cl] |
| B-100 | 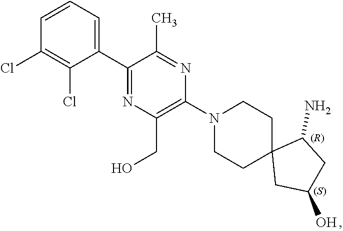 |
| B-101 | 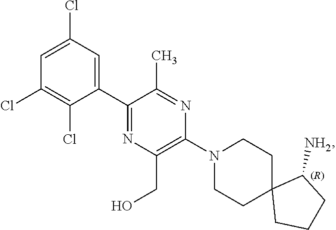 |
| B-102 | 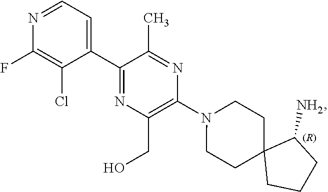 |
| B-103 | 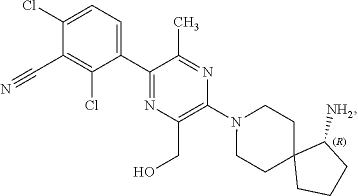 |
| B-104 | 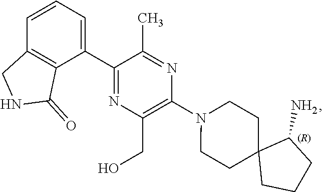 |
| B-105 | 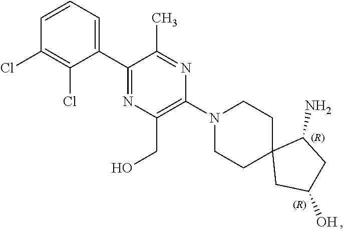 |
| B-106 | 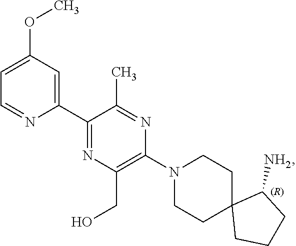 |
| B-107 | 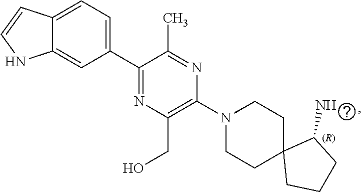 |
| B-108 | 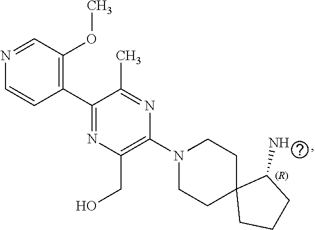 |
| B-109 | 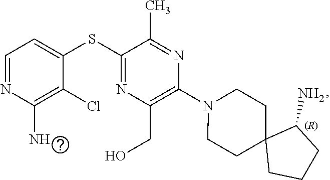 |
| B-110 | 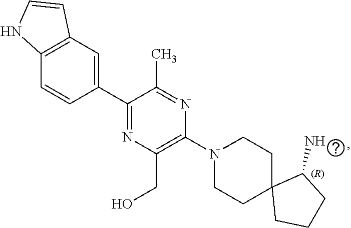 |
| B-111 | 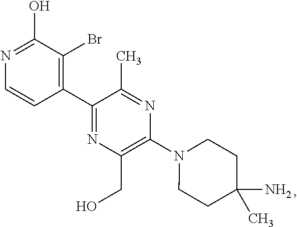 [SMILES: Cc1nc(N2CCC(C)(N)CC2)c(CO)nc1-c1ccnc(O)c1Br] |
| B-112 | 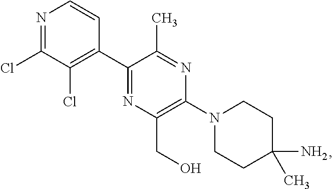 [SMILES: Cc1nc(N2CCC(C)(N)CC2)c(CO)nc1-c1ccnc(Cl)c1Cl] |
| B-113 | 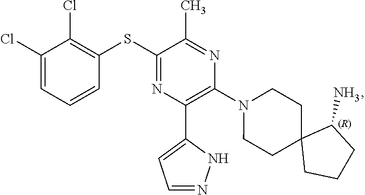 |
| B-114 | 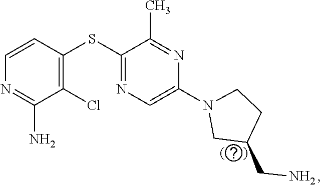 |
| B-115 | 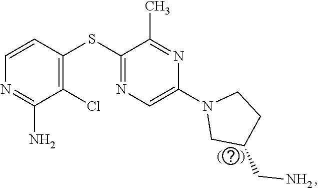 |
| B-116 | 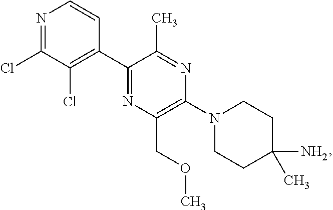 [SMILES: COCc1nc(-c2ccnc(Cl)c2Cl)c(C)nc1N1CCC(C)(N)CC1] |
| B-117 | 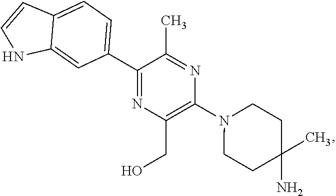 [SMILES: Cc1nc(N2CCC(C)(N)CC2)c(CO)nc1-c1ccc2cc[nH]c2c1] |
| B-118 | 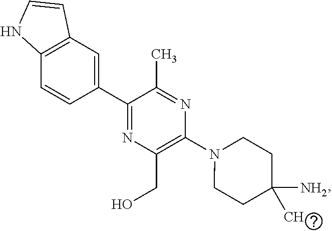 [SMILES: Cc1nc(N2CCC(C)(N)CC2)c(CO)nc1-c1ccc2[nH]ccc2c1] |
| B-119 | 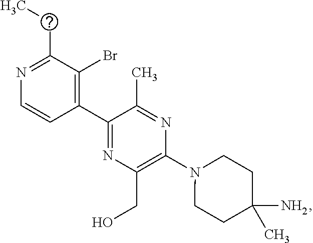 [SMILES: CCc1nccc(-c2nc(CO)c(N3CCC(C)(N)CC3)nc2C)c1Br] |
| B-120 | 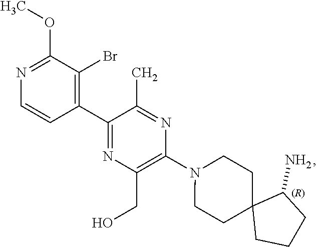 |
| B-121 | 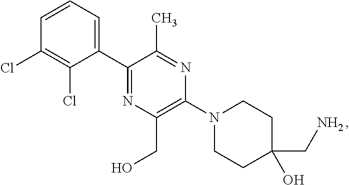 [SMILES: Cc1nc(N2CCC(O)(CN)CC2)c(CO)nc1-c1cccc(Cl)c1Cl] |
| B-122 | 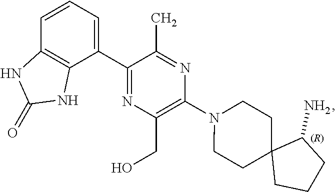 |
| B-123 | 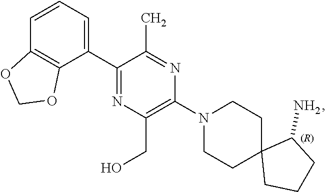 |
| B-124 | 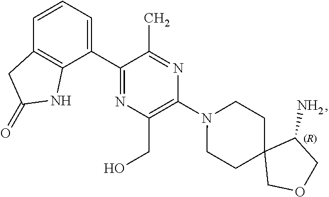 |
| B-125 | 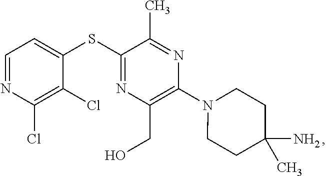 [SMILES: Cc1nc(N2CCC(C)(N)CC2)c(CO)nc1Sc1ccnc(Cl)c1Cl] |
| B-126 | 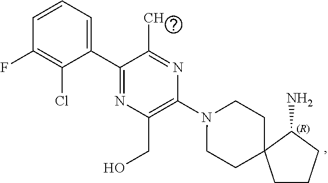 |
| B-127 | 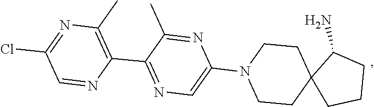 [SMILES: Cc1nc(Cl)cnc1-c1ncc(N2CCC3(CCC[C@H]3N)CC2)nc1C] |
| B-128 | 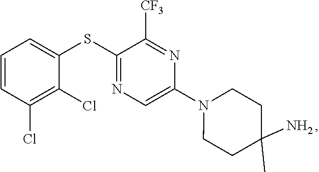 [SMILES: CC1(N)CCN(c2cnc(Sc3cccc(Cl)c3Cl)c(C(F)(F)F)n2)CC1] |
| B-129 | 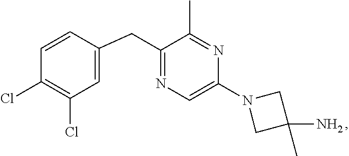 [SMILES: Cc1nc(N2CC(C)(N)C2)cnc1Cc1ccc(Cl)c(Cl)c1] |
| B-130 | 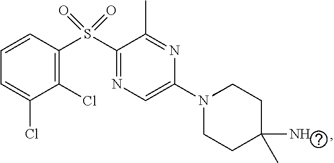 |
| B-131 | 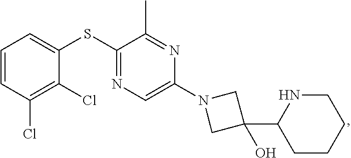 [SMILES: Cc1nc(N2CC(O)(C3CCCCN3)C2)cnc1Sc1cccc(Cl)c1Cl] |
| B-132 | 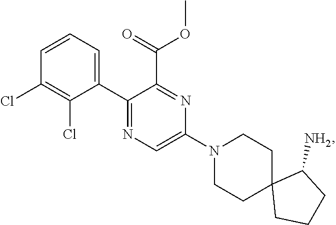 [SMILES: COC(=O)c1nc(N2CCC3(CCC[C@H]3N)CC2)cnc1-c1cccc(Cl)c1Cl] |
| B-133 | 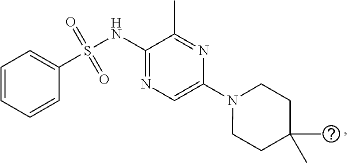 [SMILES: Cc1nc(N2CCC(C)(C)CC2)cnc1NS(=O)(=O)c1ccccc1] |
| B-134 | 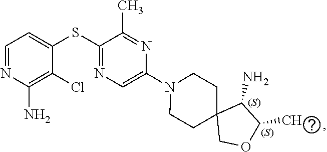 |
| B-135 | 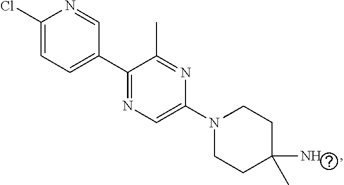 |
| B-136 | 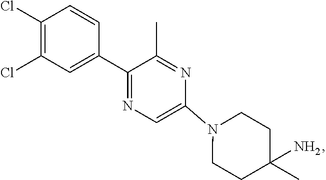 [SMILES: Cc1nc(N2CCC(C)(N)CC2)cnc1-c1ccc(Cl)c(Cl)c1] |
| B-137 | 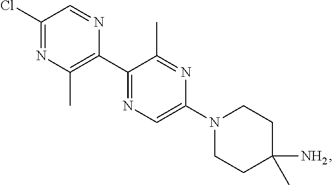 [SMILES: Cc1nc(Cl)cnc1-c1ncc(N2CCC(C)(N)CC2)nc1C] |
| B-138 | 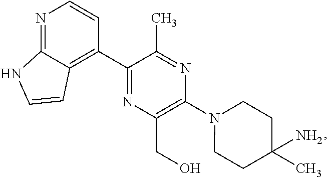 [SMILES: Cc1nc(N2CCC(C)(N)CC2)c(CO)nc1-c1ccnc2[nH]ccc12] |
| B-139 | 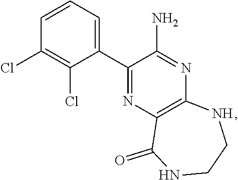 [SMILES: Nc1nc2c(nc1-c1cccc(Cl)c1Cl)C(=O)NCCN2] |
| B-140 | 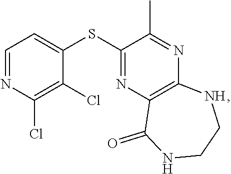 [SMILES: Cc1nc2c(nc1Sc1ccnc(Cl)c1Cl)C(=O)NCCN2] |
| B-141 | 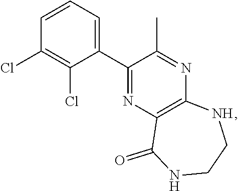 [SMILES: Cc1nc2c(nc1-c1cccc(Cl)c1Cl)C(=O)NCCN2] |
| B-142 | 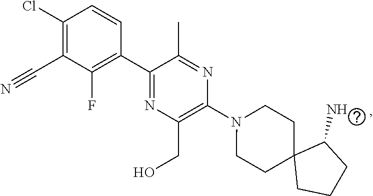 [SMILES: Cc1nc(N2CCC3(CCC[C@H]3N)CC2)c(CO)nc1-c1ccc(Cl)c(C#N)c1F] |
| B-143 | 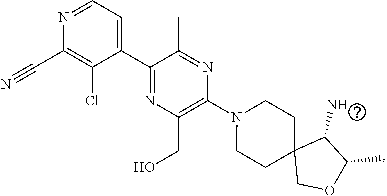 [SMILES: Cc1nc(N2CCC3(CC2)CO[C@@H](C)[C@H]3N)c(CO)nc1-c1ccnc(C#N)c1Cl] |
| B-144 | 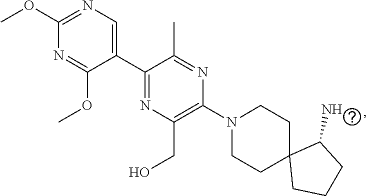 [SMILES: COc1ncc(-c2nc(CO)c(N3CCC4(CCC[C@H]4N)CC3)nc2C)c(OC)n1] |
| B-145 | 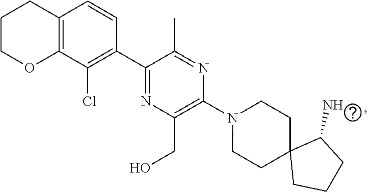 [SMILES: Cc1nc(N2CCC3(CCC[C@H]3N)CC2)c(CO)nc1-c1ccc2c(c1Cl)OCCC2] |
| B-146 | 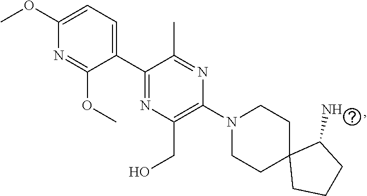 [SMILES: COc1ccc(-c2nc(CO)c(N3CCC4(CCC[C@H]4N)CC3)nc2C)c(OC)n1] |
| B-147 | 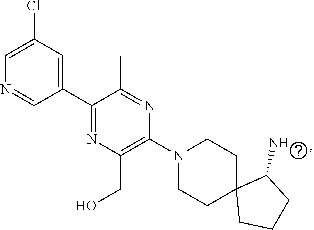 [SMILES: Cc1nc(N2CCC3(CCC[C@H]3N)CC2)c(CO)nc1-c1cncc(Cl)c1] |
| B-148 | 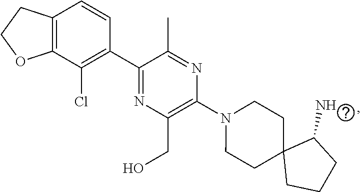 [SMILES: Cc1nc(N2CCC3(CCC[C@H]3N)CC2)c(CO)nc1-c1ccc2c(c1Cl)OCC2] |
| B-149 | 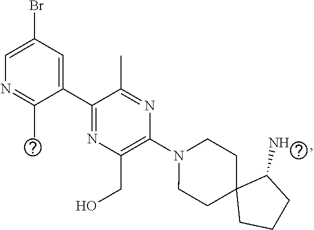 [SMILES: Cc1ncc(Br)cc1-c1nc(CO)c(N2CCC3(CCC[C@H]3N)CC2)nc1C] |
| B-150 | 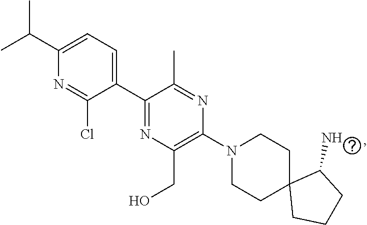 [SMILES: Cc1nc(N2CCC3(CCC[C@H]3N)CC2)c(CO)nc1-c1ccc(C(C)C)nc1Cl] |
| B-151 | 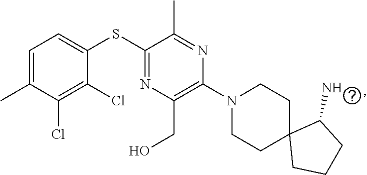 [SMILES: Cc1ccc(Sc2nc(CO)c(N3CCC4(CCC[C@H]4N)CC3)nc2C)c(Cl)c1Cl] |
| B-152 | 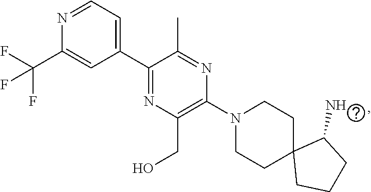 [SMILES: Cc1nc(N2CCC3(CCC[C@H]3N)CC2)c(CO)nc1-c1ccnc(C(F)(F)F)c1] |
| B-153 | 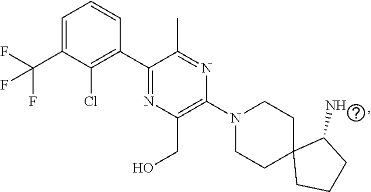 [SMILES: Cc1nc(N2CCC3(CCC[C@H]3N)CC2)c(CO)nc1-c1cccc(C(F)(F)F)c1Cl] |
| B-154 | 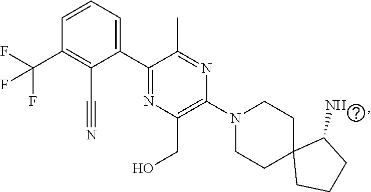 [SMILES: Cc1nc(N2CCC3(CCC[C@H]3N)CC2)c(CO)nc1-c1cccc(C(F)(F)F)c1C#N] |
| B-155 | 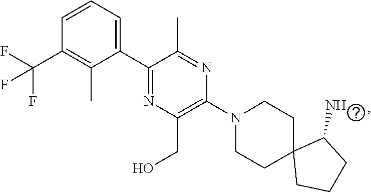 [SMILES: Cc1nc(N2CCC3(CCC[C@H]3N)CC2)c(CO)nc1-c1cccc(C(F)(F)F)c1C] |
| B-156 | 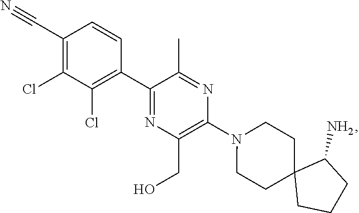 [SMILES: Cc1nc(N2CCC3(CCC[C@H]3N)CC2)c(CO)nc1-c1ccc(C#N)c(Cl)c1Cl] |
| B-157 | 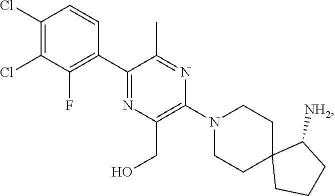 [SMILES: Cc1nc(N2CCC3(CCC[C@H]3N)CC2)c(CO)nc1-c1ccc(Cl)c(Cl)c1F] |
| B-158 | 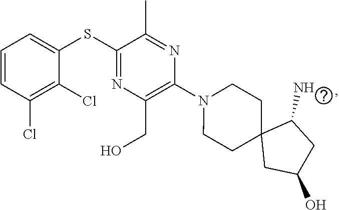 [SMILES: Cc1nc(N2CCC3(CC2)C[C@H](O)C[C@H]3N)c(CO)nc1Sc1cccc(Cl)c1Cl] |
| B-159 | 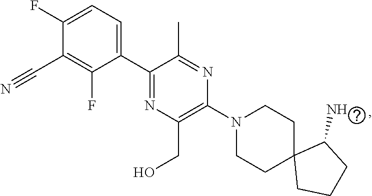 [SMILES: Cc1nc(N2CCC3(CCC[C@H]3N)CC2)c(CO)nc1-c1ccc(F)c(C#N)c1F] |
| B-160 | 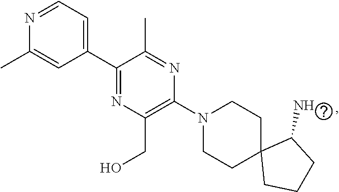 [SMILES: Cc1cc(-c2nc(CO)c(N3CCC4(CCC[C@H]4N)CC3)nc2C)ccn1] |
| B-161 | 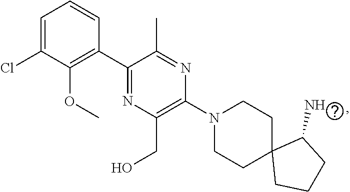 [SMILES: COc1c(Cl)cccc1-c1nc(CO)c(N2CCC3(CCC[C@H]3N)CC2)nc1C] |
| B-162 | 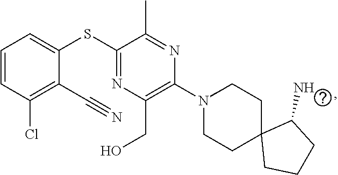 |
| B-163 | 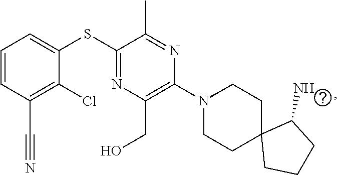 |
| B-164 | 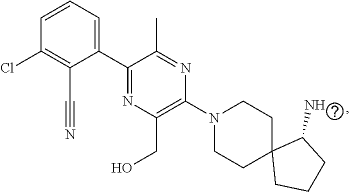 [SMILES: Cc1nc(N2CCC3(CCC[C@H]3N)CC2)c(CO)nc1-c1cccc(Cl)c1C#N] |
| B-165 | 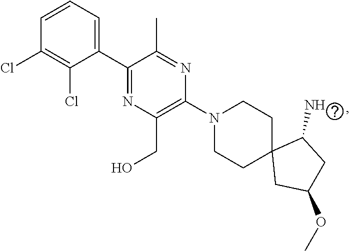 [SMILES: CO[C@@H]1C[C@@H](N)C2(CCN(c3nc(C)c(-c4cccc(Cl)c4Cl)nc3CO)CC2)C1] |
| B-166 | 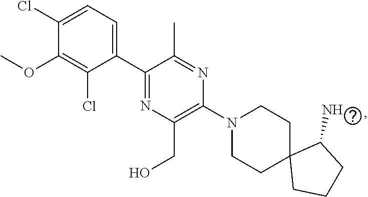 [SMILES: COc1c(Cl)ccc(-c2nc(CO)c(N3CCC4(CCC[C@H]4N)CC3)nc2C)c1Cl] |
| B-167 | 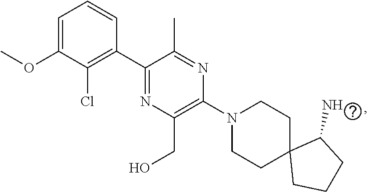 [SMILES: COc1cccc(-c2nc(CO)c(N3CCC4(CCC[C@H]4N)CC3)nc2C)c1Cl] |
| B-168 | 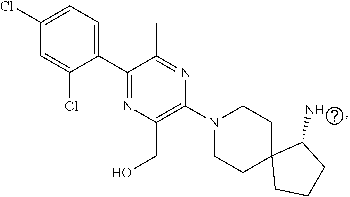 [SMILES: Cc1nc(N2CCC3(CCC[C@H]3N)CC2)c(CO)nc1-c1ccc(Cl)cc1Cl] |
| B-169 | 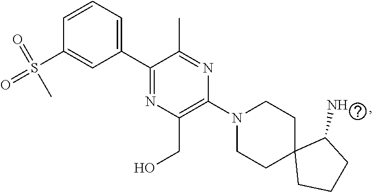 [SMILES: Cc1nc(N2CCC3(CCC[C@H]3N)CC2)c(CO)nc1-c1cccc(S(C)(=O)=O)c1] |
| B-170 | 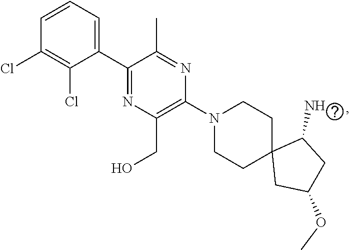 [SMILES: CO[C@H]1C[C@@H](N)C2(CCN(c3nc(C)c(-c4cccc(Cl)c4Cl)nc3CO)CC2)C1] |
| B-171 | 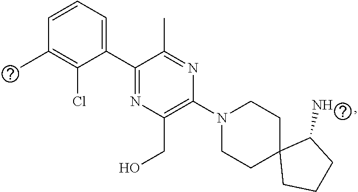 [SMILES: Cc1cccc(-c2nc(CO)c(N3CCC4(CCC[C@H]4N)CC3)nc2C)c1Cl] |
| B-172 | 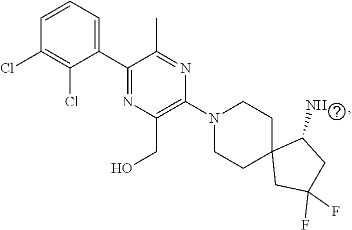 [SMILES: Cc1nc(N2CCC3(CC2)CC(F)(F)C[C@H]3N)c(CO)nc1-c1cccc(Cl)c1Cl] |
| B-173 | 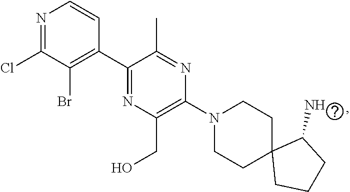 [SMILES: Cc1nc(N2CCC3(CCC[C@H]3N)CC2)c(CO)nc1-c1ccnc(Cl)c1Br] |
| B-174 | 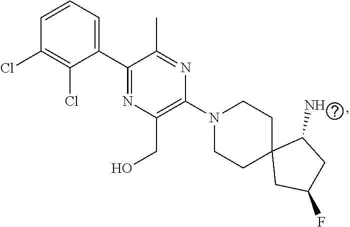 [SMILES: Cc1nc(N2CCC3(CC2)C[C@H](F)C[C@H]3N)c(CO)nc1-c1cccc(Cl)c1Cl] |
| B-175 | 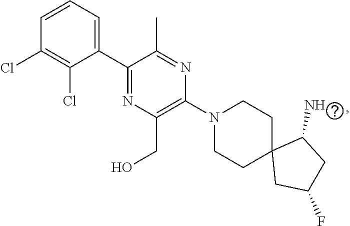 [SMILES: Cc1nc(N2CCC3(CC2)C[C@@H](F)C[C@H]3N)c(CO)nc1-c1cccc(Cl)c1Cl] |
| B-176 | 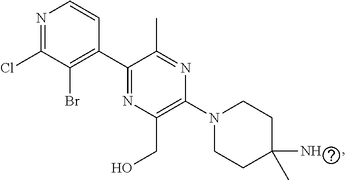 [SMILES: Cc1nc(N2CCC(C)(N)CC2)c(CO)nc1-c1ccnc(Cl)c1Br] |
| B-177 | 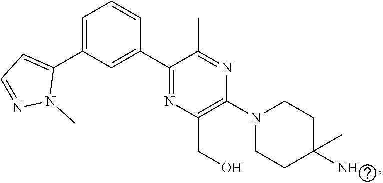 [SMILES: Cc1nc(N2CCC(C)(N)CC2)c(CO)nc1-c1cccc(-c2ccnn2C)c1] |
| B-178 | 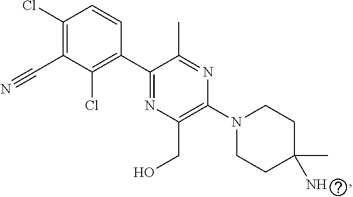 [SMILES: Cc1nc(N2CCC(C)(N)CC2)c(CO)nc1-c1ccc(Cl)c(C#N)c1Cl] |
| B-179 | 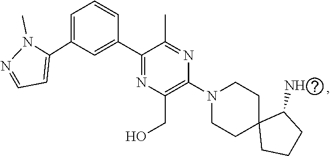 [SMILES: Cc1nc(N2CCC3(CCC[C@H]3N)CC2)c(CO)nc1-c1cccc(-c2ccnn2C)c1] |
| B-180 | 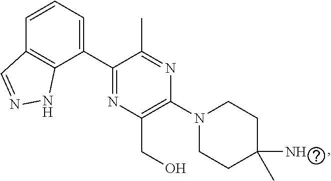 [SMILES: Cc1nc(N2CCC(C)(N)CC2)c(CO)nc1-c1cccc2cn[nH]c12] |
| B-181 | 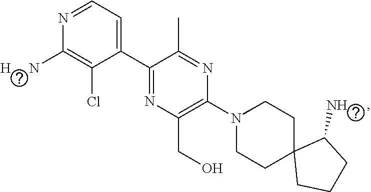 |
| B-182 | 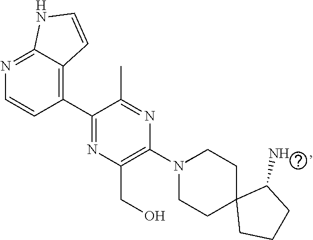 |
| B-183 | 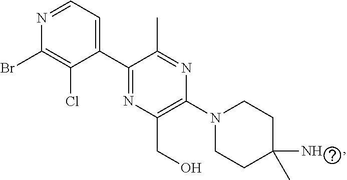 [SMILES: Cc1nc(N2CCC(C)(N)CC2)c(CO)nc1-c1ccnc(Br)c1Cl] |
| B-184 | 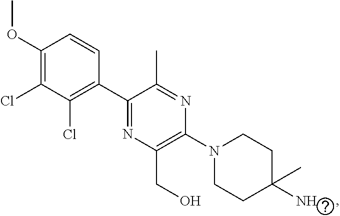 [SMILES: COc1ccc(-c2nc(CO)c(N3CCC(C)(N)CC3)nc2C)c(Cl)c1Cl] |
| B-185 | 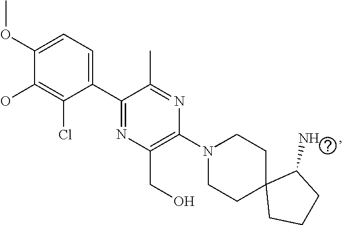 |
| B-186 | 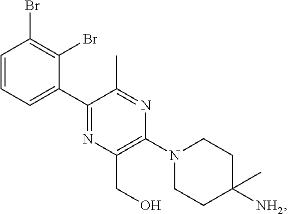 [SMILES: Cc1nc(N2CCC(C)(N)CC2)c(CO)nc1-c1cccc(Br)c1Br] |
| B-187 | 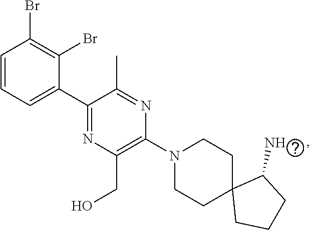 [SMILES: Cc1nc(N2CCC3(CCC[C@H]3N)CC2)c(CO)nc1-c1cccc(Br)c1Br] |
| B-188 | 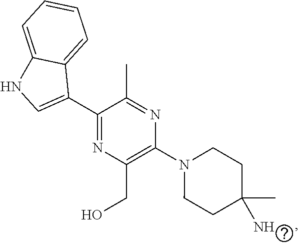 [SMILES: Cc1nc(N2CCC(C)(N)CC2)c(CO)nc1-c1c[nH]c2ccccc12] |
| B-189 | 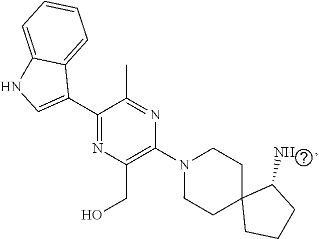 [SMILES: Cc1nc(N2CCC3(CCC[C@H]3N)CC2)c(CO)nc1-c1c[nH]c2ccccc12] |
| B-190 | 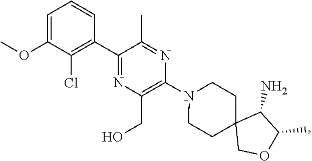 |
| B-191 | 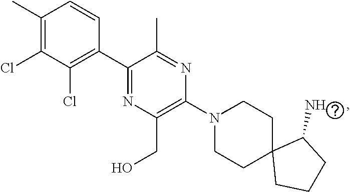 |
| B-192 | 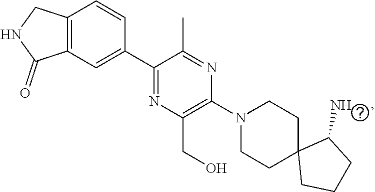 |
| B-193 | 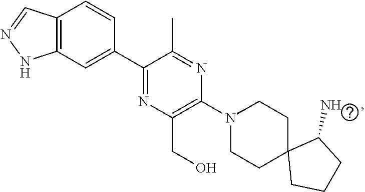 |
| B-194 | 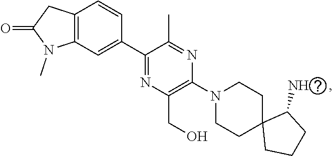 [SMILES: Cc1nc(N2CCC3(CCC[C@H]3N)CC2)c(CO)nc1-c1ccc2c(c1)N(C)C(=O)C2] |
| B-195 | 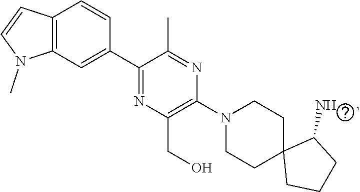 |
| B-196 | 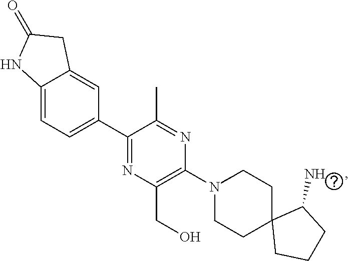 |
| B-197 | 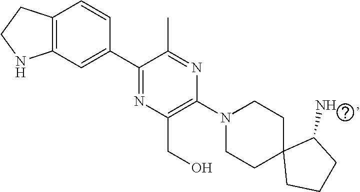 |
| B-198 | 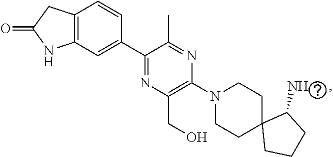 [SMILES: Cc1nc(N2CCC3(CCC[C@H]3N)CC2)c(CO)nc1-c1ccc2c(c1)NC(=O)C2] |
| B-199 | 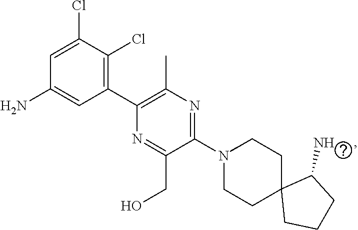 |
| B-200 | 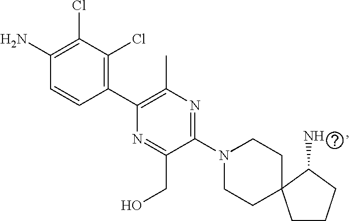 |
| B-201 | 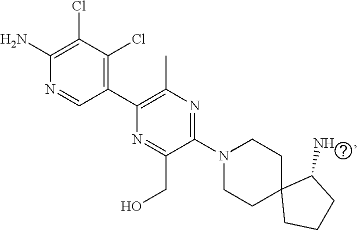 |
| B-202 | 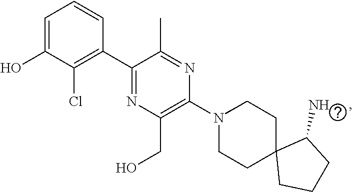 |
| B-203 | 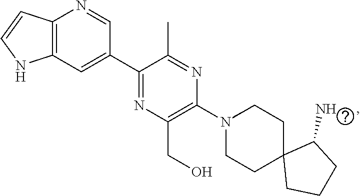 |
| B-204 | 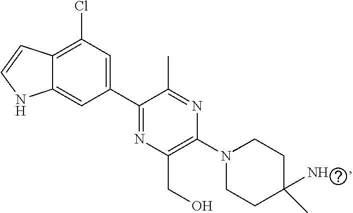 |
| B-205 | 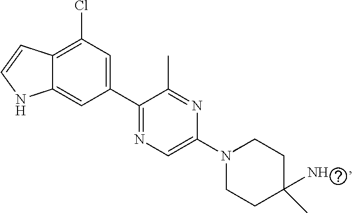 |
| B-206 | 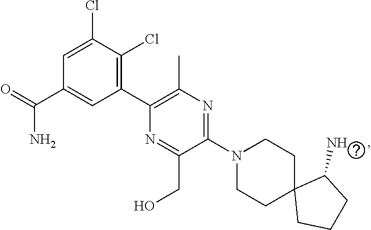 |
| B-207 | 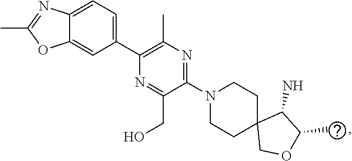 |
| B-208 | 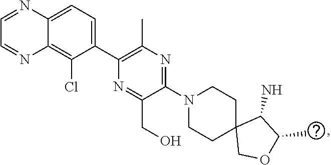 [SMILES: Cc1nc(N2CCC3(CC2)CO[C@@H](C)[C@H]3N)c(CO)nc1-c1ccc2nccnc2c1Cl] |
| B-209 | 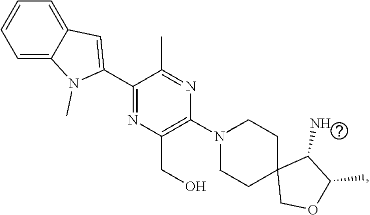 |
| B-210 | 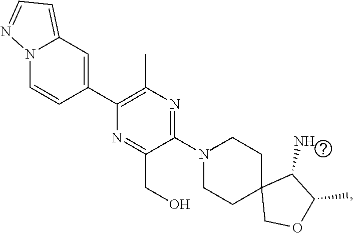 |
| B-211 | 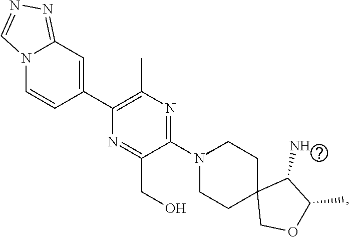 |
| B-212 | 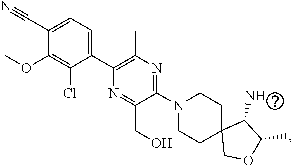 |
| B-213 | 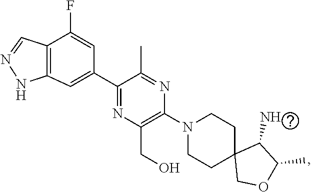 [SMILES: Cc1nc(N2CCC3(CC2)CO[C@@H](C)[C@H]3N)c(CO)nc1-c1cc(F)c2cn[nH]c2c1] |
| B-214 | 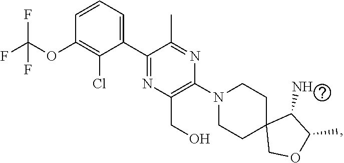 [SMILES: Cc1nc(N2CCC3(CC2)CO[C@@H](C)[C@H]3N)c(CO)nc1-c1cccc(OC(F)(F)F)c1Cl] |
| B-215 | 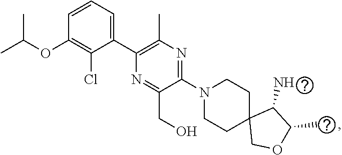 [SMILES: Cc1nc(N2CCC3(CC2)CO[C@@H](C)[C@H]3N)c(CO)nc1-c1cccc(OC(C)C)c1Cl] |
| B-216 | 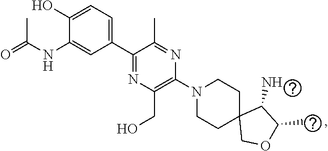 [SMILES: CC(=O)Nc1cc(-c2nc(CO)c(N3CCC4(CC3)CO[C@@H](C)[C@H]4N)nc2C)ccc1O] |
| B-217 | 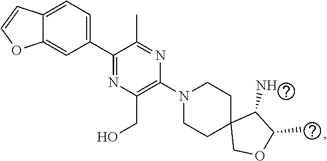 [SMILES: Cc1nc(N2CCC3(CC2)CO[C@@H](C)[C@H]3N)c(CO)nc1-c1ccc2ccoc2c1] |
| B-218 | 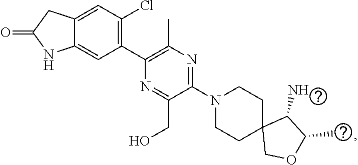 [SMILES: Cc1nc(N2CCC3(CC2)CO[C@@H](C)[C@H]3N)c(CO)nc1-c1cc2c(cc1Cl)CC(=O)N2] |
| B-219 | 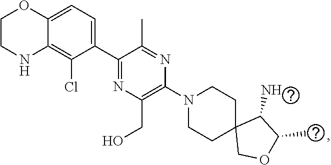 [SMILES: Cc1nc(N2CCC3(CC2)CO[C@@H](C)[C@H]3N)c(CO)nc1-c1ccc2c(c1Cl)NCCO2] |
| B-220 | 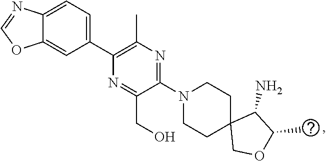 [SMILES: Cc1nc(N2CCC3(CC2)CO[C@@H](C)[C@H]3N)c(CO)nc1-c1ccc2ncoc2c1] |
| B-221 | 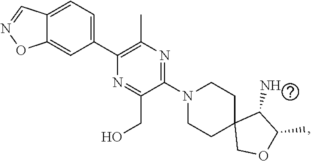 [SMILES: Cc1nc(N2CCC3(CC2)CO[C@@H](C)[C@H]3N)c(CO)nc1-c1ccc2cnoc2c1] |
| B-222 | 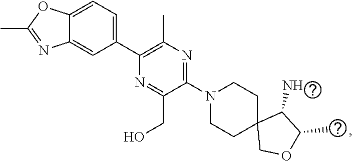 |
| B-223 | 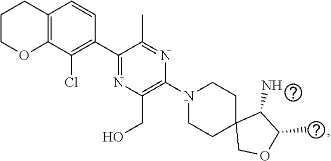 [SMILES: Cc1nc(N2CCC3(CC2)CO[C@@H](C)[C@H]3N)c(CO)nc1-c1ccc2c(c1Cl)OCCC2] |
| B-224 | 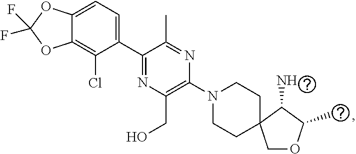 [SMILES: Cc1nc(N2CCC3(CC2)CO[C@@H](C)[C@H]3N)c(CO)nc1-c1ccc2c(c1Cl)OC(F)(F)O2] |
| B-225 | 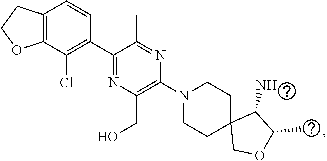 [SMILES: Cc1nc(N2CCC3(CC2)CO[C@@H](C)[C@H]3N)c(CO)nc1-c1ccc2c(c1Cl)OCC2] |
| B-226 | 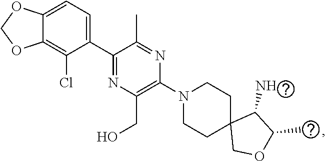 [SMILES: Cc1nc(N2CCC3(CC2)CO[C@@H](C)[C@H]3N)c(CO)nc1-c1ccc2c(c1Cl)OCO2] |
| B-227 | 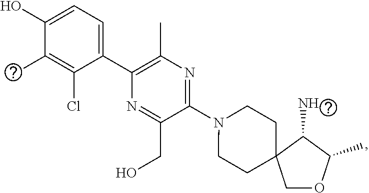 |
| B-228 | 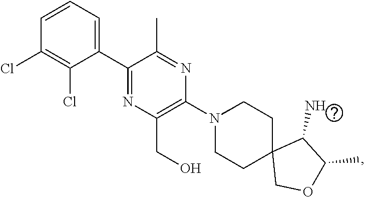 |
| B-229 | 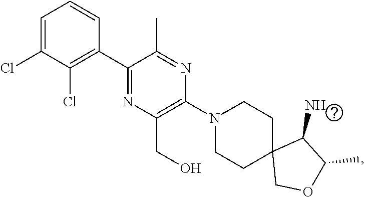 |
| B-230 | 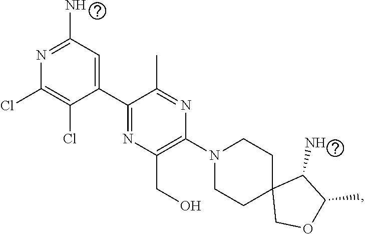 [SMILES: Cc1nc(N2CCC3(CC2)CO[C@@H](C)[C@H]3N)c(CO)nc1-c1cc(N)nc(Cl)c1Cl] |
| B-231 | 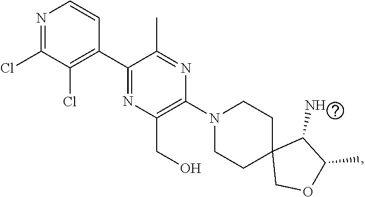 [SMILES: Cc1nc(N2CCC3(CC2)CO[C@@H](C)[C@H]3N)c(CO)nc1-c1ccnc(Cl)c1Cl] |
| B-232 | 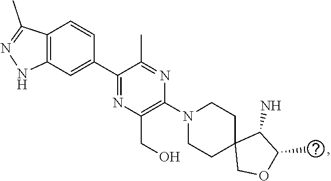 [SMILES: Cc1nc(N2CCC3(CC2)CO[C@@H](C)[C@H]3N)c(CO)nc1-c1ccc2c(C)n[nH]c2c1] |
| B-233 | 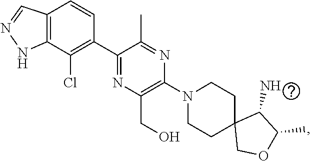 [SMILES: Cc1nc(N2CCC3(CC2)CO[C@@H](C)[C@H]3N)c(CO)nc1-c1ccc2cn[nH]c2c1Cl] |
| B-234 | 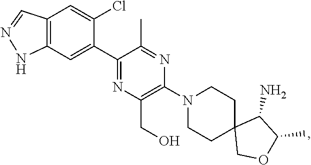 [SMILES: Cc1nc(N2CCC3(CC2)CO[C@@H](C)[C@H]3N)c(CO)nc1-c1cc2[nH]ncc2cc1Cl] |
| B-235 | 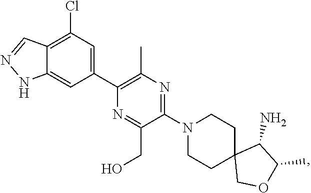 [SMILES: Cc1nc(N2CCC3(CC2)CO[C@@H](C)[C@H]3N)c(CO)nc1-c1cc(Cl)c2cn[nH]c2c1] |
| B-236 | 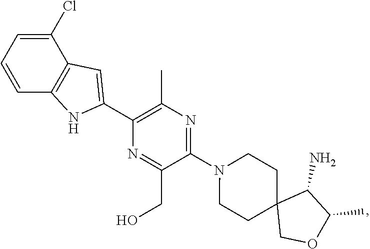 [SMILES: Cc1nc(N2CCC3(CC2)CO[C@@H](C)[C@H]3N)c(CO)nc1-c1cc2c(Cl)cccc2[nH]1] |
| B-237 | 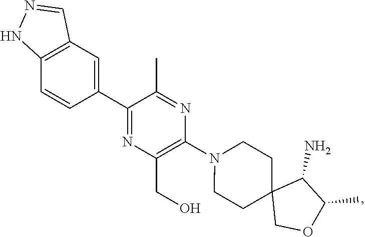 [SMILES: Cc1nc(N2CCC3(CC2)CO[C@@H](C)[C@H]3N)c(CO)nc1-c1ccc2[nH]ncc2c1] |
| B-238 | 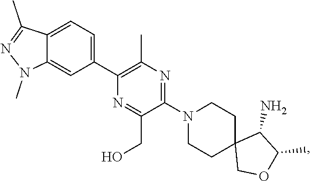 [SMILES: Cc1nc(N2CCC3(CC2)CO[C@@H](C)[C@H]3N)c(CO)nc1-c1ccc2c(C)nn(C)c2c1] |
| B-239 | 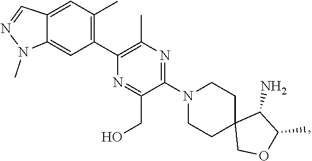 [SMILES: Cc1cc2cnn(C)c2cc1-c1nc(CO)c(N2CCC3(CC2)CO[C@@H](C)[C@H]3N)nc1C] |
| B-240 | 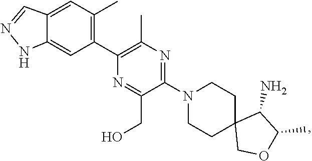 [SMILES: Cc1cc2cn[nH]c2cc1-c1nc(CO)c(N2CCC3(CC2)CO[C@@H](C)[C@H]3N)nc1C] |
| B-241 | 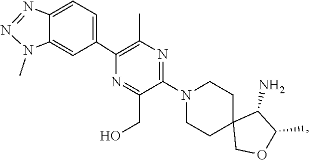 [SMILES: Cc1nc(N2CCC3(CC2)CO[C@@H](C)[C@H]3N)c(CO)nc1-c1ccc2nnn(C)c2c1] |
| B-242 | 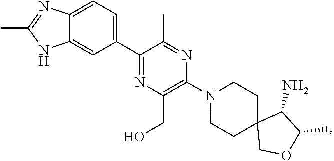 |
| B-243 | 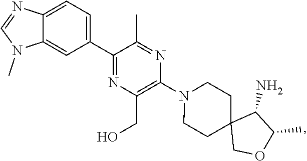 [SMILES: Cc1nc(N2CCC3(CC2)CO[C@@H](C)[C@H]3N)c(CO)nc1-c1ccc2ncn(C)c2c1] |
| B-244 | 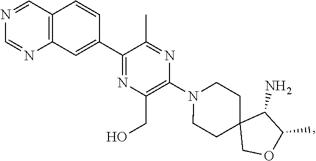 [SMILES: Cc1nc(N2CCC3(CC2)CO[C@@H](C)[C@H]3N)c(CO)nc1-c1ccc2cncnc2c1] |
| B-245 | 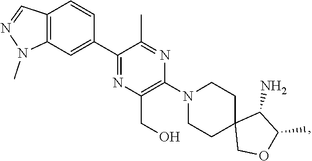 [SMILES: Cc1nc(N2CCC3(CC2)CO[C@@H](C)[C@H]3N)c(CO)nc1-c1ccc2cnn(C)c2c1] |
| B-246 | 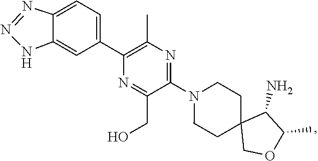 [SMILES: Cc1nc(N2CCC3(CC2)CO[C@@H](C)[C@H]3N)c(CO)nc1-c1ccc2nn[nH]c2c1] |
| B-247 | 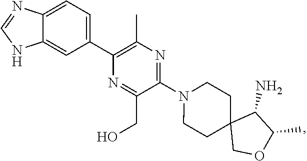 [SMILES: Cc1nc(N2CCC3(CC2)CO[C@@H](C)[C@H]3N)c(CO)nc1-c1ccc2nc[nH]c2c1] |
| B-248 | 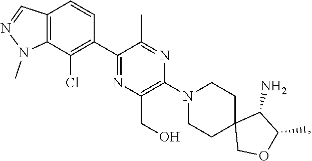 [SMILES: Cc1nc(N2CCC3(CC2)CO[C@@H](C)[C@H]3N)c(CO)nc1-c1ccc2cnn(C)c2c1Cl] |
| B-249 | 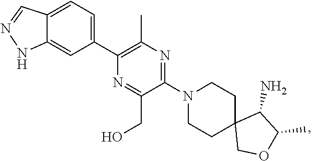 [SMILES: Cc1nc(N2CCC3(CC2)CO[C@@H](C)[C@H]3N)c(CO)nc1-c1ccc2cn[nH]c2c1] |
| B-250 | 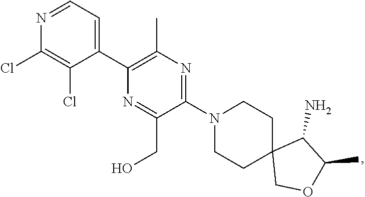 [SMILES: Cc1nc(N2CCC3(CC2)CO[C@H](C)[C@H]3N)c(CO)nc1-c1ccnc(Cl)c1Cl] |
| B-251 | 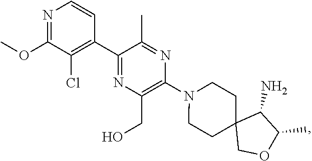 |
| B-252 | 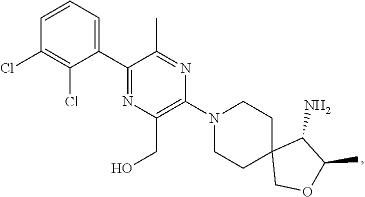 [SMILES: Cc1nc(N2CCC3(CC2)CO[C@H](C)[C@H]3N)c(CO)nc1-c1cccc(Cl)c1Cl] |
| B-253 | 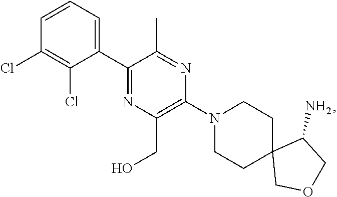 [SMILES: Cc1nc(N2CCC3(CC2)COC[C@H]3N)c(CO)nc1-c1cccc(Cl)c1Cl] |
| B-254 | 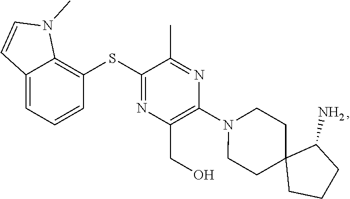 [SMILES: Cc1nc(N2CCC3(CCC[C@H]3N)CC2)c(CO)nc1Sc1cccc2ccn(C)c12] |
| B-255 | 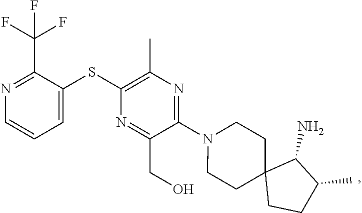 [SMILES: Cc1nc(N2CCC3(CC[C@@H](C)[C@H]3N)CC2)c(CO)nc1Sc1cccnc1C(F)(F)F] |
| B-256 | 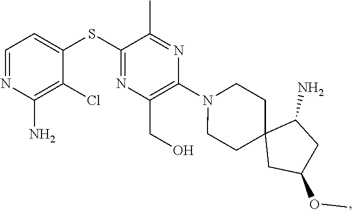 [SMILES: CO[C@@H]1C[C@@H](N)C2(CCN(c3nc(C)c(Sc4ccnc(N)c4Cl)nc3CO)CC2)C1] |
| B-257 | 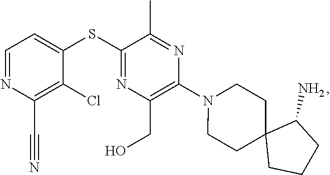 [SMILES: Cc1nc(N2CCC3(CCC[C@H]3N)CC2)c(CO)nc1Sc1ccnc(C#N)c1Cl] |
| B-258 | 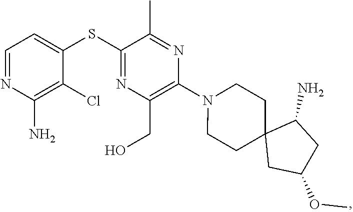 [SMILES: CO[C@H]1C[C@@H](N)C2(CCN(c3nc(C)c(Sc4ccnc(N)c4Cl)nc3CO)CC2)C1] |
| B-259 | 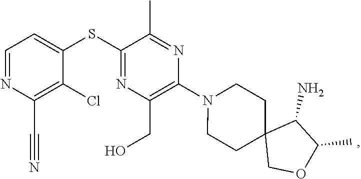 [SMILES: Cc1nc(N2CCC3(CC2)CO[C@@H](C)[C@H]3N)c(CO)nc1Sc1ccnc(C#N)c1Cl] |
| B-260 | 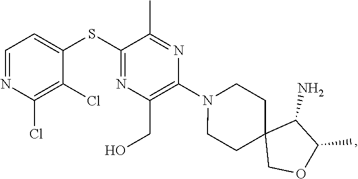 [SMILES: Cc1nc(N2CCC3(CC2)CO[C@@H](C)[C@H]3N)c(CO)nc1Sc1ccnc(Cl)c1Cl] |
| B-261 | 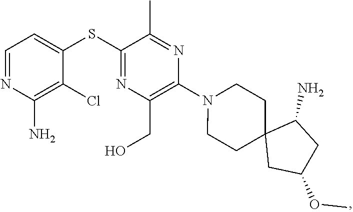 [SMILES: CO[C@H]1C[C@@H](N)C2(CCN(c3nc(C)c(Sc4ccnc(N)c4Cl)nc3CO)CC2)C1] |
| B-262 | 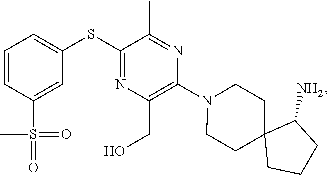 [SMILES: Cc1nc(N2CCC3(CCC[C@H]3N)CC2)c(CO)nc1Sc1cccc(S(C)(=O)=O)c1] |
| B-263 | 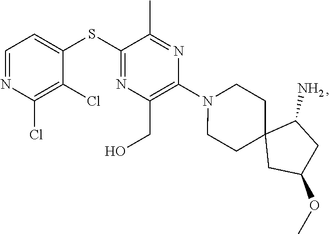 [SMILES: CO[C@@H]1C[C@@H](N)C2(CCN(c3nc(C)c(Sc4ccnc(Cl)c4Cl)nc3CO)CC2)C1] |
| B-264 | 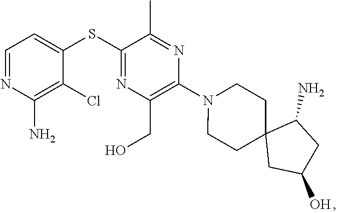 [SMILES: Cc1nc(N2CCC3(CC2)C[C@H](O)C[C@H]3N)c(CO)nc1Sc1ccnc(N)c1Cl] |
| B-265 | 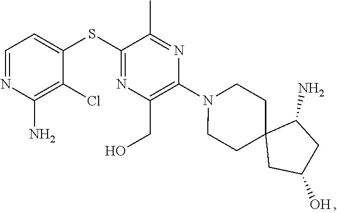 [SMILES: Cc1nc(N2CCC3(CC2)C[C@@H](O)C[C@H]3N)c(CO)nc1Sc1ccnc(N)c1Cl] |
| B-266 | 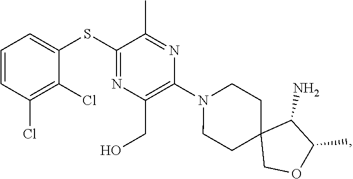 [SMILES: Cc1nc(N2CCC3(CC2)CO[C@@H](C)[C@H]3N)c(CO)nc1Sc1cccc(Cl)c1Cl] |
| B-267 | 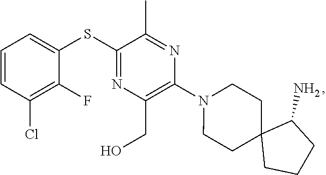 [SMILES: Cc1nc(N2CCC3(CCC[C@H]3N)CC2)c(CO)nc1Sc1cccc(Cl)c1F] |
| B-268 | 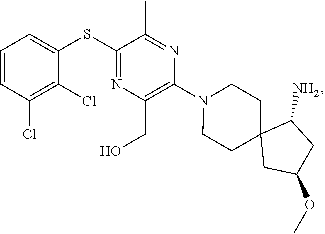 [SMILES: CO[C@@H]1C[C@@H](N)C2(CCN(c3nc(C)c(Sc4cccc(Cl)c4Cl)nc3CO)CC2)C1] |
| B-269 | 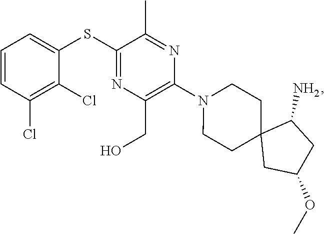 [SMILES: CO[C@H]1C[C@@H](N)C2(CCN(c3nc(C)c(Sc4cccc(Cl)c4Cl)nc3CO)CC2)C1] |
| B-270 | 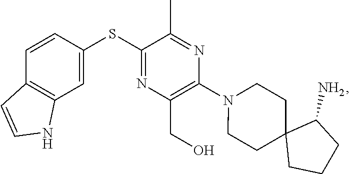 [SMILES: Cc1nc(N2CCC3(CCC[C@H]3N)CC2)c(CO)nc1Sc1ccc2cc[nH]c2c1] |
| B-271 | 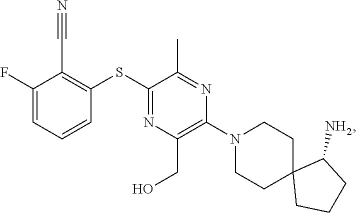 [SMILES: Cc1nc(N2CCC3(CCC[C@H]3N)CC2)c(CO)nc1Sc1cccc(F)c1C#N] |
| B-272 | 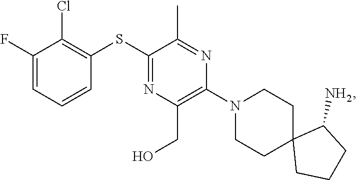 [SMILES: Cc1nc(N2CCC3(CCC[C@H]3N)CC2)c(CO)nc1Sc1cccc(F)c1Cl] |
| B-273 | 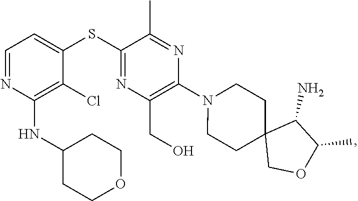 [SMILES: Cc1nc(N2CCC3(CC2)CO[C@@H](C)[C@H]3N)c(CO)nc1Sc1ccnc(NC2CCOCC2)c1Cl] |
| B-274 | 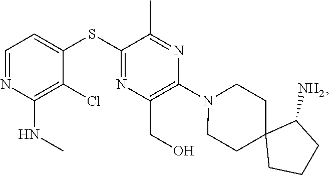 [SMILES: CNc1nccc(Sc2nc(CO)c(N3CCC4(CCC[C@H]4N)CC3)nc2C)c1Cl] |
| B-275 | 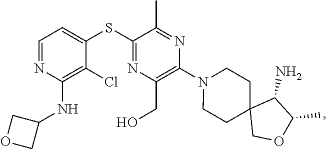 [SMILES: Cc1nc(N2CCC3(CC2)CO[C@@H](C)[C@H]3N)c(CO)nc1Sc1ccnc(NC2COC2)c1Cl] |
| B-276 | 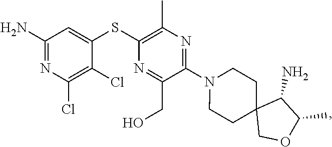 [SMILES: Cc1nc(N2CCC3(CC2)CO[C@@H](C)[C@H]3N)c(CO)nc1Sc1cc(N)nc(Cl)c1Cl] |
| B-277 | 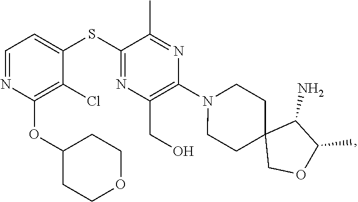 [SMILES: Cc1nc(N2CCC3(CC2)CO[C@@H](C)[C@H]3N)c(CO)nc1Sc1ccnc(OC2CCOCC2)c1Cl] |
| B-278 | 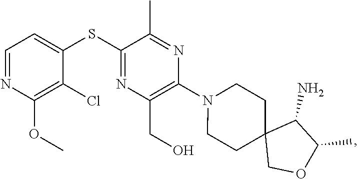 [SMILES: COc1nccc(Sc2nc(CO)c(N3CCC4(CC3)CO[C@@H](C)[C@H]4N)nc2C)c1Cl] |
| B-279 | 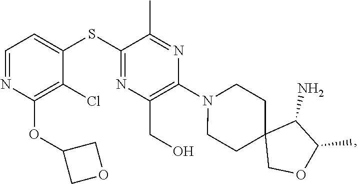 [SMILES: Cc1nc(N2CCC3(CC2)CO[C@@H](C)[C@H]3N)c(CO)nc1Sc1ccnc(OC2COC2)c1Cl] |
| B-280 | 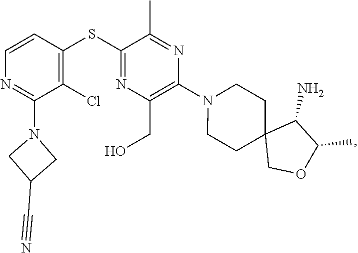 [SMILES: Cc1nc(N2CCC3(CC2)CO[C@@H](C)[C@H]3N)c(CO)nc1Sc1ccnc(N2CC(C#N)C2)c1Cl] |
| B-281 | 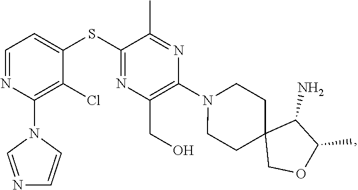 [SMILES: Cc1nc(N2CCC3(CC2)CO[C@@H](C)[C@H]3N)c(CO)nc1Sc1ccnc(-n2ccnc2)c1Cl] |
| B-282 | 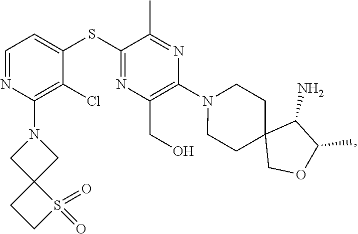 [SMILES: Cc1nc(N2CCC3(CC2)CO[C@@H](C)[C@H]3N)c(CO)nc1Sc1ccnc(N2CC3(CCS3(=O)=O)C2)c1Cl] |
| B-283 | 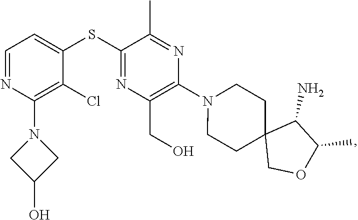 [SMILES: Cc1nc(N2CCC3(CC2)CO[C@@H](C)[C@H]3N)c(CO)nc1Sc1ccnc(N2CC(O)C2)c1Cl] |
| B-284 | 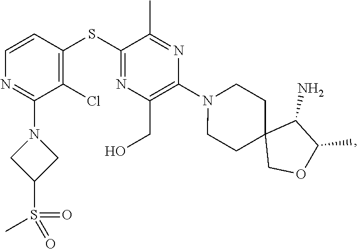 [SMILES: Cc1nc(N2CCC3(CC2)CO[C@@H](C)[C@H]3N)c(CO)nc1Sc1ccnc(N2CC(S(C)(=O)=O)C2)c1Cl] |
| B-285 | 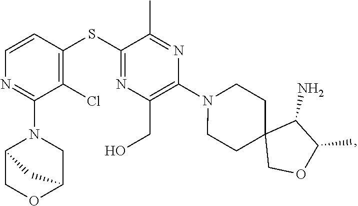 [SMILES: Cc1nc(N2CCC3(CC2)CO[C@@H](C)[C@H]3N)c(CO)nc1Sc1ccnc(N2C[C@@H]3C[C@H]2CO3)c1Cl] |
| B-286 | 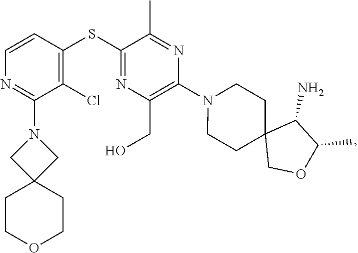 [SMILES: Cc1nc(N2CCC3(CC2)CO[C@@H](C)[C@H]3N)c(CO)nc1Sc1ccnc(N2CC3(CCOCC3)C2)c1Cl] |
| B-287 | 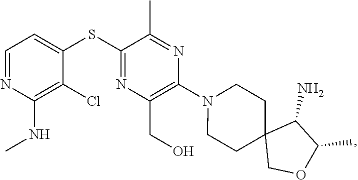 [SMILES: CNc1nccc(Sc2nc(CO)c(N3CCC4(CC3)CO[C@@H](C)[C@H]4N)nc2C)c1Cl] |
| B-288 | 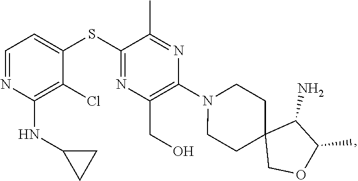 [SMILES: Cc1nc(N2CCC3(CC2)CO[C@@H](C)[C@H]3N)c(CO)nc1Sc1ccnc(NC2CC2)c1Cl] |
| B-289 | 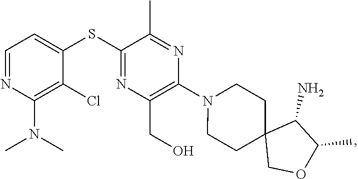 [SMILES: Cc1nc(N2CCC3(CC2)CO[C@@H](C)[C@H]3N)c(CO)nc1Sc1ccnc(N(C)C)c1Cl] |
| B-290 | 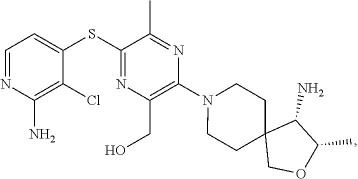 [SMILES: Cc1nc(N2CCC3(CC2)CO[C@@H](C)[C@H]3N)c(CO)nc1Sc1ccnc(N)c1Cl] |
| B-291 | 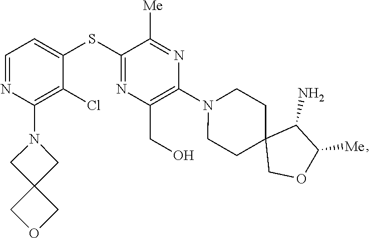 [SMILES: Cc1nc(N2CCC3(CC2)CO[C@@H](C)[C@H]3N)c(CO)nc1Sc1ccnc(N2CC3(COC3)C2)c1Cl] |
| B-292 | 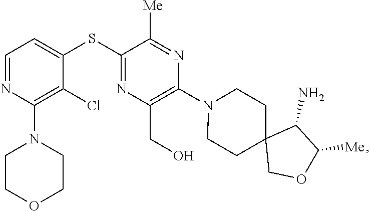 [SMILES: Cc1nc(N2CCC3(CC2)CO[C@@H](C)[C@H]3N)c(CO)nc1Sc1ccnc(N2CCOCC2)c1Cl] |
| B-293 | 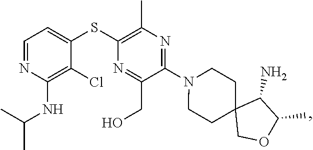 [SMILES: Cc1nc(N2CCC3(CC2)CO[C@@H](C)[C@H]3N)c(CO)nc1Sc1ccnc(NC(C)C)c1Cl] |
| B-294 | 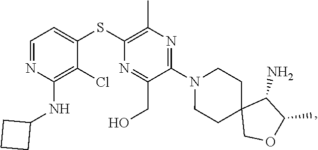 [SMILES: Cc1nc(N2CCC3(CC2)CO[C@@H](C)[C@H]3N)c(CO)nc1Sc1ccnc(NC2CCC2)c1Cl] |
| B-295 | 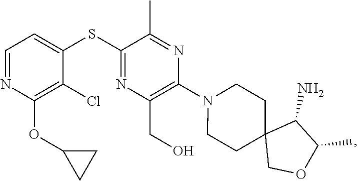 [SMILES: Cc1nc(N2CCC3(CC2)CO[C@@H](C)[C@H]3N)c(CO)nc1Sc1ccnc(OC2CC2)c1Cl] |
| B-296 | 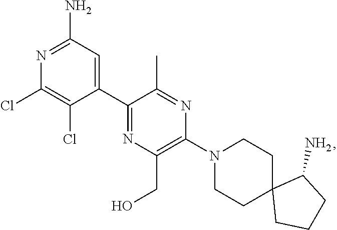 [SMILES: Cc1nc(N2CCC3(CCC[C@H]3N)CC2)c(CO)nc1-c1cc(N)nc(Cl)c1Cl] |
| B-297 | 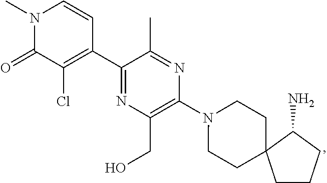 [SMILES: Cc1nc(N2CCC3(CCC[C@H]3N)CC2)c(CO)nc1-c1ccn(C)c(=O)c1Cl] |
| B-298 | 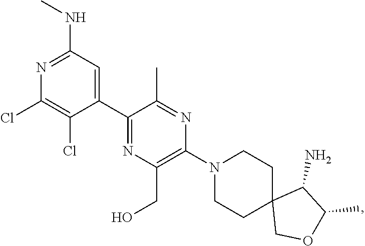 [SMILES: CNc1cc(-c2nc(CO)c(N3CCC4(CC3)CO[C@@H](C)[C@H]4N)nc2C)c(Cl)c(Cl)n1] |
| B-299 | 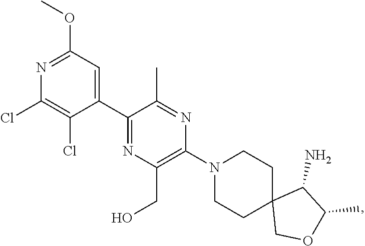 [SMILES: COc1cc(-c2nc(CO)c(N3CCC4(CC3)CO[C@@H](C)[C@H]4N)nc2C)c(Cl)c(Cl)n1] |
| B-300 | 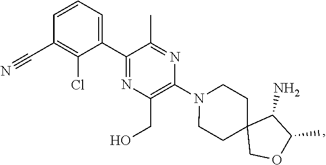 [SMILES: Cc1nc(N2CCC3(CC2)CO[C@@H](C)[C@H]3N)c(CO)nc1-c1cccc(C#N)c1Cl] |
| B-301 | 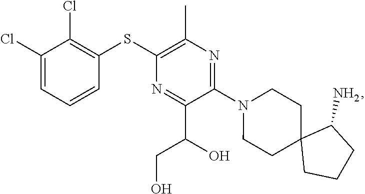 [SMILES: Cc1nc(N2CCC3(CCC[C@H]3N)CC2)c(C(O)CO)nc1Sc1cccc(Cl)c1Cl] |
| B-302 | 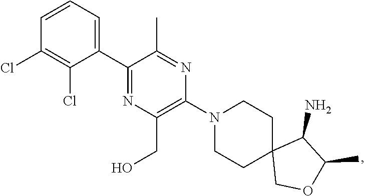 [SMILES: Cc1nc(N2CCC3(CC2)CO[C@H](C)[C@@H]3N)c(CO)nc1-c1cccc(Cl)c1Cl] |
| B-303 | 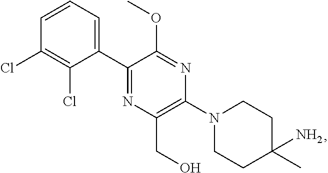 [SMILES: COc1nc(N2CCC(C)(N)CC2)c(CO)nc1-c1cccc(Cl)c1Cl] |
| B-304 | 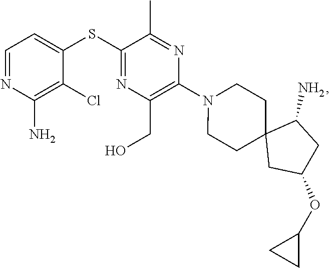 [SMILES: Cc1nc(N2CCC3(CC2)C[C@@H](OC2CC2)C[C@H]3N)c(CO)nc1Sc1ccnc(N)c1Cl] |
| B-305 | 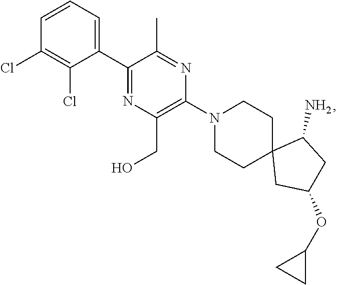 [SMILES: Cc1nc(N2CCC3(CC2)C[C@@H](OC2CC2)C[C@H]3N)c(CO)nc1-c1cccc(Cl)c1Cl] |
| B-306 | 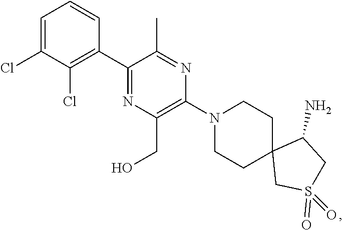 [SMILES: Cc1nc(N2CCC3(CC2)CS(=O)(=O)C[C@H]3N)c(CO)nc1-c1cccc(Cl)c1Cl] |
| B-307 | 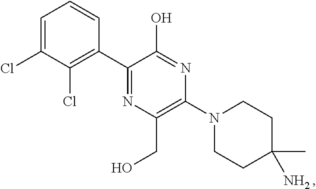 [SMILES: CC1(N)CCN(c2nc(O)c(-c3cccc(Cl)c3Cl)nc2CO)CC1] |
| B-308 | 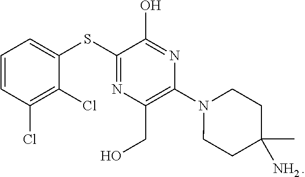 [SMILES: CC1(N)CCN(c2nc(O)c(Sc3cccc(Cl)c3Cl)nc2CO)CC1] |

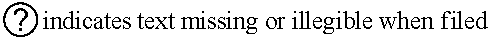

Another aspect of the present disclosure relates to compounds, and pharmaceutically acceptable salts, prodrugs, solvates, hydrates, tautomers, or isomers thereof, in Table A2.

TABLE A2
Structure

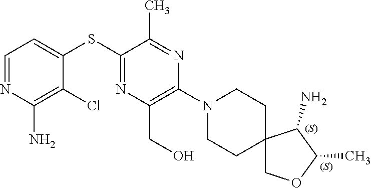

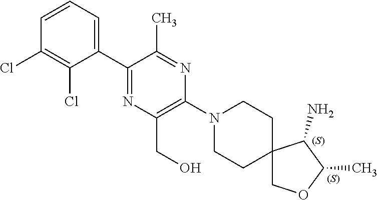

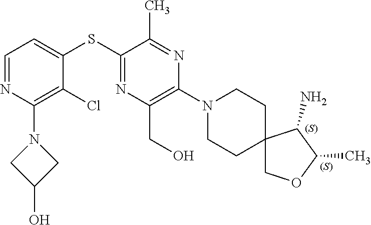

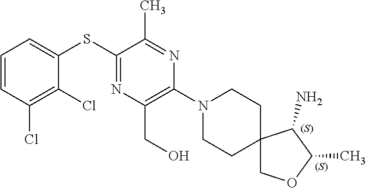

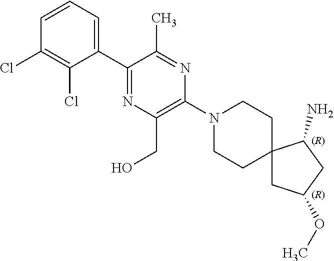

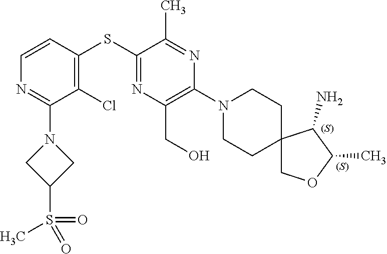

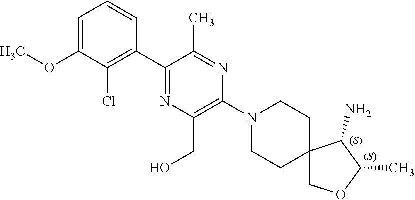

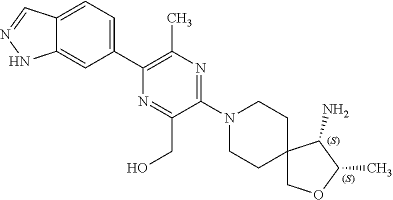

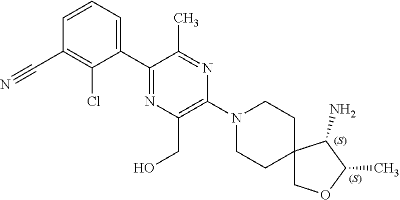

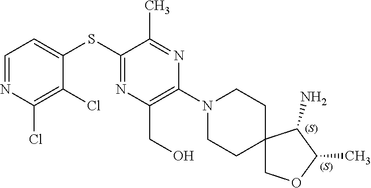

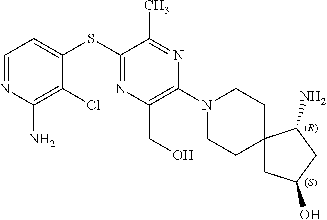

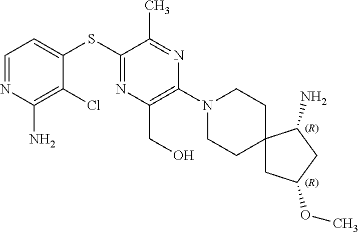

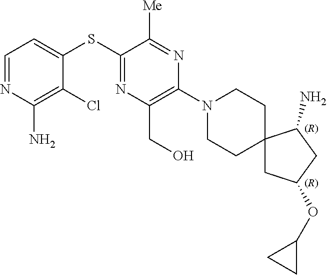

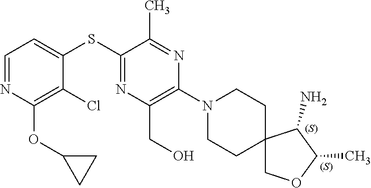

The term “aryl” refers to cyclic, aromatic hydrocarbon groups that have 1 to 2 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, halogen, —O—C-C₆alkyl, —C₁-C₆alkyl, —OC₂-C₆alkenyl, —OC₂-C₆alkynyl, —C₂-C₆alkenyl, —C₂-C₆alkynyl, —OH, —OP(O)(OH)₂, —OC(O)C₁-C₆alkyl, —C(O)C₁-C₆alkyl, —OC(O)OC₁-C₆alkyl, —NH₂, —NH(C₁-C₆alkyl), —N(C₁-C₆alkyl)₂, —S(O)₂—C₁-C₆alkyl, —S(O)NHC₁-C₆alkyl, and —S(O)N(C₁-C₆alkyl)₂. The substituents can themselves be optionally substituted.

Unless otherwise specifically defined, “heteroaryl” means a monovalent or multivalent monocyclic aromatic radical or a polycyclic aromatic radical of 5 to 24 ring atoms, containing one or more ring heteroatoms selected from N, S, P, and O, the remaining ring atoms being C. Heteroaryl as herein defined also means a bicyclic heteroaromatic group wherein the heteroatom is selected from N, S, P, and O. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazolyl, benzo[d]imidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, 1-methyl-1H-indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, dihydrobenzoxanyl, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, isoindolin-1-one, indolin-2-one, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydropyrrolo [1,2-a]pyrimidinyl, 3,4-dihydro-2H-1λ²-pyrrolo [2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, 2-methylbenzo[d]oxazolyl, 1,2,3,4-tetrahydropyrrolo[1,2-a]pyrimidyl, 2,3-dihydrobenzofuranyl, benzooxazolyl, benzoisoxazolyl, benzo[d]isoxazolyl, benzo[d]oxazolyl, furo[2,3-b]pyridinyl, benzothiophenyl, 1,5-naphthyridinyl, furo[3,2-b]pyridinyl, [1,2,4]triazolo[1,5-a]pyridinyl, benzo[1,2,3]triazolyl, 1-methyl-1H-benzo[d][1,2,3]triazolyl, imidazo[1,2-a]pyrimidinyl, [1,2,4] triazolo[4,3-b]pyridazinyl, quinoxalinyl, benzo[c][1,2,5]thiadiazolyl, benzo[c][1,2,5]oxadiazolyl, 1,3-dihydro-2H-benzo[d]imidazol-2-one, 3,4-dihydro-2H-pyrazolo[1,5-b][1,2]oxazinyl, 3,4-dihydro-2H-benzo[b][1,4]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, benzo[d][1,3] dioxolyl, pyrazolo[1,5-a]pyridinyl, and derivatives thereof.

“Alkyl” refers to a straight or branched chain saturated hydrocarbon. C₁-C₆alkyl groups contain 1 to 6 carbon atoms. Examples of a C₁-C₆alkyl group include, but are not limited to, methyl, ethyl, propyl, butyl, pentyl, isopropyl, isobutyl, sec-butyl and tert-butyl, isopentyl and neopentyl.

The term “alkenyl” means an aliphatic hydrocarbon group containing a carbon carbon double bond and which may be straight or branched having about 2 to about 6 carbon atoms in the chain. Certain alkenyl groups have 2 to about 4 carbon atoms in the chain. Branched means that one or more lower alkyl groups such as methyl, ethyl, or propyl are attached to a linear alkenyl chain. Exemplary alkenyl groups include ethenyl, propenyl, n-butenyl, and i-butenyl. A C₂-C₆ alkenyl group is an alkenyl group containing between 2 and 6 carbon atoms.

The term “alkynyl” means an aliphatic hydrocarbon group containing a carbon carbon triple bond and which may be straight or branched having about 2 to about 6 carbon atoms in the chain. Certain alkynyl groups have 2 to about 4 carbon atoms in the chain. Branched means that one or more lower alkyl groups such as methyl, ethyl, or propyl are attached to a linear alkynyl chain. Exemplary alkynyl groups include ethynyl, propynyl, n-butynyl, 2-butynyl, 3-methylbutynyl, and n-pentynyl. A C₂-C₆ alkynyl group is an alkynyl group containing between 2 and 6 carbon atoms.

The term “cycloalkyl” means monocyclic or polycyclic saturated carbon rings containing 3-18 carbon atoms. Examples of cycloalkyl groups include, without limitations, cyclopropyl, cyclobutyl, cyclopentyl, cyclohexyl, cycloheptanyl, cyclooctanyl, norboranyl, norborenyl, bicyclo[2.2.2]octanyl, or bicyclo[2.2.2]octenyl. A C₃-C₈ cycloalkyl is a cycloalkyl group containing between 3 and 8 carbon atoms. A cycloalkyl group can be fused (e.g., decalin) or bridged (e.g., norbornane).

The term “cycloalkenyl” means monocyclic, non-aromatic unsaturated carbon rings containing 4-18 carbon atoms. Examples of cycloalkenyl groups include, without limitation, cyclopentenyl, cyclohexenyl, cycloheptenyl, cyclooctenyl, and norborenyl. A C₄-C₈ cycloalkenyl is a cycloalkenyl group containing between 4 and 8 carbon atoms.

In some embodiments, the terms “heterocyclyl” or “heterocycloalkyl” or “heterocycle” refer to monocyclic or polycyclic 3 to 24-membered rings containing carbon and heteroatoms selected from oxygen, phosphorus, nitrogen, and sulfur and wherein there are no delocalized R electrons (aromaticity) shared among the ring carbon or heteroatoms. Heterocyclyl rings include, but are not limited to, oxetanyl, azetidinyl, tetrahydrofuranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, and homotropanyl. A heteroycyclyl or heterocycloalkyl ring can also be fused or bridged, e.g., can be a bicyclic ring.

In some embodiments “heterocyclyl” or “heterocycloalkyl” or “heterocycle” is a saturated, partially saturated or unsaturated, mono or bicyclic ring containing 3-24 atoms of which at least one atom is chosen from nitrogen, sulfur or oxygen, which may, unless otherwise specified, be carbon or nitrogen linked, wherein a —CH₂— group can optionally be replaced by a —C(O)— or a ring sulfur atom may be optionally oxidised to form the S-oxides. “Heterocyclyl” can be a saturated, partially saturated or unsaturated, mono or bicyclic ring containing 5 or 6 atoms of which at least one atom is chosen from nitrogen, sulfur or oxygen, which may, unless otherwise specified, be carbon or nitrogen linked, wherein a —CH₂— group can optionally be replaced by a —C(O)— or a ring sulfur atom may be optionally oxidised to form S-oxide(s). Non-limiting examples and suitable values of the term “heterocyclyl” are thiazolidinyl, pyrrolidinyl, pyrrolinyl, 2-pyrrolidonyl, 2,5-dioxopyrrolidinyl, 2-benzoxazolinonyl, 1,1-dioxotetrahydro thienyl, 2,4-dioxoimidazolidinyl, 2-oxo-1,3,4-(4-triazolinyl), 2-oxazolidinonyl, 5,6-dihydro uracilyl, 1,3-benzodioxolyl, 1,2,4-oxadiazolyl, 2-azabicyclo[2.2.1]heptyl, 4-thiazolidonyl, morpholino, 2-oxotetrahydrofuranyl, tetrahydrofuranyl, 2,3-dihydrobenzofuranyl, benzothienyl, tetrahydropyranyl, piperidyl, 1-oxo-1,3-dihydroisoindolyl, piperazinyl, thiomorpholino, 1,1-dioxothiomorpholino, tetrahydropyranyl, 1,3-dioxolanyl, homopiperazinyl, thienyl, isoxazolyl, imidazolyl, pyrrolyl, thiadiazolyl, isothiazolyl, 1,2,4-triazolyl, 1,3,4-triazolyl, pyranyl, indolyl, pyrimidyl, thiazolyl, pyrazinyl, pyridazinyl, pyridyl, 4-pyridonyl, quinolyl and 1-isoquinolonyl.

As used herein, the term “halo” or “halogen” means a fluoro, chloro, bromo, or iodo group.

The term “carbonyl” refers to a functional group comprising a carbon atom double-bonded to an oxygen atom. It can be abbreviated herein as “oxo,” as C(O), or as C═O.

“Spirocycle” or “spirocyclic” means carbogenic bicyclic ring systems with both rings connected through a single atom. The ring can be different in size and nature, or identical in size and nature. Examples include spiropentane, spirohexane, spiroheptane, spirooctane, spirononane, or spirodecane. One or both of the rings in a spirocycle can be fused to another carbocyclic, heterocyclic, aromatic, or heteroaromatic ring. One or more of the carbon atoms in the spirocycle can be substituted with a heteroatom (e.g., O, N, S, or P). A C₅-C₁₂ spirocycle is a spirocycle containing between 5 and 12 carbon atoms. In some embodiments, a C₅-C₁₂ spirocycle is a spirocycle containing from 5 to 12 carbon atoms. One or more of the carbon atoms can be substituted with a heteroatom.

The term “spirocyclic heterocycle,” “spiroheterocyclyl,” or “spiroheterocycle” is understood to mean a spirocycle wherein at least one of the rings is a heterocycle (e.g., at least one of the rings is furanyl, morpholinyl, or piperadinyl). A spirocyclic heterocycle can contain between 5 and 12 atoms, at least one of which is a heteroatom selected from N, O, S and P. In some embodiments, a spirocyclic heterocycle can contain from 5 to 12 atoms, at least one of which is a heteroatom selected from N, O, S and P.

The term “tautomers” refers to a set of compounds that have the same number and type of atoms, but differ in bond connectivity and are in equilibrium with one another. A “tautomer” is a single member of this set of compounds. Typically a single tautomer is drawn but it is understood that this single structure is meant to represent all possible tautomers that might exist. Examples include enol-ketone tautomerism. When a ketone is drawn it is understood that both the enol and ketone forms are part of the disclosure.

The SHP2 inhibitor may be administered alone as a monotherapy or in combination with one or more other therapeutic agent (e.g., an inhibitor of a MAP kinase pathway or an anti-cancer therapeutic agent) as a combination therapy. The SHP2 inhibitor may be administered as a pharmaceutical composition. The SHP2 inhibitor may be administered before, after, and/or concurrently with the one or more other therapeutic agent (e.g., an inhibitor of a MAP kinase pathway or an anti-cancer therapeutic agent). If administered concurrently with the one or more other therapeutic agent, such administration may be simultaneous (e.g., in a single composition) or may be via two or more separate compositions, optionally via the same or different modes of administration (e.g., local, systemic, oral, intravenous, etc.).

Administration of the disclosed compositions and compounds (e.g., SHP2 inhibitors and/or other therapeutic agents) can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the disclosed compounds or pharmaceutical compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts. Pharmaceutical compositions suitable for the delivery of a SHP2 inhibitor (alone or, e.g., in combination with another therapeutic agent according to the present disclosure) and methods for their preparation will be readily apparent to those skilled in the art. Such compositions and methods for their preparation may be found, e.g., in Remington's Pharmaceutical Sciences, 19th Edition (Mack Publishing Company, 1995), incorporated herein in its entirety.

Illustrative pharmaceutical compositions are tablets and gelatin capsules comprising a SHP2 inhibitor alone or in combination with another therapeutic agent according to the disclosure and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algiic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, a SHP2 inhibitor (alone or in combination with another therapeutic agent according to the disclosure) is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the SHP2 inhibitor (alone or in combination with another therapeutic agent according to the disclosure).

The SHP2 inhibitor can be also formulated as a suppository, alone or in combination with another therapeutic agent according to the disclosure, which can be prepared from fatty emulsions or suspensions; using polyalkylene glycols such as propylene glycol, as the carrier.

The SHP2 inhibitor can also be administered in the form of liposome delivery systems, such as small unilamellar vesicles, large unilamellar vesicles and multilamellar vesicles, either alone or in combination with another therapeutic agent according to the disclosure. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described for instance in U.S. Pat. No. 5,262,564, the contents of which are hereby incorporated by reference.

SHP2 inhibitors can also be delivered by the use of monoclonal antibodies as individual carriers to which the disclosed compounds are coupled. SHP2 inhibitors can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, a SHP2 inhibitor can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, disclosed compounds are not covalently bound to a polymer, e.g., a polycarboxylic acid polymer, or a polyacrylate.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the invention relates to a pharmaceutical composition comprising a SHP2 inhibitor (alone or in combination with another therapeutic agent according to the present disclosure) and a pharmaceutically acceptable carrier. The pharmaceutically acceptable carrier can further include an excipient, diluent, or surfactant.

Thus, the present disclosure provides compositions (e.g., pharmaceutical compositions) comprising one or more SHP2 inhibitor for use in a method disclosed herein, e.g., a SHP2 monotherapy. Such compositions may comprise a SHP2 inhibitor and, e.g., one or more carrier, excipient, diluent, and/or surfactant.

The present disclosure provides compositions (e.g., pharmaceutical compositions) comprising one or more SHP2 inhibitor and one or more additional therapeutic agent for use in a method disclosed herein, e.g., a SHP2 combination therapy. Such compositions may comprise a SHP2 inhibitor, an additional therapeutic agent (e.g., a TKI, a MAPK pathway inhibitor, an EGFR inhibitor, an ALK inhibitor, a MEK inhibitor) and, e.g., one or more carrier, excipient, diluent, and/or surfactant.

The present disclosure provides compositions (e.g., pharmaceutical compositions) comprising one or more SHP2 inhibitor and one or more MK inhibitor for use in a method disclosed herein, e.g., a SHP2 combination therapy. Such compositions may comprise a SHP2 inhibitor, a MEK inhibitor and, e.g., one or more carrier, excipient, diluent, and/or surfactant. Such compositions may consist essentially of a SHP2 inhibitor, a MEK inhibitor and, e.g., one or more carrier, excipient, diluent, and/or surfactant. Such compositions may consist of a SHP2 inhibitor, a MEK inhibitor and, e.g., one or more carrier, excipient, diluent, and/or surfactant. For example, one non-limiting example of a composition of the present disclosure may comprise, consist essentially of, or consist of (a) a SHP2 inhibitor; (b) a MEK inhibitor selected from one or more of Trametinib (GSK1120212); Selumetinib (AZD6244); Cobimetinib (GDC-0973/XL581), Binimetinib, Vemurafenib, Pimasertib, TAK733, RO4987655 (CH4987655); CI-1040; PD-0325901; Refametinib (RDEA 119/BAY 86-9766); RO5126766, AZD8330 (ARRY-424704/ARRY-704); and GSK1120212; and (c) one or more carrier, excipient, diluent, and/or surfactant. Another non-limiting example of a composition of the present disclosure may comprise, consist essentially of, or consist of (a) a MEK inhibitor; (b) a SHP2 inhibitor selected from (i) RMC-3943; (ii) RMC-4550; (iii) SHP099; (iv) a SHP2 inhibitor compound of any one of Formula I, of Formula II, of Formula III, of Formula I-V1, of Formula I-V2, of Formula I-W, of Formula I-X, of Formula I-Y, of Formula I-Z, of Formula IV, of Formula V, of Formula VI, of Formula IV-X, of Formula IV-Y, of Formula IV-Z, of Formula VII, of Formula VIII, of Formula IX, and of Formula X; (v) TNO155, (vi) a SHP2 inhibitor disclosed in international PCT application PCT/US2017/041577 (WO2018013597), incorporated herein by reference in its entirety; (vii) Compound C; (ix) a compound from Table A1, disclosed herein; (x) a compound from Table A2, disclosed herein; and (xi) a combination thereof, and (c) one or more carrier, excipient, diluent, and/or surfactant.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed RMC-4550 by weight or volume.

The dosage regimen utilizing the disclosed compound is selected in accordance with a variety of factors including type, species, age, weight, sex and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

Effective dosage amounts of a SHP2 inhibitor, when used for the indicated effects, range from about 0.5 mg to about 5000 mg as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. In one embodiment, the compositions are in the form of a tablet that can be scored.

The present invention also provides kits for treating a disease or disorder with a SHP2 inhibitor, one or more carrier, excipient, diluent, and/or surfactant, and a means for determining whether a sample from a subject (e.g., a tumor sample) is likely to be sensitive to SHP2 treatment. In some embodiments, the means for determine comprises a means for determining whether the sample comprises any of an allosteric inhibitor-resistant mutation to SHP2. In some embodiments, the means for determine comprises a means for determining whether the sample comprises any of an allosteric inhibitor-sensitive mutation to SHP2. In some embodiments, the means for determine comprises a means for determining whether the sample comprises any of the following mutations to SHP2: F285S, L262R, S189A, D61G, E69K, T73I, Q506P, E76K, P491S, or S502P. Such means include, but are not limited to direct sequencing, and utilization of a high-sensitivity diagnostic assay (with CE-IVD mark), e.g., as described in Domagala, et al., Pol J Pathol 3: 145-164 (2012), incorporated herein by reference in its entirety, including TheraScreen PCR; AmoyDx; PNAClamp; RealQuality; EntroGen; LightMix; StripAssay; Hybcell plexA; Devyser; Surveyor; Cobas; and TheraScreen Pyro.

All of the U.S. patents, U.S. patent application publications, U.S. patent applications, PCT patent application, PCT patent application publications, foreign patents, foreign patent applications and non-patent publications referred to in this specification or listed in any Application Data Sheet are incorporated herein by reference in their entirety. From the foregoing it will be appreciated that, although specific embodiments of the invention have been described herein for purposes of illustration, various modifications may be made without deviating from the spirit and scope of the invention.

### Example Embodiments

Some embodiments of this disclosure are Example Embodiment I, as follows:

Example Embodiment I-1. A method of treating a subject having a disease or disorder associated with cells containing a mutant SHP2, comprising administering to the subject an allosteric SHP2 inhibitor, wherein the mutant SHP2 comprises an allosteric inhibitor-sensitive mutation.

Example Embodiment I-1a. An allosteric SHP2 inhibitor for use in a method of treating a subject having a disease or disorder associated with cells containing a mutant SHP2, wherein the mutant SHP2 comprises an allosteric inhibitor-sensitive mutation.

Example Embodiment I-1b. Use of an allosteric SHP2 inhibitor for the manufacture of a medicament for treating a subject having a disease or disorder associated with cells containing a mutant SHP2, wherein the mutant SHP2 comprises an allosteric inhibitor-sensitive mutation.

Example Embodiment I-2a. The method of Example Embodiment I-1, wherein the allosteric inhibitor-sensitive mutation is selected from the group consisting of F285S, L262R, S189A, D61G, E69K, T73I, Q506P, and a combination thereof.

Example Embodiment I-2b. The method of Example Embodiment I-1, wherein the allosteric inhibitor-sensitive mutation is selected from the group consisting of F285S, L262R, and S189A.

Example Embodiment I-3. The method of Example Embodiment I-1, wherein the allosteric inhibitor-sensitive mutation is D61G.

Example Embodiment I-4. The method of Example Embodiment I-1, wherein the allosteric inhibitor-sensitive mutation is selected from the group consisting of E69K, T73I, and Q506P.

Example Embodiment I-5. The method of any one of the preceding Example Embodiments, wherein the cells are negative for an allosteric inhibitor-resistant mutation of SHP2.

Example Embodiment I-6a. The method of Example Embodiment I-5, wherein the allosteric inhibitor-resistant mutation is selected from the group consisting of E76K, P491S, S502P, and a combination thereof.

Example Embodiment I-6b. The method of Example Embodiment I-5, wherein the allosteric inhibitor-resistant mutation is selected from the group consisting of E76K and P491S

Example Embodiment I-7. The method of Example Embodiment I-5, wherein the allosteric inhibitor-resistant mutation is S502P.

Example Embodiment I-8. The method of any one of the preceding Example Embodiments, wherein the cells are determined to have the allosteric inhibitor-sensitive mutation prior to administering the SHP2 inhibitor.

Example Embodiment I-9. The method of any one of the preceding Example Embodiments, wherein the cells are determined to not have the allosteric inhibitor-resistant mutation prior to administering the SHP2 inhibitor.

Example Embodiment I-10. The method of any one of the preceding Example Embodiments, wherein the allosteric SHP2 inhibitor is selected from (i) Compound A; (ii) Compound B; (iii) Compound C; (iv) SHP099; (v) an allosteric SHP2 inhibitor compound of any one of Formula I, of Formula II, of Formula III, of Formula I-V1, of Formula I-V2, of Formula I-W, of Formula I-X, of Formula I-Y, of Formula I-Z, of Formula IV, of Formula V, of Formula VI, of Formula IV-X, of Formula IV-Y, of Formula IV-Z, of Formula VII, of Formula VIII, of Formula IX, and of Formula X; (vi) TNO155; (vii) a SHP2 inhibitor disclosed in international PCT application PCT/US2017/041577 (W2018013597), incorporated herein by reference in its entirety; (viii) a compound from Table A1, disclosed herein; (ix) a compound from Table A2, disclosed herein; and (x) a combination thereof.

Example Embodiment I-11. The method of any one of the preceding Example Embodiments, wherein the disease or disorder is selected from tumors of hemopoietic and lymphoid system; a myeloproliferative syndrome; a myelodysplastic syndromes; leukemia; acute myeloid leukemia; juvenile myelomonocytic leukemia; esophageal cancer; breast cancer; lung cancer; colon cancer; gastric cancer; neuroblastoma; bladder cancer; prostate cancer; glioblastoma; urothelial carcinoma; uterine carcinoma; adenoid and ovarian sereous cystadenocarcinoma; paraganglioma; phaeochromocytoma; pancreatic cancer; adrenocortical carcinoma; stomach adenocarcinoma; sarcoma; rhabdomyosarcoma; lymphoma; head and neck cancer; skin cancer; peritoneum cancer; intestinal cancer (e.g., small and/or large intestinal cancer); thyroid cancer; endometrial cancer; cancer of the biliary tract; soft tissue cancer; ovarian cancer; central nervous system cancer (e.g., primary CNS lymphoma); stomach cancer; pituitary cancer; genital tract cancer; urinary tract cancer; salivary gland cancer; cervical cancer; liver cancer; eye cancer; cancer of the adrenal gland; cancer of autonomic ganglia; cancer of the upper aerodigestive tract; bone cancer; testicular cancer; pleura cancer; kidney cancer; penis cancer; parathyroid cancer; cancer of the meninges; vulvar cancer; and melanoma.

Example Embodiment I-12. The method of any one of the preceding Example Embodiments, wherein the disease or disorder is an inherited developmental disorder selected from the group consisting of Noonan Syndrome and LEOPARD Syndrome.

Example Embodiment I-13. The method of any one of any one of the preceding Example Embodiments, wherein the allosteric SHP2 inhibitor is administered in an effective amount.

Example Embodiment I-14. A method of identifying a subject with SHP2 mutations susceptible to a SHP2 inhibitor, comprising genotyping a biological sample from the subject for SHP2 mutations, wherein the subject is identified as susceptible to the SHP2 inhibitor if the SHP2 mutations comprise an allosteric inhibitor-sensitive mutation.

Example Embodiment I-14a. An in vitro method of identifying a subject with SHP2 mutations susceptible to a SHP2 inhibitor, comprising genotyping, via an in vitro assay, a biological sample from the subject for SHP2 mutations, wherein the subject is identified as susceptible to the SHP2 inhibitor if the SHP2 mutations comprise an allosteric inhibitor-sensitive mutation.

Example Embodiment I-14b. An allosteric SHP2 inhibitor for use in a method of treating a subject identified by genotyping as having a disease or disorder with a SHP2 mutation that is susceptible to a SHP2 inhibitor, wherein the subject is identified as susceptible to the SHP2 inhibitor if the SHP2 mutations comprise an allosteric inhibitor-sensitive mutation.

Example Embodiment I-14c. Use of an allosteric SHP2 inhibitor for the manufacture of a medicament for treating a subject identified by genotyping as having a disease or disorder with a SHP2 mutation that is susceptible to a SHP2 inhibitor, wherein the subject is identified as susceptible to the SHP2 inhibitor if the SHP2 mutations comprise an allosteric inhibitor-sensitive mutation.

Example Embodiment I-15a. The method of Example Embodiment I-14, wherein the allosteric inhibitor-sensitive mutation is selected from the group consisting of F285S, L262R, S189A, D61G, E69K, T73I, Q506P, and a combination thereof.

Example Embodiment I-15b. The method of Example Embodiment I-14, wherein the allosteric inhibitor-sensitive mutation is selected from the group consisting of F285S, L262R, and S189A.

Example Embodiment I-16. The method of Example Embodiment I-14, wherein the allosteric inhibitor-sensitive mutation is D61G.

Example Embodiment I-17. The method of Example Embodiment I-14, wherein the allosteric inhibitor-sensitive mutation is selected from the group consisting of E69K, T73I, and Q506P.

Example Embodiment I-18. The method of any one of Example Embodiments I-14 to I-15, wherein the method further comprises identifying the subject as not expressing a SHP2 allosteric inhibitor-resistant mutation.

Example Embodiment I-19, The method of Example Embodiment I-18, wherein the SHP2 allosteric inhibitor-resistant mutation is selected from the group consisting of E76K, P491S, S502P, and a combination thereof.

Example Embodiment I-20, The method of Example Embodiment I-18, wherein the allosteric inhibitor-resistant mutation is selected from the group consisting of E76K and P491S

Example Embodiment I-21, The method of Example Embodiment I-18, wherein the allosteric inhibitor-resistant mutation is S502P.

Example Embodiment I-22. The method of any one of Example Embodiments I-14 to I-21, wherein the allosteric SHP2 inhibitor is selected from (i) Compound A; (ii) Compound B; (iii) Compound C; (iv) SHP099; (v) an allosteric SHP2 inhibitor compound of any one of Formula I, of Formula II, of Formula III, of Formula I-V1, of Formula I-V2, of Formula I-W, of Formula I-X, of Formula I-Y, of Formula I-Z, of Formula IV, of Formula V, of Formula VI, of Formula IV-X, of Formula IV-Y, of Formula IV-Z, of Formula VII, of Formula VIII, of Formula IX, and of Formula X; (vi) TNO155, and (vii) a combination thereof.

Example Embodiment I-23. The method of any one of Example Embodiments I-14 through I-22, wherein the allosteric SHP2 inhibitor is in an effective amount.

Example Embodiment I-24. A method of identifying a subject as resistant to an allosteric SHP2 inhibitor, comprising genotyping a biological sample from the subject for SHP2 mutations, wherein the subject is identified as resistant to the SHP2 inhibitor if the SHP2 mutations comprise an allosteric inhibitor-resistant mutation.

Example Embodiment I-24a. An in vitro method of identifying a subject as resistant to an allosteric SHP2 inhibitor, comprising genotyping, via an in vitro assay, a biological sample from the subject for SHP2 mutations, wherein the subject is identified as resistant to the SHP2 inhibitor if the SHP2 mutations comprise an allosteric inhibitor-resistant mutation.

Example Embodiment I-25a. The method of Example Embodiment I-24, wherein the allosteric inhibitor-resistant mutation is selected from the group consisting of E76K, P491S, S502P, and a combination thereof.

Example Embodiment I-25b. The method of Example Embodiment I-24, wherein the allosteric inhibitor-resistant mutation is selected from the group consisting of E76K and P491S

Example Embodiment I-26. The method of Example Embodiment I-24, wherein the allosteric inhibitor-resistant mutation is S502P.

Example Embodiment I-27. The method of any one of Example Embodiments I-24 to I-26, wherein the allosteric SHP2 inhibitor is selected from (i) Compound A; (ii) Compound B; (iii) Compound C; (iv) SHP099; (v) an allosteric SHP2 inhibitor compound of any one of Formula I, of Formula II, of Formula III, of Formula I-V1, of Formula I-V2, of Formula I-W, of Formula I-X, of Formula I-Y, of Formula I-Z, of Formula IV, of Formula V, of Formula VI, of Formula IV-X, of Formula IV-Y, of Formula IV-Z, of Formula VII, of Formula VIII, of Formula IX, and of Formula X; (vi) TNO155, and (vii) a combination thereof.

Example Embodiment I-28. The method of any one of Example Embodiments I-24 through I-27, wherein the allosteric SHP2 inhibitor is in an effective amount.

Example Embodiment I-29. A diagnostic test for allosteric SHP2 inhibitor sensitivity, comprising a nucleic acid probe specific for an allosteric inhibitor-sensitive mutation of SHP2.

Example Embodiment I-29a. An in vitro diagnostic test for allosteric SHP2 inhibitor sensitivity, comprising a nucleic acid probe specific for an allosteric inhibitor-sensitive mutation of SHP2.

Example Embodiment I-30. The diagnostic test of Example Embodiment I-29, wherein the allosteric inhibitor-sensitive mutation is selected from the group consisting of F285S, L262R, S189A, D61G, E69K, T73I, Q506P, and a combination thereof.

Example Embodiment I-31. The diagnostic test of Example Embodiment I-29, wherein the allosteric inhibitor-sensitive mutation is selected from the group consisting of F285S, L262R, and S189A.

Example Embodiment I-32. The diagnostic test of Example Embodiment I-29, wherein the allosteric inhibitor-sensitive mutation is D61G.

Example Embodiment I-33. The diagnostic test of Example Embodiment I-29, wherein the allosteric inhibitor-sensitive mutation is selected from the group consisting of E69K, T73I, and Q506P.

Example Embodiment I-34. A diagnostic test for allosteric SHP2 inhibitor insensitivity, comprising a nucleic acid probe specific for a SHP2 allosteric inhibitor-resistant mutation; wherein the allosteric inhibitor-resistant mutation is optionally selected from E76K, P491S, S502P.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

Activating Mutations have Differential Effect on Biochemical Potency of Allosteric Inhibitors

SHP2 (PTPN11) is a non-receptor protein tyrosine phosphatase and scaffold protein that functions downstream of multiple RTKs, integrating growth factor signals to promote RAS/MAPK activation. SHP2 is composed of three distinct structural domains: two SH2 domains at the N-terminus followed by a PTP catalytic domain. SHP2 adopts an autoinhibited conformation in the absence of RTK signaling. Mutations that destabilize the autoinhibited conformation are common in inherited RASopathies and certain cancers. Allosteric inhibitors that stabilize the autoinhibited conformation in wild-type SHP2 inhibit RAS/MAPK signaling, and tumor growth, in xenograft models driven by oncogenic mutations in the RAS/MAPK pathway. This study asked what is the effect of allosteric inhibitors on activated mutant SHP2.

Binding to diphosphotyrosine motifs in signaling proteins destabilizes the inhibited state and activates the enzyme. SHP2 can be activated in vitro by synthetic peptides containing diphosphotyrosine motifs. Mutations in the SH2-Catalytic domain interface can uncouple activation from phosphotyrosine peptide or protein binding. Molecules that bind specifically to the autoinhibited conformation function as allosteric inhibitors

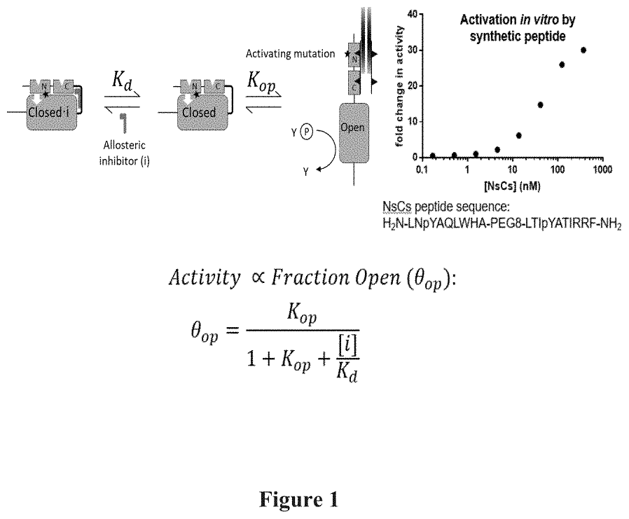

Activation/inhibition by peptide binding, mutation, and inhibitor binding can be described with a simple equilibrium model (FIG. 1).

The present study examined the effect of allosteric inhibitors on mutant SHP2s. The following mutations associated with Noonan Syndrome, Juvenile Myelomonocytic Leukemia (JMML), and other human cancers were selected for further experimental study: D61G, E76K, S189A, L262R, F285S, P491S and S502P. Mutations refer to the SHP2 sequence numbered according to Uniprot Isoform 2 (accession number Q06124-2) (SEQ ID NO: 1).

### Methods

SHP2 Allosteric Inhibition Assay

Full-length SHP2 is allosterically activated through binding of bis-tyrosyl-phorphorylated peptides to its Src Homology 2 (SH2) domains. The latter activation step leads to the release of the auto-inhibitory interface of SHP2, which in turn renders the SHP2 protein tyrosine phosphatase (PTP) active and available for substrate recognition and reaction catalysis. The catalytic activity of SHP2 was monitored using the surrogate substrate DiFMUP in a prompt fluorescence assay format. Mutant variants of SHP2 showed variable response to activating peptide, and the biochemical assay was repeated on all enzymes with and without activating peptide at a concentration of 500 nM.

The phosphatase reactions were performed at room temperature in 384-well black polystyrene plates, flat bottom, non-binding surface (Corning, Cat #781077) using a final reaction volume of 50 μL and the following assay buffer conditions: 55 mM HEPES pH 7.2, 100 mM NaCl, 0.5 mM EDTA, 1 mM DTT, 0.001% Brij35, 0.002% BSA, 0.1% DMSO, 100 μM DiFMUP, 0.1, 0.3, or 2 nM enzyme, 0 or 500 nM activating peptide NsCs and 10 μM to 1.9 μM inhibitor.

Diluted inhibitor (5 μL) was mixed with activated enzyme (25 μl) and incubated for 30 minutes at room temperature. A 250 μM aqueous DifMUP solution (20 μl) was added and the plate was sealed and incubated for 30 minutes. 50 μl stop solution (0.1 mM sodium pervanadate) was added to each well, the plate was shaken briefly to mix, and read in endpoint mode on a SpectraMax M5 plate reader (Molecular Devices) using excitation and emission wavelengths of 340 nm and 450 nm. Data was imported into GraphPad Prism. Plots of fluorescence intensity vs. log Molar [compound] were created and modeled with a 3-parameter sigmoidal concentration response equation in order to estimate IC₅₀.

### Results

Compound C (also known as Compound 33 on Tables 1-8) and 52 other allosteric inhibitors of SHP2 were tested for their potency in a biochemical assay of SHP2 activity. In this assay, wildtype or mutant variants of SHP2 were incubated with each of compounds 1-53 for 30 minutes, prior to addition of the small molecule substrate DiFMUP (6,8-difluoro-4-methylumbelliferyl phosphate). Reactions were then allowed to proceed for 30 minutes and stopped by the addition of a phosphatase inhibitor, sodium pervanadate. De-phosphorylation of DiFMUP results in production of a fluorescent product. Product fluorescence was determined and plotted as a function of compound concentration in order to determine the IC₅₀ for each compound on each mutant using a four parameter sigmoidal dose response function in Prism (GraphPad).

The experiments were repeated in the presence of a bis-phosphorylated activating peptide (termed “NsCs”) which comprises two tyrosine phosphorylated 9-mers (synthetic sequences designed to strongly bind both the N- and C-terminal SH2 domains) connected by a PEG8 linker. NsCs mimics the role of the cytosolic domain of a protein tyrosine kinase in this model system. The NsCs activating peptide has the following structure:

(SEQ ID NOS: 2-3)
H2N-Leu-Asn-pTyr-Ala-Gln-Leu-Trp-His-Ala-PEG8-Leu-
Thr-Ile-pTyr-Ala-Thr-Ile-Arg-Arg-Phe-NH2.

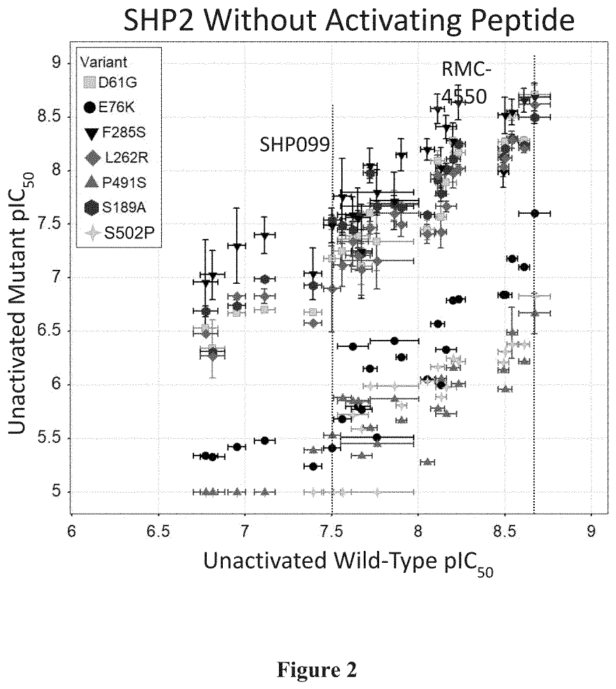

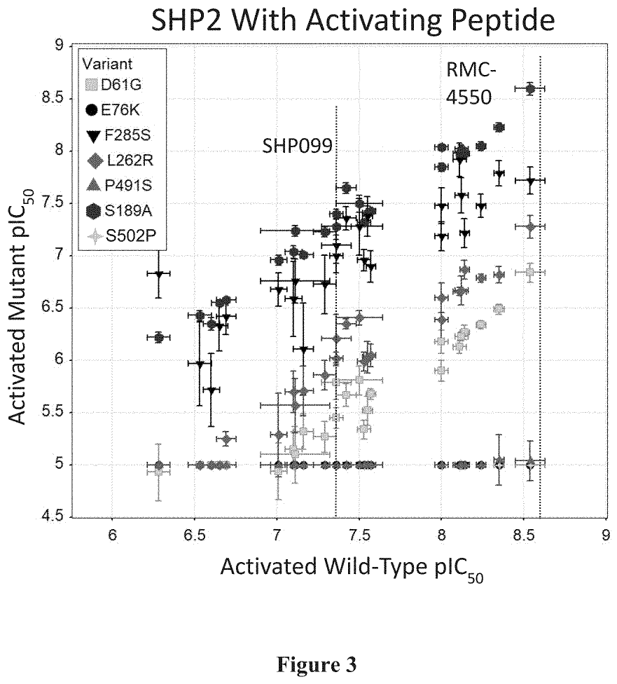

The potencies of 52 compounds to inhibit the non-activated (apo) and activated forms of the various mutants, in comparison to the wild type SHP2, are summarized in Tables 1 to 8. The potency of each compound to inhibit non-activated mutant SHP2 is plotted versus the potency to inhibit wild-type SHP2 in FIG. 2. The same plot for activated mutant and wild-type SHP2 is shown in FIG. 3.

TABLE 1
Biochemical potency (pIC₅₀) for selected SHP2 inhibitors on wild type SHP2 alone
(Non-Activated) and in presence of 0.5 μM NsCs peptide (Activated)

| | Potency of compounds for inhibition of human SHP2-FL wild type | | | | | |
|---|---|---|---|---|---|---|
| | Wild-type SHP2 | | | Wild-type SHP2 | | |
| | No Peptide Control | | | 0.5 μM NsCs peptide | | |
| | (Non-Activated) | | | (Activated) | | |
| | | Standard | IC₅₀ | | Standard | IC₅₀ |
| Compound | pIC₅₀ | Error^{†} | (nM)* | pIC₅₀ | Error^{†} | (nM)* |
| 1* | 8.7 | 0.12 | 2.01 | 8.58 | 0.16 | 2.62 |
| 1 | 8.61 | 0.12 | 2.43 | 8.57 | 0.07 | 2.69 |
| 1 | 8.87 | 0.08 | 1.36 | 8.67 | 0.08 | 2.12 |
| 1 | 8.65 | 0.11 | 2.24 | 8.63 | 0.08 | 2.37 |
| 2 | 7.56 | 0.08 | 27.8 | 7.1 | 0.05 | 79.4 |
| 3 | 8.13 | 0.08 | 7.48 | 7.55 | 0.03 | 28.1 |
| 4 | 7.65 | 0.05 | 22.4 | 7.29 | 0.07 | 50.9 |
| 5 | 7.5 | 0.08 | 31.6 | 7.01 | 0.05 | 98.9 |
| 6 | 7.64 | 0.1 | 22.7 | 7.39 | 0.08 | 40.8 |
| 7 | 8.02 | 0.08 | 9.59 | 7.76 | 0.05 | 17.3 |
| 8 | 8.49 | 0.1 | 3.23 | 8.14 | 0.03 | 7.23 |
| 9 | 7.39 | 0.19 | 40.6 | 6.65 | 0.05 | 225 |
| 10* | 7.82 | 0.1 | 15.1 | 7.59 | 0.09 | 25.9 |
| 10 | 8.01 | 0.06 | 9.77 | 7.59 | 0.13 | 25.9 |
| 11 | 7.62 | 0.12 | 24 | 7.36 | 0.09 | 43.4 |
| 12 | 8.16 | 0.07 | 7 | 7.42 | 0.06 | 38.2 |
| 13 | 8.56 | 0.07 | 2.77 | 8.2 | 0.04 | 6.27 |
| 14 | 7.76 | 0.15 | 17.6 | 7.11 | 0.21 | 78.5 |
| 15 | 7.67 | 0.12 | 21.5 | 7.16 | 0.06 | 69.2 |
| 16 | 8.2 | 0.13 | 6.34 | 8 | 0.04 | 9.91 |
| 17 | 7.11 | 0.11 | 77.6 | 6.69 | 0.06 | 205 |
| 18 | 6.99 | 0.19 | 102 | 6.51 | 0.07 | 310 |
| 19 | 6.33 | 0.26 | 467 | 5.99 | 0.09 | 1030 |
| 20 | 8.85 | 0.1 | 1.42 | 8.85 | 0.07 | 1.42 |
| 21 | 8.67 | 0.10 | 2.16 | 8.54 | 0.09 | 2.89 |
| 22 | 7.31 | 0.25 | 49.3 | 6.81 | 0.06 | 157 |
| 23 | 7.72 | 0.16 | 19.1 | 7.36 | 0.04 | 44.1 |
| 24 | 8.42 | 0.08 | 3.81 | 8.46 | 0.07 | 3.48 |
| 25 | 7.61 | 0.11 | 24.3 | 7.26 | 0.07 | 55 |
| 26 | 7.86 | 0.08 | 13.8 | 7.5 | 0.14 | 31.4 |
| 27 | 8.11 | 0.12 | 7.71 | 7.82 | 0.09 | 15 |
| 28 | 8.61 | 0.09 | 2.48 | 8.24 | 0.03 | 5.81 |
| 29 | 8.54 | 0.09 | 2.89 | 8.35 | 0.03 | 4.44 |
| 30 | 8.23 | 0.08 | 5.87 | 8.11 | 0.04 | 7.83 |
| 31 | 6.77 | 0.1 | 169 | 6.53 | 0.07 | 294 |
| 32 | 8.42 | 0.06 | 3.78 | 8.5 | 0.03 | 3.18 |
| 33 | 8.4 | 0.08 | 4.02 | 8.66 | 0.05 | 2.21 |
| 34 | 8.49 | 0.14 | 3.24 | 8.04 | 0.15 | 9.12 |
| 35 | 7.9 | 0.11 | 12.5 | 7.57 | 0.03 | 26.7 |
| 36 | 6.75 | 0.2 | 178 | 6.23 | 0.06 | 590 |
| 37 | 8.47 | 0.06 | 3.38 | 8.54 | 0.04 | 2.92 |
| 38 | 6.81 | 0.14 | 156 | 6.28 | 0.07 | 520 |
| 39 | 8.04 | 0.06 | 9.18 | 7.8 | 0.05 | 16 |
| 40 | 8.5 | 0.08 | 3.17 | 8.12 | 0.04 | 7.64 |
| 41 | 8.05 | 0.12 | 8.83 | 7.53 | 0.04 | 29.6 |
| 42 | 6.99 | 0.11 | 104 | 6.4 | 0.06 | 403 |
| 43 | 7.53 | 0.06 | 29.4 | 7.18 | 0.05 | 65.5 |
| 44 | 7.44 | 0.08 | 36.1 | 7.03 | 0.04 | 92.9 |
| 45 | 8.3 | 0.03 | 5.07 | 8.39 | 0.04 | 4.05 |
| 46 | 8.35 | 0.06 | 4.5 | 8.4 | 0.05 | 3.98 |
| 47 | 8.3 | 0.11 | 5 | 8.58 | 0.03 | 2.64 |
| 48 | 8.74 | 0.1 | 1.84 | 9.03 | 0.05 | 0.942 |
| 49 | 8.5 | 0.04 | 3.18 | 8.18 | 0.03 | 6.68 |
| 50 | 8.11 | 0.07 | 7.78 | 8 | 0.04 | 10.1 |
| 51 | 8.62 | 0.05 | 2.38 | 8.19 | 0.05 | 6.47 |
| 52 | 8.33 | 0.08 | 4.73 | 8.31 | 0.05 | 4.91 |
| 53 | 6.95 | 0.08 | 111 | 6.6 | 0.05 | 252 |

*Compound 1 was run four times as a plate control and compound 10 was run in duplicate.

TABLE 2
Biochemical potency (pIC50) for selected SHP2 inhibitors on SHP2 D61G alone
(Non-Activated) and in presence of 0.5 μM NsCs peptide (Activated)

| | Potency of compounds for inhibition of human SHP2-FL D61G | | | | | |
|---|---|---|---|---|---|---|
| | SHP2 D61G | | | SHP2 D61G | | |
| | No Peptide Control | | | 0.5 μM NsCs peptide | | |
| | (Non-Activated) | | | (Activated) | | |
| | | Standard | IC₅₀ | | Standard | IC₅₀ |
| Compound | pIC₅₀ | Error^{†} | (nM)* | pIC₅₀ | Error^{†} | (nM)* |
| 1 | 8.73 | 0.07 | 1.85 | 6.73 | 0.08 | 185 |
| 1 | 8.61 | 0.05 | 2.45 | 6.66 | 0.05 | 219 |
| 1 | 8.8 | 0.06 | 1.6 | 6.7 | 0.04 | 198 |
| 1 | 8.74 | 0.12 | 1.81 | 6.72 | 0.06 | 191 |
| 2 | 7.25 | 0.04 | 55.7 | 5.15 | 0.17 | 7100 |
| 3 | 7.57 | 0.03 | 26.7 | 5.52 | 0.14 | 3050 |
| 4 | 7.39 | 0.05 | 40.5 | 5.27 | 0.15 | 5350 |
| 5 | 7.18 | 0.03 | 66.2 | 4.94 | 0.27 | 11500 |
| 6 | 7.43 | 0.06 | 37 | 5.7 | 0.1 | 2010 |
| 7 | 7.74 | 0.04 | 18.2 | 5.67 | 0.15 | 2120 |
| 8 | 8.18 | 0.04 | 6.65 | 6.27 | 0.07 | 542 |
| 9 | 6.68 | 0.03 | 208 | <5 | NA | >10000 |
| 10 | 7.77 | 0.09 | 16.8 | 6.2 | 0.07 | 638 |
| 10 | 7.75 | 0.06 | 17.7 | 6.15 | 0.12 | 701 |
| 11 | 7.36 | 0.08 | 43.3 | 5.79 | 0.16 | 1610 |
| 12 | 7.89 | 0.04 | 12.8 | 5.67 | 0.11 | 2150 |
| 13 | 8.48 | 0.05 | 3.32 | 6.54 | 0.05 | 287 |
| 14 | 7.34 | 0.04 | 45.6 | 5.1 | 0.27 | 7960 |
| 15 | 7.11 | 0.04 | 76.9 | 5.32 | 0.17 | 4810 |
| 16 | 8.28 | 0.03 | 5.25 | 6.18 | 0.11 | 659 |
| 17 | 6.7 | 0.04 | 198 | <5 | NA | >10000 |
| 18 | 6.5 | 0.06 | 319 | <5 | NA | >10000 |
| 19 | 5.92 | 0.06 | 1200 | <5 | NA | >10000 |
| 20 | 9.05 | 0.05 | 0.883 | 6.99 | 0.05 | 104 |
| 21 | 8.71 | 0.05 | 1.95 | 6.84 | 0.09 | 144.00 |
| 22 | 6.75 | 0.03 | 177 | <5 | NA | >10000 |
| 23 | 7.6 | 0.03 | 25.1 | 5.45 | 0.1 | 3530 |
| 24 | 8.65 | 0.06 | 2.24 | 6.76 | 0.11 | 173 |
| 25 | 7.24 | 0.04 | 57.9 | 5.32 | 0.25 | 4780 |
| 26 | 7.68 | 0.06 | 21 | 5.81 | 0.14 | 1550 |
| 27 | 8.08 | 0.06 | 8.41 | 6.53 | 0.09 | 294 |
| 28 | 8.28 | 0.06 | 5.25 | 6.34 | 0.04 | 462 |
| 29 | 8.52 | 0.04 | 3.04 | 6.49 | 0.05 | 322 |
| 30 | 8.17 | 0.03 | 6.71 | 6.13 | 0.06 | 738 |
| 31 | 6.53 | 0.05 | 298 | <5 | NA | >10000 |
| 32 | 8.63 | 0.05 | 2.33 | 7.02 | 0.05 | 94.6 |
| 33 | 8.55 | 0.05 | 2.79 | 6.99 | 0.05 | 104 |
| 34 | 8.25 | 0.03 | 5.61 | 6.14 | 0.09 | 723 |
| 35 | 7.66 | 0.03 | 21.9 | 5.68 | 0.05 | 2080 |
| 36 | 6.24 | 0.06 | 570 | <5 | NA | >10000 |
| 37 | 8.46 | 0.04 | 3.51 | 6.63 | 0.05 | 233 |
| 38 | 6.34 | 0.03 | 453 | 4.93 | 0.27 | 11800 |
| 39 | 7.77 | 0.05 | 16.9 | 5.81 | 0.06 | 1550 |
| 40 | 8.27 | 0.03 | 5.38 | 6.23 | 0.04 | 587 |
| 41 | 7.45 | 0.03 | 35.9 | 5.34 | 0.09 | 4540 |
| 42 | 6.4 | 0.06 | 395 | <5 | NA | >10000 |
| 43 | 7.14 | 0.05 | 73.1 | 5.3 | 0.11 | 5070 |
| 44 | 7.08 | 0.04 | 83.8 | 5.19 | 0.34 | 6490 |
| 45 | 8.26 | 0.04 | 5.52 | 6.64 | 0.03 | 229 |
| 46 | 8.37 | 0.05 | 4.32 | 6.78 | 0.03 | 166 |
| 47 | 8.44 | 0.05 | 3.65 | 6.78 | 0.04 | 167 |
| 48 | 9 | 0.04 | 0.993 | 7.14 | 0.05 | 72.8 |
| 49 | 8.16 | 0.03 | 6.9 | 6.12 | 0.06 | 753 |
| 50 | 8.09 | 0.05 | 8.15 | 5.9 | 0.1 | 1250 |
| 51 | 8.48 | 0.05 | 3.33 | 6.39 | 0.09 | 406 |
| 52 | 8.44 | 0.05 | 3.66 | 6.62 | 0.03 | 238 |
| 53 | 6.67 | 0.05 | 213 | <5 | NA | >10000 |

TABLE 3
Biochemical potency (pIC50) for selected SHP2 inhibitors on SHP2 E76K alone
(Non-Activated) and in presence of 0.5 μM NsCs peptide (Activated)

| | Potency of compounds for inhibition of human SHP2-FL E76K | | | | | |
|---|---|---|---|---|---|---|
| | SHP2 E76K | | | SHP2 E76K | | |
| | No Peptide Control | | | 0.5 μM NsCs peptide | | |
| | (Non-Activated) | | | (Activated) | | |
| | | Standard | IC₅₀ | | Standard | IC₅₀ |
| Compound | pIC₅₀ | Error^{†} | (nM)* | pIC₅₀ | Error^{†} | (nM)* |
| 1 | 7.27 | 0.09 | 54 | <5 | NA | >10000 |
| 1 | 7.23 | 0.07 | 58.3 | 4.88 | 0.34 | 13100 |
| 1 | 7.3 | 0.08 | 50.6 | 4.98 | 0.29 | 10400 |
| 1 | 7.27 | 0.08 | 54.2 | <5 | NA | >10000 |
| 2 | 5.68 | 0.16 | 2090 | <5 | NA | >10000 |
| 3 | 6 | 0.05 | 991 | <5 | NA | >10000 |
| 4 | 5.8 | 0.14 | 1580 | <5 | NA | >10000 |
| 5 | 5.41 | 0.12 | 3870 | <5 | NA | >10000 |
| 6 | 6.41 | 0.04 | 393 | <5 | NA | >10000 |
| 7 | 6.45 | 0.04 | 358 | <5 | NA | >10000 |
| 8 | 6.84 | 0.03 | 145 | <5 | NA | >10000 |
| 9 | 5.24 | 0.11 | 5750 | <5 | NA | >10000 |
| 10 | 6.87 | 0.05 | 135 | <5 | NA | >10000 |
| 10 | 6.85 | 0.04 | 141 | <5 | NA | >10000 |
| 11 | 6.36 | 0.1 | 433 | <5 | NA | >10000 |
| 12 | 6.33 | 0.04 | 468 | <5 | NA | >10000 |
| 13 | 7.18 | 0.09 | 66.7 | <5 | NA | >10000 |
| 14 | 5.51 | 0.26 | 3120 | <5 | NA | >10000 |
| 15 | 5.77 | 0.21 | 1710 | <5 | NA | >10000 |
| 16 | 6.79 | 0.04 | 162 | <5 | NA | >10000 |
| 17 | 5.48 | 0.13 | 3320 | <5 | NA | >10000 |
| 18 | 5.16 | 0.14 | 6950 | <5 | NA | >10000 |
| 19 | 4.96 | 0.34 | 10900 | <5 | NA | >10000 |
| 20 | 7.54 | 0.06 | 29 | 5 | 0.17 | 9930 |
| 21 | 7.60 | 0.09 | 25.00 | <5 | NA | >10000 |
| 22 | 5.47 | 0.11 | 3400 | <5 | NA | >10000 |
| 23 | 6.15 | 0.06 | 714 | <5 | NA | >10000 |
| 24 | 7.15 | 0.07 | 70.6 | <6 | NA | >10001 |
| 25 | 5.95 | 0.11 | 1120 | <5 | NA | >10000 |
| 26 | 6.41 | 0.11 | 393 | <5 | NA | >10000 |
| 27 | 7.16 | 0.04 | 69.8 | <5 | NA | >10000 |
| 28 | 7.1 | 0.05 | 78.7 | <5 | NA | >10000 |
| 29 | 7.18 | 0.05 | 65.6 | <5 | NA | >10000 |
| 30 | 6.8 | 0.06 | 160 | <5 | NA | >10000 |
| 31 | 5.34 | 0.22 | 4610 | <5 | NA | >10000 |
| 32 | 7.69 | 0.05 | 20.3 | 4.94 | 0.22 | 11500 |
| 33 | 7.6 | 0.06 | 25.2 | 4.96 | 0.37 | 11100 |
| 34 | 6.98 | 0.05 | 105 | <5 | NA | >10000 |
| 35 | 6.26 | 0.03 | 553 | <5 | NA | >10000 |
| 36 | 4.93 | 0.45 | 11700 | <5 | NA | >10000 |
| 37 | 7.45 | 0.04 | 35.9 | 4.96 | 0.19 | 11000 |
| 38 | 5.33 | 0.1 | 4660 | <5 | NA | >10000 |
| 39 | 6.45 | 0.06 | 352 | <5 | NA | >10000 |
| 40 | 6.84 | 0.04 | 143 | <5 | NA | >10000 |
| 41 | 6.05 | 0.06 | 895 | <5 | NA | >10000 |
| 42 | 5.29 | 0.13 | 5090 | <5 | NA | >10000 |
| 43 | 6.14 | 0.05 | 718 | <5 | NA | >10000 |
| 44 | 6.11 | 0.05 | 783 | <5 | NA | >10000 |
| 45 | 7.3 | 0.05 | 50.2 | 4.85 | 0.32 | 14100 |
| 46 | 7.41 | 0.03 | 38.9 | 5.25 | 0.17 | 5690 |
| 47 | 7.44 | 0.03 | 36.1 | 5.47 | 0.11 | 3400 |
| 48 | 7.94 | 0.06 | 11.4 | 5.24 | 0.14 | 5790 |
| 49 | 6.79 | 0.03 | 163 | <5 | NA | >10000 |
| 50 | 6.57 | 0.06 | 267 | <5 | NA | >10000 |
| 51 | 7.25 | 0.02 | 56.9 | <6 | NA | >10001 |
| 52 | 7.37 | 0.04 | 43 | 5.18 | 0.17 | 6680 |
| 53 | 5.42 | 0.08 | 3850 | <5 | NA | >10000 |

TABLE 4
Biochemical potency (pIC50) for selected SHP2 inhibitors on SHP2 S189A alone
(Non-Activated) and in presence of 0.5 μM NsCs peptide (Activated)

| | Potency of compounds for inhibition of human SHP2-FL S189A | | | | | |
|---|---|---|---|---|---|---|
| | SHP2 S189A | | | SHP2 S189A | | |
| | No Peptide Control | | | 0.5 μM NsCs peptide | | |
| | (Non-Activated) | | | (Activated) | | |
| | | Standard | IC₅₀ | | Standard | IC₅₀ |
| Compound | pIC₅₀ | Error^{†} | (nM)* | pIC₅₀ | Error^{†} | (nM)* |
| 1 | 8.52 | 0.15 | 3.01 | 8.47 | 0.07 | 3.38 |
| 1 | 8.48 | 0.11 | 3.35 | 8.43 | 0.05 | 3.76 |
| 1 | 8.56 | 0.14 | 2.75 | 8.52 | 0.04 | 2.99 |
| 1 | 8.51 | 0.13 | 3.11 | 8.42 | 0.07 | 3.79 |
| 2 | 7.49 | 0.13 | 32.5 | 7.04 | 0.06 | 90.8 |
| 3 | 7.79 | 0.17 | 16.4 | 7.41 | 0.07 | 39.4 |
| 4 | 7.58 | 0.11 | 26.3 | 7.23 | 0.05 | 59.4 |
| 5 | 7.54 | 0.11 | 28.9 | 6.96 | 0.05 | 109 |
| 6 | 7.55 | 0.12 | 28.2 | 7.3 | 0.08 | 50.1 |
| 7 | 7.94 | 0.08 | 11.6 | 7.56 | 0.03 | 27.9 |
| 8 | 8.13 | 0.13 | 7.45 | 7.98 | 0.05 | 10.5 |
| 9 | 6.93 | 0.08 | 117 | 6.55 | 0.02 | 283 |
| 10 | 7.73 | 0.11 | 18.6 | 7.69 | 0.06 | 20.4 |
| 10 | 7.72 | 0.09 | 19 | 7.69 | 0.05 | 20.4 |
| 11 | 7.45 | 0.23 | 35.6 | 7.28 | 0.08 | 52.6 |
| 12 | 8.02 | 0.13 | 9.57 | 7.65 | 0.05 | 22.5 |
| 13 | 8.32 | 0.12 | 4.82 | 8.19 | 0.05 | 6.4 |
| 14 | 7.67 | 0.11 | 21.3 | 7.24 | 0.05 | 57.1 |
| 15 | 7.23 | 0.1 | 59.3 | 7.01 | 0.03 | 97.7 |
| 16 | 8.11 | 0.12 | 7.73 | 8.04 | 0.03 | 9.14 |
| 17 | 6.99 | 0.07 | 102 | 6.58 | 0.02 | 262 |
| 18 | 6.64 | 0.1 | 227 | 6.38 | 0.05 | 419 |
| 19 | 6.19 | 0.22 | 650 | 5.79 | 0.07 | 1610 |
| 20 | 8.91 | 0.09 | 1.24 | 8.82 | 0.04 | 1.51 |
| 21 | 8.50 | 0.19 | 3.16 | 8.60 | 0.06 | 2.52 |
| 22 | 6.8 | 0.12 | 160 | 6.61 | 0.07 | 245 |
| 23 | 7.98 | 0.18 | 10.4 | 7.4 | 0.05 | 39.7 |
| 24 | 8.57 | 0.13 | 2.67 | 8.37 | 0.06 | 4.31 |
| 25 | 7.38 | 0.12 | 41.6 | 7.12 | 0.05 | 76.2 |
| 26 | 7.69 | 0.13 | 20.5 | 7.5 | 0.08 | 31.7 |
| 27 | 7.94 | 0.12 | 11.5 | 7.92 | 0.06 | 11.9 |
| 28 | 8.24 | 0.08 | 5.73 | 8.05 | 0.04 | 8.89 |
| 29 | 8.3 | 0.07 | 4.97 | 8.23 | 0.04 | 5.87 |
| 30 | 8.25 | 0.08 | 5.58 | 7.98 | 0.03 | 10.5 |
| 31 | 6.69 | 0.08 | 203 | 6.43 | 0.05 | 376 |
| 32 | 8.34 | 0.09 | 4.53 | 8.47 | 0.04 | 3.39 |
| 33 | 8.33 | 0.1 | 4.7 | 8.52 | 0.04 | 2.99 |
| 34 | 8.18 | 0.09 | 6.64 | 7.99 | 0.04 | 10.3 |
| 35 | 7.66 | 0.07 | 21.7 | 7.43 | 0.02 | 37.2 |
| 36 | 6.67 | 0.11 | 216 | 5.99 | 0.06 | 1030 |
| 37 | 8.3 | 0.11 | 4.99 | 8.29 | 0.02 | 5.13 |
| 38 | 6.31 | 0.12 | 486 | 6.22 | 0.05 | 604 |
| 39 | 7.77 | 0.13 | 16.9 | 7.62 | 0.05 | 23.8 |
| 40 | 8.21 | 0.08 | 6.15 | 8.02 | 0.04 | 9.55 |
| 41 | 7.59 | 0.15 | 25.6 | 7.32 | 0.03 | 47.8 |
| 42 | 6.55 | 0.1 | 284 | 6.22 | 0.06 | 610 |
| 43 | 7.2 | 0.09 | 62.7 | 7.03 | 0.04 | 92.7 |
| 44 | 7.01 | 0.09 | 97.9 | 6.86 | 0.03 | 140 |
| 45 | 8.05 | 0.08 | 8.83 | 8.2 | 0.03 | 6.31 |
| 46 | 8.13 | 0.07 | 7.41 | 8.28 | 0.04 | 5.31 |
| 47 | 8.26 | 0.08 | 5.51 | 8.38 | 0.04 | 4.13 |
| 48 | 8.61 | 0.12 | 2.48 | 8.79 | 0.03 | 1.62 |
| 49 | 8.17 | 0.06 | 6.71 | 8 | 0.05 | 9.95 |
| 50 | 7.92 | 0.08 | 12 | 7.85 | 0.04 | 14.1 |
| 51 | 8.19 | 0.07 | 6.41 | 8.21 | 0.03 | 6.24 |
| 52 | 8.17 | 0.13 | 6.79 | 8.29 | 0.04 | 5.11 |
| 53 | 6.74 | 0.1 | 184 | 6.35 | 0.06 | 447 |

TABLE 5
Biochemical potency (pIC50) for selected SHP2 inhibitors on SHP2 L262R alone
(Non-Activated) and in presence of 0.5 μM NsCs peptide (Activated)

| | Potency of compounds for inhibition of human SHP2-FL L262R | | | | | |
|---|---|---|---|---|---|---|
| | SHP2 L262R | | | SHP2 L262R | | |
| | No Peptide Control | | | 0.5 μM NsCs peptide | | |
| | (Non-Activated) | | | (Activated) | | |
| | | Standard | IC₅₀ | | Standard | IC₅₀ |
| Compound | pIC₅₀ | Error^{†} | (nM)* | pIC₅₀ | Error^{†} | (nM)* |
| 1 | 8.55 | 0.05 | 2.83 | 7.1 | 0.17 | 79.4 |
| 1 | 8.47 | 0.05 | 3.37 | 7.21 | 0.1 | 62.2 |
| 1 | 8.56 | 0.05 | 2.75 | 7.16 | 0.09 | 69.2 |
| 1 | 8.47 | 0.06 | 3.43 | 7.13 | 0.05 | 74.6 |
| 2 | 7.12 | 0.04 | 75.9 | 5.7 | 0.2 | 2000 |
| 3 | 7.43 | 0.02 | 37 | 6.03 | 0.15 | 927 |
| 4 | 7.21 | 0.05 | 61.1 | 5.86 | 0.14 | 1390 |
| 5 | 6.9 | 0.04 | 127 | 5.29 | 0.4 | 5180 |
| 6 | 7.36 | 0.06 | 43.4 | 6.34 | 0.11 | 457 |
| 7 | 7.77 | 0.03 | 17.1 | 6.41 | 0.08 | 385 |
| 8 | 8.04 | 0.04 | 9.12 | 6.87 | 0.09 | 136 |
| 9 | 6.58 | 0.03 | 262 | <5 | NA | >10000 |
| 10 | 7.73 | 0.06 | 18.6 | 6.65 | 0.14 | 224 |
| 10 | 7.74 | 0.05 | 18.2 | 6.83 | 0.07 | 148 |
| 11 | 7.34 | 0.08 | 45.5 | 6.21 | 0.23 | 621 |
| 12 | 7.67 | 0.03 | 21.6 | 6.35 | 0.05 | 448 |
| 13 | 8.41 | 0.03 | 3.85 | 6.98 | 0.06 | 105 |
| 14 | 7.16 | 0.04 | 68.7 | 5.57 | 0.26 | 2670 |
| 15 | 7.08 | 0.05 | 83.4 | 5.71 | 0.24 | 1940 |
| 16 | 7.98 | 0.07 | 10.5 | 6.6 | 0.14 | 254 |
| 17 | 6.83 | 0.02 | 147 | 5.25 | 0.07 | 5640 |
| 18 | 6.53 | 0.04 | 296 | <5 | NA | >10000 |
| 19 | 6.07 | 0.05 | 847 | <5 | NA | >10000 |
| 20 | 8.7 | 0.02 | 1.98 | 7.46 | 0.03 | 34.5 |
| 21 | 8.62 | 0.05 | 2.39 | 7.28 | 0.11 | 52.20 |
| 22 | 6.85 | 0.03 | 141 | 5.3 | 0.22 | 5070 |
| 23 | 7.47 | 0.03 | 34.3 | 6.02 | 0.06 | 962 |
| 24 | 8.41 | 0.02 | 3.89 | 7.03 | 0.1 | 93.1 |
| 25 | 7.21 | 0.05 | 62.4 | 5.77 | 0.28 | 1710 |
| 26 | 7.6 | 0.04 | 25.4 | 6.41 | 0.07 | 391 |
| 27 | 8.01 | 0.05 | 9.77 | 7.1 | 0.07 | 79.4 |
| 28 | 8.21 | 0.03 | 6.15 | 6.79 | 0.04 | 161 |
| 29 | 8.29 | 0.02 | 5.14 | 6.82 | 0.08 | 152 |
| 30 | 8.02 | 0.03 | 9.62 | 6.66 | 0.04 | 219 |
| 31 | 6.48 | 0.05 | 333 | <5 | NA | >10000 |
| 32 | 8.54 | 0.05 | 2.91 | 7.55 | 0.09 | 28.1 |
| 33 | 8.51 | 0.06 | 3.13 | 7.6 | 0.07 | 25.1 |
| 34 | 8.16 | 0.02 | 6.92 | 6.91 | 0.05 | 123 |
| 35 | 7.5 | 0.03 | 31.5 | 6.05 | 0.11 | 897 |
| 36 | 6.04 | 0.06 | 920 | <5 | NA | >10000 |
| 37 | 8.51 | 0.03 | 3.11 | 7.15 | 0.07 | 71.6 |
| 38 | 6.27 | 0.05 | 535 | <5 | NA | >10000 |
| 39 | 7.71 | 0.04 | 19.7 | 6.37 | 0.13 | 423 |
| 40 | 8.12 | 0.02 | 7.53 | 6.67 | 0.14 | 215 |
| 41 | 7.41 | 0.03 | 39.1 | 5.99 | 0.09 | 1030 |
| 42 | 6.5 | 0.05 | 320 | <5 | NA | >10000 |
| 43 | 7.2 | 0.05 | 63.5 | 6.09 | 0.05 | 813 |
| 44 | 7.05 | 0.03 | 89.1 | 5.58 | 0.18 | 2650 |
| 45 | 8.26 | 0.04 | 5.45 | 7.25 | 0.05 | 56.5 |
| 46 | 8.36 | 0.06 | 4.38 | 7.6 | 0.03 | 25.2 |
| 47 | 8.41 | 0.05 | 3.92 | 7.84 | 0.08 | 14.5 |
| 48 | 8.9 | 0.06 | 1.25 | 7.67 | 0.04 | 21.5 |
| 49 | 8.01 | 0.02 | 9.68 | 6.69 | 0.08 | 204 |
| 50 | 7.96 | 0.04 | 10.9 | 6.39 | 0.19 | 409 |
| 51 | 8.35 | 0.02 | 4.49 | 7.1 | 0.05 | 79.6 |
| 52 | 8.4 | 0.05 | 3.95 | 7.32 | 0.08 | 47.5 |
| 53 | 6.83 | 0.04 | 148 | <5 | NA | >10000 |

TABLE 6
Biochemical potency (pIC50) for selected SHP2 inhibitors on SHP2 F285S alone
(Non-Activated) and in presence of 0.5 μM NsCs peptide (Activated)

| | Potency of compounds for inhibition of human SHP2-FL F285S | | | | | |
|---|---|---|---|---|---|---|
| | SHP2 F285S | | | SHP2 F285S | | |
| | No Peptide Control | | | 0.5 μM NsCs peptide | | |
| | (Non-Activated) | | | (Activated) | | |
| | | Standard | IC₅₀ | | Standard | IC₅₀ |
| Compound | pIC₅₀ | Error^{†} | (nM)* | pIC₅₀ | Error^{†} | (nM)* |
| 1 | 8.81 | 0.08 | 1.57 | 8.43 | 0.11 | 3.76 |
| 1 | 8.81 | 0.12 | 1.55 | 8.04 | 0.23 | 9.08 |
| 1 | 8.98 | 0.09 | 1.04 | 8.08 | 0.2 | 8.38 |
| 1 | 8.92 | 0.09 | 1.19 | 7.82 | 0.21 | 15.1 |
| 2 | 7.76 | 0.1 | 17.6 | 6.59 | 0.36 | 258 |
| 3 | 8.03 | 0.08 | 9.25 | 7.38 | 0.19 | 42.2 |
| 4 | 7.56 | 0.1 | 27.3 | 6.73 | 0.28 | 185 |
| 5 | 7.49 | 0.11 | 32.7 | 6.68 | 0.16 | 209 |
| 6 | 7.4 | 0.09 | 40.2 | 6.79 | 0.21 | 162 |
| 7 | 8.46 | 0.09 | 3.47 | 7.24 | 0.19 | 58.1 |
| 8 | 7.99 | 0.07 | 10.2 | 7.22 | 0.14 | 60.1 |
| 9 | 7.04 | 0.15 | 91 | 6.33 | 0.24 | 468 |
| 10 | 7.75 | 0.08 | 17.8 | 7.36 | 0.07 | 44.1 |
| 10 | 7.77 | 0.08 | 17 | 7.4 | 0.09 | 40 |
| 11 | 7.59 | 0.08 | 25.5 | 7.1 | 0.17 | 80.2 |
| 12 | 8.41 | 0.12 | 3.92 | 7.36 | 0.11 | 43.4 |
| 13 | 8.87 | 0.08 | 1.36 | 7.59 | 0.15 | 25.5 |
| 14 | 7.8 | 0.12 | 16 | 6.76 | 0.21 | 172 |
| 15 | 7.25 | 0.17 | 56 | 6.11 | 0.44 | 778 |
| 16 | 8.28 | 0.17 | 5.3 | 7.48 | 0.17 | 33.4 |
| 17 | 7.4 | 0.13 | 40.2 | 6.42 | 0.17 | 378 |
| 18 | 7.14 | 0.13 | 71.8 | 6.59 | 0.2 | 260 |
| 19 | 6.88 | 0.12 | 133 | 6.2 | 0.16 | 632 |
| 20 | 8.99 | 0.12 | 1.02 | 8.43 | 0.2 | 3.7 |
| 21 | 8.69 | 0.10 | 2.05 | 7.72 | 0.13 | 19.00 |
| 22 | 7.46 | 0.12 | 35.1 | 6.72 | 0.17 | 190 |
| 23 | 8.05 | 0.12 | 8.95 | 7 | 0.16 | 101 |
| 24 | 8.97 | 0.08 | 1.08 | 8.07 | 0.14 | 8.47 |
| 25 | 7.39 | 0.14 | 40.5 | 6.52 | 0.15 | 305 |
| 26 | 7.72 | 0.09 | 19 | 7.28 | 0.27 | 53 |
| 27 | 7.86 | 0.09 | 13.8 | 7.46 | 0.26 | 34.8 |
| 28 | 8.66 | 0.11 | 2.21 | 7.48 | 0.11 | 33 |
| 29 | 8.55 | 0.08 | 2.82 | 7.79 | 0.12 | 16.1 |
| 30 | 8.64 | 0.11 | 2.32 | 7.92 | 0.16 | 12.1 |
| 31 | 6.96 | 0.15 | 111 | 5.97 | 0.4 | 1080 |
| 32 | 8.28 | 0.07 | 5.31 | 7.61 | 0.12 | 24.4 |
| 33 | 8.7 | 0.06 | 2 | 7.89 | 0.11 | 12.9 |
| 34 | 8.49 | 0.11 | 3.23 | 7.89 | 0.16 | 12.9 |
| 35 | 8.15 | 0.18 | 7.05 | 6.9 | 0.15 | 125 |
| 36 | 6.92 | 0.16 | 120 | <5 | NA | >10000 |
| 37 | 8.7 | 0.11 | 2 | 7.95 | 0.11 | 11.3 |
| 38 | 7.03 | 0.11 | 92.9 | 6.83 | 0.23 | 147 |
| 39 | 8.23 | 0.1 | 5.96 | 7.71 | 0.19 | 19.6 |
| 40 | 8.52 | 0.15 | 3.03 | 7.58 | 0.17 | 26.5 |
| 41 | 8.2 | 0.13 | 6.25 | 6.96 | 0.1 | 110 |
| 42 | 7.21 | 0.12 | 61.8 | 6.27 | 0.14 | 536 |
| 43 | 7.28 | 0.12 | 52.8 | 6.9 | 0.13 | 125 |
| 44 | 7.27 | 0.1 | 53.3 | 6.48 | 0.13 | 329 |
| 45 | 8.3 | 0.08 | 4.97 | 7.83 | 0.09 | 14.7 |
| 46 | 8.17 | 0.14 | 6.78 | 7.58 | 0.09 | 26.1 |
| 47 | 8.5 | 0.11 | 3.18 | 7.58 | 0.09 | 26.2 |
| 48 | 8.68 | 0.08 | 2.07 | 8.12 | 0.1 | 7.55 |
| 49 | 8.32 | 0.09 | 4.78 | 7.36 | 0.11 | 43.5 |
| 50 | 8.58 | 0.08 | 2.64 | 7.19 | 0.14 | 64.1 |
| 51 | 8.56 | 0.12 | 2.75 | 7.74 | 0.1 | 18.1 |
| 52 | 8.49 | 0.06 | 3.21 | 7.35 | 0.12 | 44.3 |
| 53 | 7.3 | 0.12 | 49.9 | 5.72 | 0.35 | 1900 |

TABLE 7
Biochemical potency (pIC50) for selected SHP2 inhibitors on SHP2 P491S alone
(Non-Activated) and in presence of 0.5 μM NsCs peptide (Activated)

| | Potency of compounds for inhibition of human SHP2-FL P491S | | | | | |
|---|---|---|---|---|---|---|
| | SHP2 P491S | | | SHP2 P491S | | |
| | No Peptide Control | | | 0.5 μM NsCs peptide | | |
| | (Non-Activated) | | | (Activated) | | |
| | | Standard | IC₅₀ | | Standard | IC₅₀ |
| Compound | pIC₅₀ | Error^{†} | (nM)* | pIC₅₀ | Error^{†} | (nM)* |
| 1 | 6.59 | 0.06 | 258 | <5 | NA | >10000 |
| 1 | 6.56 | 0.05 | 274 | <5 | NA | >10000 |
| 1 | 6.6 | 0.05 | 253 | 4.97 | 0.16 | 10600 |
| 1 | 6.55 | 0.05 | 282 | 5.12 | 0.09 | 7670 |
| 2 | 5.88 | 0.07 | 1330 | <5 | NA | >10000 |
| 3 | 6.06 | 0.03 | 879 | <5 | NA | >10000 |
| 4 | 5.85 | 0.09 | 1410 | <5 | NA | >10000 |
| 5 | 5.53 | 0.08 | 2990 | <5 | NA | >10000 |
| 6 | 5.85 | 0.07 | 1420 | <5 | NA | >10000 |
| 7 | 5.87 | 0.06 | 1360 | <5 | NA | >10000 |
| 8 | 6.14 | 0.05 | 721 | <5 | NA | >10000 |
| 9 | 5.39 | 0.07 | 4070 | <5 | NA | >10000 |
| 10 | 6.63 | 0.05 | 235 | 5.42 | 0.12 | 3820 |
| 10 | 6.7 | 0.03 | 200 | 5.31 | 0.08 | 4940 |
| 11 | 5.85 | 0.11 | 1430 | <5 | NA | >10000 |
| 12 | 5.73 | 0.06 | 1880 | <5 | NA | >10000 |
| 13 | 6.86 | 0.05 | 139 | 5.41 | 0.12 | 3920 |
| 14 | 5.45 | 0.11 | 3550 | <5 | NA | >10000 |
| 15 | 5.34 | 0.12 | 4600 | <5 | NA | >10000 |
| 16 | 6.16 | 0.03 | 689 | <5 | NA | >10000 |
| 17 | <5 | NA | >10000 | <5 | NA | >10000 |
| 18 | <5 | NA | >10000 | <5 | NA | >10000 |
| 19 | <5 | NA | >10000 | <5 | NA | >10000 |
| 20 | 6.79 | 0.03 | 161 | 5.46 | 0.07 | 3440 |
| 21 | 6.67 | 0.06 | 214.00 | 5.04 | 0.19 | 9230.00 |
| 22 | <5 | NA | >10000 | <5 | NA | >10000 |
| 23 | 5.6 | 0.06 | 2490 | <5 | NA | >10000 |
| 24 | 6.47 | 0.05 | 340 | 5.2 | 0.18 | 6310 |
| 25 | 5.35 | 0.17 | 4470 | <5 | NA | >10000 |
| 26 | 5.87 | 0.1 | 1360 | <5 | NA | >10000 |
| 27 | 6.9 | 0.05 | 126 | 5.54 | 0.09 | 2920 |
| 28 | 6.22 | 0.03 | 600 | <5 | NA | >10000 |
| 29 | 6.49 | 0.04 | 325 | 5.05 | 0.24 | 8910 |
| 30 | 6.01 | 0.05 | 986 | <5 | NA | >10000 |
| 31 | <5 | NA | >10000 | <5 | NA | >10000 |
| 32 | 7.42 | 0.04 | 38.2 | 6.18 | 0.05 | 658 |
| 33 | 7.47 | 0.05 | 34 | 6.13 | 0.08 | 741 |
| 34 | 6.65 | 0.02 | 226 | 5.24 | 0.15 | 5740 |
| 35 | 5.67 | 0.04 | 2150 | <5 | NA | >10000 |
| 36 | <5 | NA | >10000 | <5 | NA | >10000 |
| 37 | 7.05 | 0.02 | 89.5 | 5.54 | 0.15 | 2910 |
| 38 | <5 | NA | >10000 | <5 | NA | >10000 |
| 39 | 6.41 | 0.06 | 387 | <5 | NA | >10000 |
| 40 | 5.96 | 0.03 | 1100 | <5 | NA | >10000 |
| 41 | 5.28 | 0.1 | 5260 | <5 | NA | >10000 |
| 42 | 5.1 | 0.18 | 7960 | <5 | NA | >10000 |
| 43 | 5.86 | 0.04 | 1400 | <5 | NA | >10000 |
| 44 | 5.23 | 0.12 | 5930 | <5 | NA | >10000 |
| 45 | 7.19 | 0.03 | 64.9 | 5.75 | 0.16 | 1790 |
| 46 | 7.57 | 0.02 | 27.2 | 6.31 | 0.03 | 494 |
| 47 | 7.62 | 0.03 | 24 | 6.39 | 0.07 | 410 |
| 48 | 7.59 | 0.04 | 25.5 | 6.21 | 0.1 | 622 |
| 49 | 6.11 | 0.03 | 769 | <5 | NA | >10000 |
| 50 | 5.78 | 0.07 | 1640 | <5 | NA | >10000 |
| 51 | 7.09 | 0.02 | 81.3 | 5.74 | 0.06 | 1820 |
| 52 | 7.33 | 0.04 | 47.2 | 6.12 | 0.07 | 757 |
| 53 | <5 | NA | >10000 | <5 | NA | >10000 |

TABLE 8
Biochemical potency (pIC50) for selected SHP2 inhibitors on SHP2 S502P alone
(Non-Activated) and in presence of 0.5 μM NsCs peptide (Activated)

| | Potency of compounds for inhibition of human SHP2-FL S502P | | | | | |
|---|---|---|---|---|---|---|
| | SHP2 S502P | | | SHP2 S502P | | |
| | No Peptide Control | | | 0.5 μM NsCs peptide | | |
| | (Non-Activated) | | | (Activated) | | |
| | | Standard | IC₅₀ | | Standard | IC₅₀ |
| Compound | pIC₅₀ | Error^{†} | (nM)* | pIC₅₀ | Error^{†} | (nM)* |
| 1 | 6.63 | 0.07 | 237 | <5 | NA | >10000 |
| 1 | 6.64 | 0.1 | 227 | <5 | NA | >10000 |
| 1 | 6.66 | 0.11 | 221 | <5 | NA | >10000 |
| 1 | 6.51 | 0.06 | 308 | <5 | NA | >10000 |
| 2 | <5 | NA | >10000 | <5 | NA | >10000 |
| 3 | 5.89 | 0.12 | 1300 | <5 | NA | >10000 |
| 4 | 5.84 | 0.18 | 1440 | <5 | NA | >10000 |
| 5 | <5 | NA | >10000 | <5 | NA | >10000 |
| 6 | 5.68 | 0.13 | 2070 | <5 | NA | >10000 |
| 7 | 6.04 | 0.09 | 918 | <5 | NA | >10000 |
| 8 | 6.21 | 0.08 | 614 | <5 | NA | >10000 |
| 9 | <5 | NA | >10000 | <5 | NA | >10000 |
| 10 | 6.07 | 0.07 | 861 | <5 | NA | >10000 |
| 10 | 6.06 | 0.13 | 871 | <5 | NA | >10000 |
| 11 | 5.72 | 0.1 | 1930 | <5 | NA | >10000 |
| 12 | 5.98 | 0.15 | 1050 | <5 | NA | >10000 |
| 13 | 6.62 | 0.06 | 239 | <5 | NA | >10000 |
| 14 | <5 | NA | >10000 | <5 | NA | >10000 |
| 15 | 5.59 | 0.2 | 2580 | <5 | NA | >10000 |
| 16 | 6.25 | 0.14 | 562 | <5 | NA | >10000 |
| 17 | <5 | NA | >10000 | <5 | NA | >10000 |
| 18 | <5 | NA | >10000 | <5 | NA | >10000 |
| 19 | <5 | NA | >10000 | <5 | NA | >10000 |
| 20 | 6.78 | 0.1 | 166 | <5 | NA | >10000 |
| 21 | 6.83 | 0.06 | 148.00 | <5 | NA | >10000 |
| 22 | 5.92 | 0.21 | 1210 | <5 | NA | >10000 |
| 23 | 5.99 | 0.13 | 1020 | <5 | NA | >10000 |
| 24 | 6.57 | 0.07 | 269 | <5 | NA | >10000 |
| 25 | 5.5 | 0.15 | 3160 | <5 | NA | >10000 |
| 26 | 5.99 | 0.1 | 1040 | <5 | NA | >10000 |
| 27 | 6.32 | 0.07 | 474 | <5 | NA | >10000 |
| 28 | 6.38 | 0.1 | 420 | <5 | NA | >10000 |
| 29 | 6.38 | 0.05 | 417 | <5 | NA | >10000 |
| 30 | 6.22 | 0.07 | 604 | <5 | NA | >10000 |
| 31 | <5 | NA | >10000 | <5 | NA | >10000 |
| 32 | 6.93 | 0.06 | 117 | <5 | NA | >10000 |
| 33 | 7.07 | 0.07 | 84.5 | <5 | NA | >10000 |
| 34 | 6.38 | 0.17 | 415 | <5 | NA | >10000 |
| 35 | 5.81 | 0.27 | 1550 | <5 | NA | >10000 |
| 36 | <5 | NA | >10000 | <5 | NA | >10000 |
| 37 | 6.84 | 0.08 | 146 | <5 | NA | >10000 |
| 38 | <5 | NA | >10000 | <5 | NA | >10000 |
| 39 | 6.02 | 0.12 | 946 | <5 | NA | >10000 |
| 40 | 6.31 | 0.15 | 495 | <5 | NA | >10000 |
| 41 | 6.04 | 0.17 | 923 | <5 | NA | >10000 |
| 42 | <5 | NA | >10000 | <5 | NA | >10000 |
| 43 | 5.56 | 0.17 | 2760 | <5 | NA | >10000 |
| 44 | <5 | NA | >10000 | <5 | NA | >10000 |
| 45 | 6.79 | 0.05 | 164 | <5 | NA | >10000 |
| 46 | 6.83 | 0.12 | 147 | <5 | NA | >10000 |
| 47 | 6.93 | 0.07 | 119 | <5 | NA | >10000 |
| 48 | 6.97 | 0.07 | 106 | <5 | NA | >10000 |
| 49 | 6.1 | 0.13 | 802 | <5 | NA | >10000 |
| 50 | 6.17 | 0.07 | 676 | <5 | NA | >10000 |
| 51 | 6.63 | 0.14 | 237 | <5 | NA | >10000 |
| 52 | 6.7 | 0.07 | 202 | <5 | NA | >10000 |
| 53 | <5 | NA | >10000 | <5 | NA | >10000 |

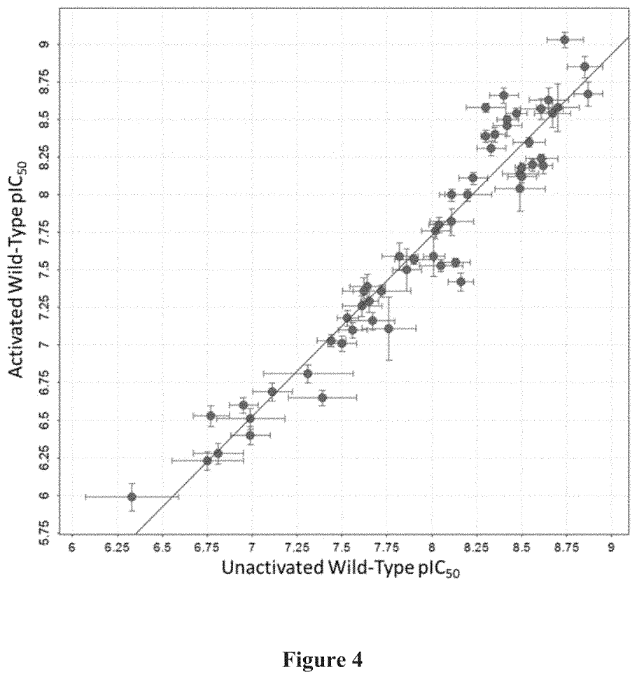

All 53 allosteric inhibitors of SHP2 tested inhibit wildtype and mutant SHP2s at pIC₅₀ values between 6 and 9. For each mutant, the trend in potency for mutant vs. wild-type can be approximated by a straight line, suggesting that the relative potencies of all compounds in this set are affected similarly by mutation. The activating peptide NsCs does not substantially increase or decrease the pIC₅₀ values for the tested compounds, as there were only negligible shift in potency for inhibition of wildtype SHP2 (FIG. 4).

In the absence of activating peptide, all mutant SHP2s tested are inhibited by the 53 allosteric inhibitors tested but inhibition of some mutants occurs only at higher inhibitor concentration than for wild-type SHP2. F285S, L262R, D61G, and S189A had very little effect on compound IC₅₀ values for non-activated SHP2. In contrast, E76K, P491S, and S502P produced a substantial (˜100-fold) reduction in potency for inhibition of the unactivated state, relative to wild-type SHP2.

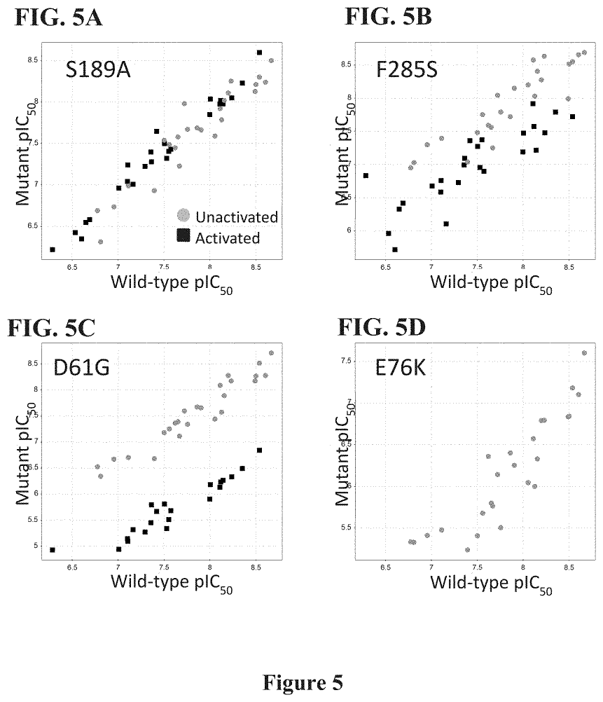

In the presence of the activating peptide, mutations show a peptide-driven shift in inhibitor potency of varying magnitude. The peptide shifted IC₅₀ values 3-fold or less for S189A and F285S. The peptide shifted IC₅₀ values 10- to 30-fold for D61G and L262R. The peptide shifted IC₅₀ values 100- to 1000-fold for E76K and P491S. S502P exhibited a peptide-driven potency shift of at least 100-fold, but the exact shift could not be determined because no inhibitory activity was detected for any compound (up to the highest test concentration of 10 μM) in the presence of activating peptide. The shift for S189A, F285S, D61G, and E76K are shown in FIG. 5.

Collectively, these biochemical data suggest that the SHP2 mutants profiled in this study are all sensitive to allosteric inhibition by this set of compounds. One group of mutations (represented by D61G, S189A, L262R, and F285S) had no detectable effect on inhibitor potency (IC₅₀) for unactivated SHP2. A second group of mutations (represented by E76K, P491S, and S502P) resulted in a uniform reduction in inhibitor potency for all compounds in the set, although the most potent compounds retained double digit nanomolar activity against these mutants. For some SHP2 mutants there was a decrease in inhibitor potency in the presence of activating peptide relative to the corresponding apo form.

### Example 2

### Biochemical Sensitivity of SHP2 Mutants Predicts Cellular Sensitivity to Allosteric Inhibitor Compound B

### Methods

Generation of Isogenic SHP2 Expression Cell Lines

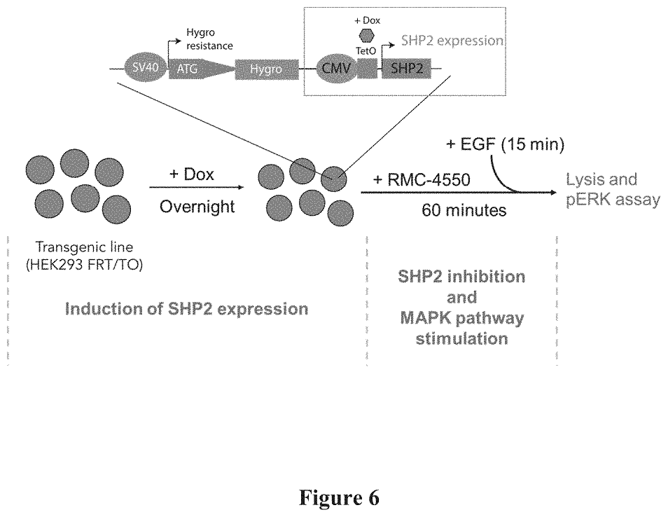

An experimental system was creating to test the activity of SHP2 mutants on an isogenic background (FIG. 6). The Flp-In T-REx-293 cell line was obtained from Gibco® and cultivated in high glucose DMEM™ containing 2 mM L-glutamine (Hyclone®), supplemented with 10% FBS (Hyclone®), 1% penicillin/streptomycin (Gibco®), 100 μg/mL Zeocin™ (Gibco®), and 15 μg/mL blasticidin (Gibco®) in a humidified cell culture incubator at 37° C., 5% CO2.

Wild type or mutant SHP2 variants were synthesized and subcloned into the pcDNA5/FRT/TO vector (ThermoFisher). Plasmids were co-transfected with the pOG44 Flp recombinase expression plasmid (ThermoFisher®) into Flp-In T-REx-293 cells using X-tremegene 9 DNA transfection reagent (Sigma®), according to the manufacturer's instructions. Cells that underwent successful recombination were selected in high glucose DMEM containing 2 mM L-glutamine, supplemented with 10% FBS and, 1% penicillin/streptomycin, 200 μg/mL hygromycin B (Gibco®), and 15 μg/mL blasticidin (Gibco®) (recombinant selection media) in a humidified cell culture incubator at 37° C., 5% CO2, until colonies were visually discernible. Colonies were expanded in recombinant selection media in a humidified cell culture incubator at 37° C., 5% CO2 to establish isogenic SHP2 variant expression cell lines (T-REx-293-SHP2).

Determination of Sensitivity to Compound B

One day prior to compound treatment, T-REx-293-SHP2 cells for each tested variant were harvested and seeded in high glucose DMEM containing 2 mM L-glutamine, supplemented with 0.1% FBS and, 1% penicillin/streptomycin, 200 μg/mL hygromycin B, and 15 μg/mL blasticidin in 96-well assay plates at a density of 25,000 cells/well. Expression of SHP2 constructs was induced by the addition of doxycycline (final concentration=0.1 μg/mL) (Sigma®) for 24 hours.

On the day of the experiment, cells were incubated in duplicate wells in the presence of increasing concentrations of Compound B (0.51 nM to 30 μM final assay concentration) or vehicle (final assay concentration 0.1% DMSO) at 37° C., 5% CO2 for 1 hour. For the final 5 minutes of drug treatment, cells were stimulated with 50 ng/mL Epidermal Growth Factor (Sigma®). After this incubation was complete, media was aspirated and cellular lysates prepared using lysis buffer provided with the AlphaLISA detection kit (PerkinElmer). ERK1/2 phosphorylation at Thr202/Tyr204 was assayed using the AlphaLISA SureFire Ultra HV pERK Assay Kit (Perkin Elmer®) following the manufacturer's instructions. Samples were read using an EnVision Multilabel Plate Reader (Perkin Elmer®) using standard AlphaLISA settings. Assay data was plotted and EC50 values were determined using four-parameter concentration-response model in GraphPad Prism 7. Data provided are mean+/−standard deviation of duplicate values from representative experiments.

Results

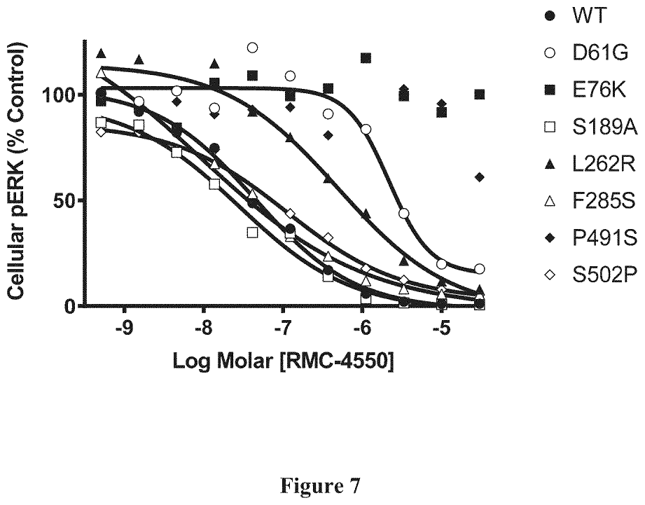

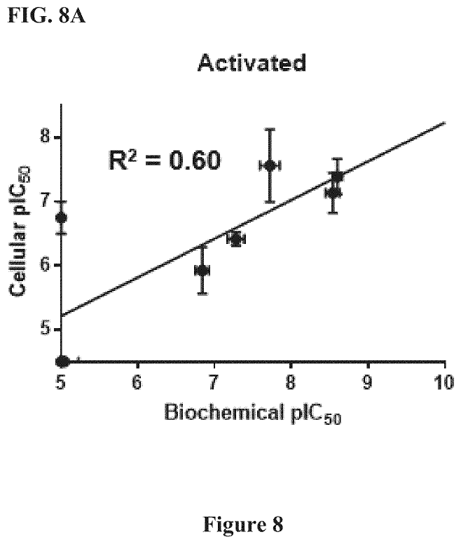

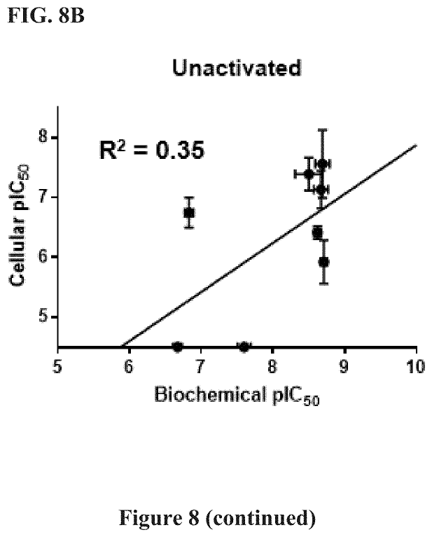

Fifteen stable, isogenic cell lines expressing different SHP2 variants were created using the FRT/TO system. Cells were incubated with Compound B prior to stimulation with EGF and measurement of cellular pERK levels by AlphaLISA (FIG. 7). Compound B potency for inhibition of mutants in cellular context correlated with biochemical potency for activated SHP2 variant (FIG. 8).

Overall, 8 of 13 cancer-associated mutants were sensitive to Compound B (IC₅₀<2 μM) (Table 9). Potency for inhibition of wild-type SHP2 in this system was comparable to endogenous SHP2 in other cell lines, and an engineered double mutant in the Compound B binding site (T253M/Q275L) was insensitive to inhibition.

TABLE 9
Sensitivity of SHP2 mutants to Compound B
Sensitivity of SHP2 mutants to Compound B

| | Biochemical | pERK IC50 | Bio- | |
|---|---|---|---|---|
| | IC50 (nM) | in HEK293 | chemical | Cellular |
| | 0.5 μM | Cells | sensi- | sensi- |
| Variant | NsCs*§ | (nM)*§ | tivity | tivity |
| WT | 2.88 | 49 | yes | yes |
| E76K | >10000 | >30000 | no | no |
| D61G | 145 | 1190 | yes | yes |
| S189A | 2.51 | 40.7 | yes | yes |
| L262R | 52.5 | 385 | yes | yes |
| P491S | 9120 | >30000 | no | no |
| F285S | 19.1 | 27.4 | yes | yes |
| S502P | >10000 | 179 | no | yes |
| A72V | ND | >30000 | ND | no |
| G60V | ND | 3700 | ND | no |
| E69K | ND | 713 | ND | yes |
| G503V | ND | >30000 | ND | no |
| T73I | ND | 626 | ND | yes |
| Q506P | ND | 228 | ND | yes |
| T253M/Q257L | >10000 | >30000 | no | no |

*Sensitive if IC50 < 2000 nM
§ND = Not determined

### CONCLUSIONS

A subset of clinically-relevant SHP2 mutants were sensitive to SHP2 allosteric inhibitors. Relatively more potent inhibitors of wild-type SHP2 were also more potent towards all mutants in this study. Sensitivity of SHP2 mutants to Compound B in cells correlated with biochemical sensitivity of activated enzyme. Results were consistent with a simple equilibrium model of SHP2 activation and inhibition driven by stability of an autoinhibited conformation

### EQUIVALENTS

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

1. A method of treating a subject having a disease or disorder associated with cells containing a mutant SHP2, comprising administering to the subject in need thereof an allosteric SHP2 inhibitor, wherein the mutant SHP2 comprises an allosteric inhibitor-sensitive mutation.

2. The method of claim 1, wherein the allosteric inhibitor-sensitive mutation is selected from the group consisting of F285S, L262R, S189A, D61G, E69K, T73I, Q506P, and a combination thereof.

3-5. (canceled)

6. The method of claim 1, wherein the cells are negative for an allosteric inhibitor-resistant mutation of SHP2.

7. The method of claim 6, wherein the allosteric inhibitor-resistant mutation is selected from the group consisting of E76K, P491S, S502P, and a combination thereof.

8-9. (canceled)

10. The method of claim 1, wherein the cells are determined to have the allosteric inhibitor-sensitive mutation prior to administering the SHP2 inhibitor.

11. The method of claim 1, wherein the cells are determined to not have the allosteric inhibitor-resistant mutation prior to administering the SHP2 inhibitor.

12. The method of claim 1, wherein the allosteric SHP2 inhibitor is selected from (i) Compound A; (ii) Compound B; (iii) Compound C; (iv) SHP099; (v) an allosteric SH1P2 inhibitor compound of any one of Formula I, of Formula II, of Formula III, of Formula I-VI, of Formula I-V2, of Formula I-W, of Formula I-X, of Formula I-Y, of Formula I-Z, of Formula IV, of Formula V, of Formula VI, of Formula IV-X, of Formula IV-Y, of Formula IV-Z, of Formula VII, of Formula VIII, of Formula IX, and of Formula X; (vi) TNO155; (vii) a compound from Table A, disclosed herein; (viii) a compound from Table A2, disclosed herein; and (ix) a combination thereof.

13. The method of claim 1, wherein the disease or disorder is selected from tumors of hemopoietic and lymphoid system; a myeloproliferative syndrome; a myelodysplastic syndromes; leukemia; acute myeloid leukemia; juvenile myelomonocytic leukemia; esophageal cancer; breast cancer; lung cancer; colon cancer; gastric cancer; neuroblastoma; bladder cancer; prostate cancer; glioblastoma; urothelial carcinoma; uterine carcinoma; adenoid and ovarian sereous cystadenocarcinoma; paraganglioma; phaeochromocytoma; pancreatic cancer; adrenocortical carcinoma; stomach adenocarcinoma; sarcoma; rhabdomyosarcoma; lymphoma; head and neck cancer; skin cancer; peritoneum cancer; intestinal cancer; thyroid cancer; endometrial cancer; cancer of the biliary tract; soft tissue cancer; ovarian cancer; central nervous system cancer; stomach cancer; pituitary cancer; genital tract cancer; urinary tract cancer; salivary gland cancer; cervical cancer; liver cancer; eye cancer; cancer of the adrenal gland; cancer of autonomic ganglia; cancer of the upper aerodigestive tract; bone cancer; testicular cancer; pleura cancer; kidney cancer; penis cancer; parathyroid cancer; cancer of the meninges; vulvar cancer and melanoma.

14. The method of claim 1, wherein the disease or disorder is an inherited developmental disorder selected from the group consisting of Noonan Syndrome and LEOPARD Syndrome.

15. The method of claim 1, wherein the allosteric SHP2 inhibitor is administered in an effective amount.

16. A method of identifying a subject with SHP2 mutations susceptible to a SHP2 inhibitor, comprising genotyping a biological sample from the subject for SHP2 mutations, wherein the subject is identified as susceptible to the SHP2 inhibitor if the SHP2 mutations comprise an allosteric inhibitor-sensitive mutation.

17. The method of claim 16, wherein the allosteric inhibitor-sensitive mutation is selected from the group consisting of F2S5S, L262R, S189A, D61G, E69K, T73I, Q506P, and a combination thereof.

18-20. (canceled)

21. The method of claim 16, wherein the method further comprises identifying the subject as not expressing a SHP2 allosteric inhibitor-resistant mutation.

22. The method of claim 21, wherein the SHP2 allosteric inhibitor-resistant mutation is selected from the group consisting of E76K, P491S, S502P, and a combination thereof.

23-24. (canceled)

25. The method of claim 16, wherein the allosteric SHP2 inhibitor is selected from (i) Compound A; (ii) Compound B; (iii) Compound C; (iv) SHP099; (v) an allosteric SHP2 inhibitor compound of any one of Formula I, of Formula II, of Formula III, of Formula I-V1, of Formula I-V2, of Formula I-W, of Formula I-X, of Formula I-Y, of Formula I-Z, of Formula IV, of Formula V, of Formula VI, of Formula IV-X, of Formula IV-Y, of Formula IV-Z, of Formula VII, of Formula VIII, of Formula IX, and of Formula X; (vi) TNO155; (vii) a compound from Table A1, disclosed herein; (viii) a compound from Table A2, disclosed herein; and (ix) a combination thereof.

26. A method of identifying a subject as resistant to an allosteric SHP2 inhibitor, comprising genotyping a biological sample from the subject for SHP2 mutations, wherein the subject is identified as resistant to the SHP2 inhibitor if the SHP2 mutations comprise an allosteric inhibitor-resistant mutation.

27. The method of claim 26, wherein the allosteric inhibitor-resistant mutation is selected from the group consisting of E76K, P491S, S502, and a combination thereof.

28-29. (canceled)

30. The method of claim 26, wherein the allosteric SHP2 inhibitor is selected from (i) Compound A; (ii) Compound B; (iii) Compound C; (iv) SHP099; (v) an allosteric SHP2 inhibitor compound of any one of Formula I, of Formula II, of Formula III, of Formula I-V1, of Formula I-V2, of Formula I-W, of Formula I-X, of Formula I-Y, of Formula I-Z, of Formula IV, of Formula V, of Formula VI, of Formula IV-X, of Formula IV-Y, of Formula IV-Z, of Formula VII, of Formula VIII, of Formula IX, and of Formula X; (vi) TNO155; (vii) a compound from Table A1, disclosed herein; (viii) a compound from Table A2, disclosed herein; and (ix) a combination thereof.

31-36. (canceled)

37. The method of claim 1, wherein the subject is identified as susceptible to an allosteric SHP2 inhibitor.

38. The method of claim 37, wherein the subject is identified by genotyping a biological sample from the subject for one or more SHP2 mutations and the one or more SHP2 mutations comprise an allosteric inhibitor-sensitive mutation.

39. The method of claim 37, wherein the subject is identified as not resistant to an allosteric SHP2 inhibitor.

40. The method of claim 39, wherein the subject is identified by genotyping a biological sample from the subject for one or more SHP2 mutations and the one or more SHP2 mutations comprise an allosteric inhibitor-resistant mutation.


SEQUENCE LISTING (3 sequences)

SEQ ID NO: 1 - protein, 593 aa, Homo sapiens [sequence omitted]

SEQ ID NO: 2 - protein, 9 aa, Artificial Sequence
  Synthesized activating peptide
LNYAQLWHA

SEQ ID NO: 3 - protein, 10 aa, Artificial Sequence
  Synthesized activating peptide
LTIYATIRRF